SEQUENCE LISTING (27 sequences)

SEQ ID NO: 1 - DNA, 1353 bp, Homo sapiens
  CDS (1)..(1353)
atgcagccgccaagggagaggctagtggtaacaggccgagctggatggatgggtatggggagaggggcaggacgttcagccctgggattctggccgaccctcgccttccttctctgcagcttccccgcagccacctccccgtgcaagatcctcaagtgcaactctgagttctggagcgccacgtcgggcagccacgccccagcctcagacgacacccccgagttctgtgcagccttgcgcagctacgccctgtgcacgcggcggacggcccgcacctgccggggtgacctggcctaccactcggccgtccatggcatagaggacctcatgagccagcacaactgctccaaggatggccccacctcgcagccacgcctgcgcacgctcccaccggccggagacagccaggagcgctcggacagccccgagatctgccattacgagaagagctttcacaagcactcggccacccccaactacacgcactgtggcctcttcggggacccacacctcaggactttcaccgaccgcttccagacctgcaaggtgcagggcgcctggccgctcatcgacaataattacctgaacgtgcaggccaccaacacgcctgtgctgcccggctcagcggccactgccaccagcaagctcaccatcatcttcaagaacttccaggagtgtgtggaccagaaggtgtaccaggctgagatggacgagctcccggccgccttcgtggatggctctaagaacggtggggacaagcacggggccaacagcctgaagatcactgagaaggtgtcaggccagcacgtggagatccaggccaagtacatcggcaccaccatcgtggtgcgccaggtgggccgctacctgacctttgccgtccgcatgccagaggaagtggtcaatgctgtggaggactgggacagccagggtctctacctctgcctgcggggctgccccctcaaccagcagatcgacttccaggccttccacaccaatgctgagggcaccggtgcccgcaggctggcagccgccagccctgcacccacagcccccgagaccttcccatacgagacagccgtggccaagtgcaaggagaagctgccggtggaggacctgtactaccaggcctgcgtcttcgacctcctcaccacgggcgacgtgaacttcacactggccgcctactacgcgttggaggatgtcaagatgctccactccaacaaagacaaactgcacctgtatgagaggactcgggacctgccaggcagggcggctgcggggctgcccctggccccccggcccctcctgggcgccctcgtcccgctcctggccctgctccctgtgttctgctag

SEQ ID NO: 2 - protein, 450 aa, Homo sapiens
MQPPRERLVVTGRAGWMGMGRGAGRSALGFWPTLAFLLCSFPAATSPCKILKCNSEFWSATSGSHAPASDDTPEFCAALRSYALCTRRTARTCRGDLAYHSAVHGIEDLMSQHNCSKDGPTSQPRLRTLPPAGDSQERSDSPEICHYEKSFHKHSATPNYTHCGLFGDPHLRTFTDRFQTCKVQGAWPLIDNNYLNVQATNTPVLPGSAATATSKLTIIFKNFQECVDQKVYQAEMDELPAAFVDGSKNGGDKHGANSLKITEKVSGQHVEIQAKYIGTTIVVRQVGRYLTFAVRMPEEVVNAVEDWDSQGLYLCLRGCPLNQQIDFQAFHTNAEGTGARRLAAASPAPTAPETFPYETAVAKCKEKLPVEDLYYQACVFDLLTTGDVNFTLAAYYALEDVKMLHSNKDKLHLYERTRDLPGRAAAGLPLAPRPLLGALVPLLALLPVFC

SEQ ID NO: 3 - DNA, 1437 bp, Homo sapiens
  CDS (1)..(1437)
atgataaggaagaagaggaagcgaagcgcgccccccggcccatgccgcagccacgggcccagacccgccacggcgcccgcgccgccgccctcgccggagcccacgagacctgcatggacgggcatgggcttgagagcagcaccttccagcgccgccgctgccgccgccgaggttgagcagcgccgccgccccgggctctgccccccgccgctggagctgctgctgctgctgctgttcagcctcgggctgctccacgcaggtgactgccaacagccagcccaatgtcgaatccagaaatgcaccacggacttcgtgtccctgacttctcacctgaactctgccgttgacggctttgactctgagttttgcaaggccttgcgtgcctatgctggctgcacccagcgaacttcaaaagcctgccgtggcaacctggtataccattctgccgtgttgggtatcagtgacctcatgagccagaggaattgttccaaggatggacccacatcctctaccaaccccgaagtgacccatgatccttgcaactatcacagccacgctggagccagggaacacaggagaggggaccagaaccctcccagttaccttttttgtggcttgtttggagatcctcacctcagaactttcaaggataacttccaaacatgcaaagtagaaggggcctggccactcatagataataattatctttcagttcaagtgacaaacgtacctgtggtccctggatccagtgctactgctacaaataagatcactattatcttcaaagcccaccatgagtgtacagatcagaaagtctaccaagctgtgacagatgacctgccggccgcctttgtggatggcaccaccagtggtggggacagcgatgccaagagcctgcgtatcgtggaaagggagagtggccactatgtggagatgcacgcccgctatatagggaccacagtgtttgtgcggcaggtgggtcgctacctgacccttgccatccgtatgcctgaagacctggccatgtcctacgaggagagccaggacctgcagctgtgcgtgaacggctgccccctgagtgaacgcatcgatgacgggcagggccaggtgtctgccatcctgggacacagcctgcctcgcacctccttggtgcaggcctggcctggctacacactggagactgccaacactcaatgccatgagaagatgccagtgaaggacatctatttccagtcctgtgtcttcgacctgctcaccactggtgatgccaactttactgccgcagcccacagtgccttggaggatgtggaggccctgcacccaaggaaggaacgctggcacattttccccagcagtggcaatgggactccccgtggaggcagtgatttgtctgtcagtctaggactcacctgcttgatccttatcgtgtttttgtag

SEQ ID NO: 4 - protein, 478 aa, Homo sapiens
MIRKKRKRSAPPGPCRSHGPRPATAPAPPPSPEPTRPAWTGMGLRAAPSSAAAAAAEVEQRRRPGLCPPPLELLLLLLFSLGLLHAGDCQQPAQCRIQKCTTDFVSLTSHLNSAVDGFDSEFCKALRAYAGCTQRTSKACRGNLVYHSAVLGISDLMSQRNCSKDGPTSSTNPEVTHDPCNYHSHAGAREHRRGDQNPPSYLFCGLFGDPHLRTFKDNFQTCKVEGAWPLIDNNYLSVQVTNVPVVPGSSATATNKITIIFKAHHECTDQKVYQAVTDDLPAAFVDGTTSGGDSDAKSLRIVERESGHYVEMHARYIGTTVFVRQVGRYLTLAIRMPEDLAMSYEESQDLQLCVNGCPLSERIDDGQGQVSAILGHSLPRTSLVQAWPGYTLETANTQCHEKMPVKDIYFQSCVFDLLTTGDANFTAAAHSALEDVEALHPRKERWHIFPSSGNGTPRGGSDLSVSLGLTCLILIVFL

SEQ ID NO: 5 - DNA, 1281 bp, Homo sapiens
  CDS (1)..(1281)
atgggggagccaggccagtcccctagtcccaggtcctcccatggcagtcccccaactctaagcactctcactctcctgctgctcctctgtggacatgctcattctcaatgcaagatcctccgctgcaatgctgagtacgtatcgtccactctgagccttagaggtgggggttcatcaggagcacttcgaggaggaggaggaggaggccggggtggaggggtgggctctggcggcctctgtcgagccctccgctcctatgcgctctgcactcggcgcaccgcccgcacctgccgcggggacctcgccttccattcggcggtacatggcatcgaagacctgatgatccagcacaactgctcccgccagggccctacagcccctcccccgccccggggccccgcccttccaggcgcgggctccggcctccctgccccggacccttgtgactatgaaggccggttttcccggctgcatggtcgtcccccggggttcttgcattgcgcttccttcggggacccccatgtgcgcagcttccaccatcactttcacacatgccgtgtccaaggagcttggcctctactggataatgacttcctctttgtccaagccaccagctcccccatggcgttgggggccaacgctaccgccacccggaagctcaccatcatatttaagaacatgcaggaatgcattgatcagaaggtgtatcaggctgaggtggataatcttcctgtagcctttgaagatggttctatcaatggaggtgaccgacctgggggatccagtttgtcgattcaaactgctaaccctgggaaccatgtggagatccaagctgcctacattggcacaactataatcattcggcagacagctgggcagctctccttctccatcaaggtagcagaggatgtggccatggccttctcagctgaacaggacctgcagctctgtgttggggggtgccctccaagtcagcgactctctcgatcagagcgcaatcgtcggggagctataaccattgatactgccagacggctgtgcaaggaagggcttccagtggaagatgcttacttccattcctgtgtctttgatgttttaatttctggtgatcccaactttaccgtggcagctcaggcagcactggaggatgcccgagccttcctgccagacttagagaagctgcatctcttcccctcagatgctggggttcctctttcctcagcaaccctcttagctccactcctttctgggctctttgttctgtggctttgcattcagtaa

SEQ ID NO: 6 - protein, 426 aa, Homo sapiens
MGEPGQSPSPRSSHGSPPTLSTLTLLLLLCGHAHSQCKILRCNAEYVSSTLSLRGGGSSGALRGGGGGGRGGGVGSGGLCRALRSYALCTRRTARTCRGDLAFHSAVHGIEDLMIQHNCSRQGPTAPPPPRGPALPGAGSGLPAPDPCDYEGRFSRLHGRPPGFLHCASFGDPHVRSFHHHFHTCRVQGAWPLLDNDFLFVQATSSPMALGANATATRKLTIIFKNMQECIDQKVYQAEVDNLPVAFEDGSINGGDRPGGSSLSIQTANPGNHVEIQAAYIGTTIIIRQTAGQLSFSIKVAEDVAMAFSAEQDLQLCVGGCPPSQRLSRSERNRRGAITIDTARRLCKEGLPVEDAYFHSCVFDVLISGDPNFTVAAQAALEDARAFLPDLEKLHLFPSDAGVPLSSATLLAPLLSGLFVLWLCIQ

SEQ ID NO: 7 - protein, 15 aa, Artificial
  Consensus sequence
XCXIXXCXXXXXSXT

SEQ ID NO: 8 - protein, 16 aa, Artificial
  Consensus 2
XCXALRXYAXCTXRTX

SEQ ID NO: 9 - DNA, 933 bp, Artificial
  hRGMA 47-90 Fc Plasmid
atggagacagacacactcctgctatgggtactgctgctctgggttccaggttccactggtgacgcggcccagccggccaggcgcgcgcgccgtacgaagcttccgtgcaagatcctcaagtgcaactctgagttctggagcgccacgtcgggcagccacgccccagcctcagacgacacccccgagttctgtgcagccttgcgcagctacgccctgtgcacgcggcggacggccttgaattctgcagatatcgagggacgaatggatccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 10 - protein, 310 aa, Artificial
  Synthetic Construct
METDTLLLWVLLLWVPGSTGDAAQPARRARRTKLPCKILKCNSEFWSATSGSHAPASDDTPEFCAALRSYALCTRRTALNSADIEGRMDPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 1167 bp, Artificial
  hRGMA 47-168 Fc Plasmid
atggagacagacacactcctgctatgggtactgctgctctgggttccaggttccactggtgacgcggcccagccggccaggcgcgcgcgccgtacgaagcttccgtgcaagatcctcaagtgcaactctgagttctggagcgccacgtcgggcagccacgccccagcctcagacgacacccccgagttctgtgcagccttgcgcagctacgccctgtgcacgcggcggacggcccgcacctgccggggtgacctggcctaccactcggccgtccatggcatagaggacctcatgagccagcacaactgctccaaggatggccccacctcgcagccacgcctgcgcacgctcccaccggccggagacagccaggagcgctcggacagccccgagatctgccattacgagaagagctttcacaagcactcggccacccccaactacacgcactgtggcctcttcggggacttgaattctgcagatatcgagggacgaatggatccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 12 - protein, 388 aa, Artificial
  Synthetic Construct
METDTLLLWVLLLWVPGSTGDAAQPARRARRTKLPCKILKCNSEFWSATSGSHAPASDDTPEFCAALRSYALCTRRTARTCRGDLAYHSAVHGIEDLMSQHNCSKDGPTSQPRLRTLPPAGDSQERSDSPEICHYEKSFHKHSATPNYTHCGLFGDLNSADIEGRMDPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 1197 bp, Artificial
  hRGMa 41-168 Fc Plasmid
atggagacagacacactcctgctatgggtactgctgctctgggttccaggttccactggtgacgcggcccagccggccaggcgcgcgcgccgtacgaagcttttccccgcagccacctccccgtgcaagatcctcaagtgcaactctgagttctggagcgccacgtcgggcagccacgccccagcctcagacgacacccccgagttctgtgcagccttgcgcagctacgccctgtgcacgcggcggacggcccgcacctgccggggtgacctggcctaccactcggccgtccatggcatagaggacctcatgagccagcacaactgctccaaggatggccccacctcgcagccacgcctgcgcacgctcccaccggccggagacagccaggagcgctcggacagccccgagatctgccattacgagaagagctttcacaagcactcggccacccccaactacacgcactgtggcctcttcggggacatcgaaggtcgtttgaattctgcagatatcgagggacgaatggatccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 14 - protein, 398 aa, Artificial
  Synthetic Construct
METDTLLLWVLLLWVPGSTGDAAQPARRARRTKLFPAATSPCKILKCNSEFWSATSGSHAPASDDTPEFCAALRSYALCTRRTARTCRGDLAYHSAVHGIEDLMSQHNCSKDGPTSQPRLRTLPPAGDSQERSDSPEICHYEKSFHKHSATPNYTHCGLFGDIEGRLNSADIEGRMDPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - DNA, 1014 bp, Artificial
  hRGMA 316-386 Fc Plasmid
atggagacagacacactcctgctatgggtactgctgctctgggttccaggttccactggtgacgcggcccagccggccaggcgcgcgcgccgtacgaagcttctgcggggctgccccctcaaccagcagatcgacttccaggccttccacaccaatgctgagggcaccggtgcccgcaggctggcagccgccagccctgcacccacagcccccgagaccttcccatacgagacagccgtggccaagtgcaaggagaagctgccggtggaggacctgtactaccaggcctgcgtcttcgacctcctcaccacgggcttgaattctgcagatatcgagggacgaatggatccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 16 - protein, 337 aa, Artificial
  Synthetic Construct
METDTLLLWVLLLWVPGSTGDAAQPARRARRTKLLRGCPLNQQIDFQAFHTNAEGTGARRLAAASPAPTAPETFPYETAVAKCKEKLPVEDLYYQACVFDLLTTGLNSADIEGRMDPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - DNA, 28 bp, Artificial
  PCR Primer
ggggaagcttttccccgcagccacctcc

SEQ ID NO: 18 - DNA, 45 bp, Artificial
  PCR Primer
gggggaattcaaacgaccttcgatgtccccgaagaggccacagtg

SEQ ID NO: 19 - DNA, 34 bp, Artificial
  PCR Primer
ggggaagcttccgtgcaagatcctcaagtgcaac

SEQ ID NO: 20 - DNA, 26 bp, Artificial
  PCR Primer
ccccgaattcaaggccgtccgccgcg

SEQ ID NO: 21 - DNA, 33 bp, Artificial
  PCR Primer
ccccgaattcaagtccccgaagaggccacagtg

SEQ ID NO: 22 - DNA, 26 bp, Artificial
  PCR Primer
ggggaagcttctgcggggctgccccc

SEQ ID NO: 23 - DNA, 31 bp, Artificial
  PCR Primer
ccccgaattcaagcccgtggtgaggaggtcg

SEQ ID NO: 24 - DNA, 20 bp, Artificial
  PCR Primer
atgcagccgccaagggagag

SEQ ID NO: 25 - DNA, 21 bp, Artificial
  PCR Primer
gcagaacacagggagcagggc

SEQ ID NO: 26 - protein, 9 aa, Artificial
  Consensus sequence
LXLCXXGCP

SEQ ID NO: 27 - protein, 28 aa, Artificial
  Consensus sequence
TAXXXCXEXXPVXDXYXXXCVFDXLXXG

(19) United States
(12) Patent Application Publication
(10) Pub. No.: US 2010/0322948 A1
(43) Pub. Date: Dec. 23, 2010

(54) Bone morphogenetic protein (BMP)-binding domains of proteins of the repulsive guidance molecule (RGM) protein family and functional fragments thereof, and use of same

(75) Inventors: Bernhard Mueller, Neustadt (DE); Gregor Schaffar, Mannheim (DE); Axel Meyer, Schwetzingen (DE); Martin Schmidt, Bensheim (DE)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: ABBOTT GMBH & CO. KG, Wiesbaden-Delkenhim, DE (DE)

(21) Appl. No.: 12/677,054

(22) PCT Filed: Sep. 8, 2008
(86) PCT No.: PCT/EP08/07339

§ 371 Date: Sep. 3, 2010

(30) Foreign Application Priority Data
Sep. 6, 2007 (EP) 07115856.2

(51) Int. Cl. A61K 39/395; C07K 14/47; C07K 5/08; C07K 5/10; C07K 7/06; C07K 7/08; C07K 19/00; C07K 16/18; A61K 38/02; A61P 3/10; A61P 25/02; A61P 19/08; A61P 7/06; A61P 9/00; A61P 17/06; A61P 1/04; A61P 17/14

(52) U.S. Cl. 424/172.1; 530/350; 530/331; 530/330; 530/329; 530/328; 530/327; 530/326; 530/325; 530/324; 530/387.3; 530/389.1; 514/16.7; 514/1.1; 514/13.5; 514/16.4; 514/18.6; 514/18.2; 514/6.9; 514/13.2; 514/20.7

(57) ABSTRACT

The present invention relates to the identification and use of bone morphogenetic protein (BMP)-binding domains of members of the repulsive guidance molecule (RGM) protein family, and polypeptide fragments and fusion proteins derived therefrom. The domains, i.e., peptide fragments and fusion proteins, according to the invention are suitable as agents for the active or passive immunization of individuals, or as diagnostic and therapeutic agents for use for diseases or medical conditions in whose origin or progression a member of the RGM family and a cellular receptor associated with this molecule, such as neogenin and/or BMP in particular, is involved. The invention further relates to monoclonal and polyclonal antibodies directed against the binding domains according to the invention, and against the polypeptides derived therefrom, and to methods for producing the polypeptides, fusion proteins, and antibodies according to the invention.

The present invention relates to the identification and use of bone morphogenetic protein (BMP)-binding domains of members of the repulsive guidance molecule (RGM) protein family, and polypeptide fragments and fusion proteins derived therefrom. The domains, i.e., peptide fragments and fusion proteins, according to the invention are suitable as agents for the active or passive immunization of individuals, or as diagnostic and therapeutic agents for use for diseases or medical conditions in whose origin or progression a member of the RGM family and a cellular receptor associated with this molecule, such as neogenin and/or BMP in particular, is involved. The invention further relates to monoclonal and polyclonal antibodies directed against the binding domains according to the invention, and against the polypeptides derived therefrom, and to methods for producing the polypeptides, fusion proteins, and antibodies according to the invention.

### BACKGROUND

The function of the members of the RGM protein family was first described by Monnier, P. P. et al, Nature, 419, 392-395, 2002. This family includes three previously known members, which are referred to as RGM A, RGM B (also called DRAGON), and RGM C (also called hemojuvelin) (Niederkofler V. et al., J. Neurosci. 24, 808-18, 2004). These are glycoproteins which are bound to the plasma membranes via a lipid anchor (glycosylphosphatidylinositol (GPI) anchor). The members of this protein family do not have an extensive sequence homology to other proteins, and structural features have been identified essentially in the following regions: an N-terminal signal peptide; an RGD sequence; a proteolytic cleavage site at the GDPH amino acid sequence; a structural homologue of the Willebrand factor domain (vWF D); a hydrophobic sequence in the vicinity of the C terminus; and a C-terminal GPI anchor consensus sequence (also see FIG. 2).

In humans, the coding sequences for RGM A on chromosome 15, for RGM B on chromosome 5, and for RGM C on chromosome 1 are localized. The following characteristic expression pattern is observed: RGM A and B are expressed in particular in the adult brain and spinal cord, RGM C is expressed in particular in the skeletal muscle, liver, and cardiac muscle, and RGM B is also expressed in the cartilaginous tissue.

RGM proteins were originally identified as candidate proteins, which play an important role in the formation of topographical neuronal projections (Stahl B. et al., Neuron 5: 735-43, 1990; Mueller B. K. et al., Curr. Biol. 6, 1497-1502, 1996; Mueller, B. K. in Molecular Basis of Axon Growth and Nerve Pattern Formation, Edited by H. Fujisawa, Japan Scientific Societies Press, 215-229, 1997). The ability of RGM proteins to act to repulse or inhibit growing nerve fibers was a crucial functional feature which played an important role in its isolation, cloning, and characterization. In simple cellular test systems the activity was easily demonstrated. RGM proteins had a repulsive or inhibitory effect in two different cellular assays. In the collapse test, RGM proteins were added to growing nerve fibers. The binding of RGM and the RGM receptor triggers a violent reaction in which all the membranous elements of the neuronal growth cone are retracted. The original expanded hand-like growth cone is transformed into a thin thread. In the presence of RGM the nerve fibers remain inhibited and retract strongly, and are no longer able to continue growth.

RGM proteins exert a portion of their effect by binding to the RGM receptor neogenin (Rajagopalan S. et al., Nat. Cell. Biol. 6, 756-62, 2004). Neogenin is closely related to the Deleted in Colorectal Cancer (DCC) receptor. Both receptors are members of the immunoglobulin superfamily, and have an extracellular, a transmembranal, and an intracellular domain. Both have been described as receptors for another ligand, netrin-1, but only neogenin, not DCC, binds RGM proteins. The extracellular domains of these receptors are composed of four immunoglobulin-like domains, followed by the six fibronectin repeat domains.

The function of RGM A is best understood in the nervous system, and its inhibitory effect on the growth of nerve fibers in very low concentrations is noteworthy. Injury to the central nervous system of growing humans and in adult rats results in an accumulation of RGM proteins at the lesion site (Schwab J. M. et al., Arch. Neurol., Vol. 62, 1561-1568, 2005; Schwab J. M. et al., Eur. J. Neurosci., 21, 387-98, 2005). New growth of the injured nerve fibers is thus prevented, resulting in permanent, more or less severe functional deficits, depending on the location of the lesion site. This activity of RGM which inhibits nerve fiber growth is mediated by binding to the receptor neogenin (Rajagopalan S. et al., loc. cit.). The same receptor mediates via the binding of netrin-1, but also mediates an opposite effect which stimulates the growth of nerve fiber. If the RGM A protein is neutralized by a polyclonal antibody at the lesion site in the spinal cord of rats, the nerve fibers regenerate over the injury site and form new synaptic contacts, resulting in significant functional improvements (Hata K. et al., J. Cell. Biol. 173, 47-58, 2006).

Recent findings indicate that the RGM proteins also play an important role in the central and peripheral nervous system, in the regulation of iron metabolism, in tumor diseases and inflammatory processes, and in the formation of bone and cartilaginous tissue.

The neurite growth-inhibiting domains of proteins of the RGM family are known from WO 2007/039256. Significant inhibitory activity was localized for RGM A, for example, in the sequence range 260-290.

It is also known that RGM A, B, and C are able to interact with various members of the BMP family. BMPs are members of the TGF-13 superfamily of ligands, which are involved in numerous physiological and pathophysiological processes. BMPs exercise their function via a specialized signal transduction path, which begins with the binding of the BMP ligand to a combination of two types of serin/threonin kinase receptors. An interaction with RGM has previously been demonstrated for BMP-2, -4, -5, -6, and -12 (see, for example, Babitt, J. L. et al., Nature Genetics, 2006, Vol. 48, 5, 531-539; Babitt, J. L. et al., J. Biol. Chem., 2005, Vol. 280, 33, 29820-29827; Babitt, J. L. et al., The Journal of Clinical Investigation, 2007, Vol. 117, 7, 1933-1939; Samad, T. A. et al., J. Biol. Chem., 2005, Vol. 280, 14, 14122-14129; and Halbrooks et al., J. Molecular Signaling 2, 4, 2007 (published in electronic form).

BMP-binding domains of RGM proteins have not been described heretofore.

The object of the present invention, therefore, is the localization of BMP-binding domains of the RGM proteins, and characterization thereof.

### BRIEF DESCRIPTION OF THE INVENTION

Surprisingly, the object stated above has been achieved by isolation and characterization of the BMP-binding domains of human RGM proteins, in particular RGM A, and of active polypeptide fragments thereof.

### DESCRIPTION OF THE FIGURES

FIG. 1 shows the sequence alignment of the human forms of RGM A (GenBank #NP_{—}064596.1), RGM B (GenBank #NP_{—}001012779), and RGM C (GenBank #NP_{—}998818.1).

FIG. 2 shows a schematic illustration of the structure of RGM molecules. Shown between the N-terminal signal peptide and the C-terminal GPI anchor are the RGD sequence, the von Willebrand factor domain (vWF D), and a hydrophobic sequence in the vicinity of the C terminus in front of the anchor regions. The neurite growth-inhibiting domain (OID) is located between vWFD and the hydrophobic region, in the range around positions 260-290. The corresponding amino acid positions for human RGM are shown below the diagram; the proteolytic cleavage site for human RGM A is located between amino acids 168 and 169.

FIG. 3 shows the results of an in vitro interaction assay between BMP 4 and various immobilized RGM A-Fc fusion proteins.

FIG. 4 shows the results of an in vitro interaction assay for comparison of the interaction of various immobilized RGM A-Fc fusion proteins with BMP-4 and BMP-2.

FIG. 5 shows the results of an in vitro interaction assay between immobilized BMP-4 and various RGM A-Fc fusion proteins.

FIG. 6 shows the results of an in vitro interaction assay between immobilized BMP-4 and different concentrations of fusion proteins 47-90-Fc (FIG. 6A), 47-168-Fc (FIG. 6B), and 316-386-Fc (FIG. 6C) according to the invention.

FIG. 7 shows the results of two different neurite growth tests using human neuroblastomacells (SH-SY5Y in FIG. 7A, NTera in FIG. 7B). Both hRGM A fragments, 786 (47-168) and 790 (316-386), inhibit the neurite growth, with fragment 47-168-Fc having a much stronger effect.

FIG. 8A shows the results of immunoblotting experiments with monoclonal antibodies 4A9. MAB 4A9 recognizes fragment 47-168 (lane 5) of human RGM A. 4A9 also binds to additional hRGM A fragments, 70-120 (lane 2) and fragment 47-90 (lane 4) and recognizes full length hRGM A (47-422) (lanes 6 and 9). Molecular weight standards are indicated in lane 1. FIG. 8B shows the results of ELISA experiments, wherein the interaction of full length h RGM A and hBMP-4 is completed inhibited in a dose dependent manner by MAB 4A9.

FIG. 9 shows the dose-dependent response of luciferase activity resulted after treatment of C3H-B12 with rhBMP-2 at different concentrations (from 0 to 50 ng/ml).

FIG. 10 shows the antagonistic effect of peptide fragments of hRGM A on BMP signaling using BRE-Luc assay as determined by exposing C3H-B12 to different concentrations of test compounds for 24 hours and monitoring changes in the respective cell luciferase activity.

### DETAILED DESCRIPTION OF THE INVENTION

I. Explanation of General Terms

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

Generally, nomenclatures used in connection with, and techniques of, cell and tissue culture, molecular biology, immunology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

That the present invention may be more readily understood, selected terms are defined below.

The term “polypeptide” as used herein, refers to any polymeric chain of amino acids. The terms “peptide” and “protein” are used interchangeably with the term polypeptide and also refer to a polymeric chain of amino acids. The term “polypeptide” encompasses native or artificial proteins, protein fragments and polypeptide analogs of a protein sequence. A polypeptide may be monomeric or polymeric.

The term “isolated protein” or “isolated polypeptide” is a protein or polypeptide that by virtue of its origin or source of derivation is not associated with naturally associated components that accompany it in its native state; is substantially free of other proteins from the same species; is expressed by a cell from a different species; or does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

The term “recovering” as used herein, refers to the process of rendering a chemical species such as a polypeptide substantially free of naturally associated components by isolation, e.g., using protein purification techniques well known in the art.

Within the scope of the present invention, the term “receptors” refers in particular to surface molecules bound to a cell membrane which are able to interact with a ligand, which is soluble, for example, and which as the result of this interaction are able to trigger a signal directed into the interior of the cell, for example, or a signal cascade (also referred to as “signaling”).

“Ligand” refers to a natural, i.e., produced in vivo, or synthesized, low- or high-molecular binding partner for a “receptor.” The ligand is preferably freely mobile in the extracellular environment.

“Immunogen” refers to a peptide fragment according to the invention in glycosylated or non-glycosylated form which is suitable for inducing the formation of antibodies against the immunogen. Binding of the immunogen (in the form of a hapten) to a macromolecular substrate may be advantageous.

“Epitope” or antigen determinant refers to the region of an antigen, such as a protein, for example, which determines the specificity of an antibody. If this epitope is newly formed in a segment of the protein or expressed on the accessible molecular surface, for example as the result of external influences such as an interaction of a protein with a ligand, this is referred to as a “neoepitope.” in particular, the term “epitope” or “antigenic determinant” includes any polypeptide determinant capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three-dimensional structural characteristics, and/or specific charge characteristics. An epitope is a region of an antigen that is bound by an antibody. In certain embodiments, an antibody is said to specifically bind an antigen when it preferentially recognizes its target antigen in a complex mixture of proteins and/or macromolecules.

As used herein, the term “neutralizing” refers to neutralization of biological activity of a target protein when a binding protein specifically binds the target protein. Preferably a neutralizing binding protein is a neutralizing antibody whose binding to RGM A, B or C molecule results in inhibition of a biological activity of said RGM molecule. Preferably the neutralizing binding protein binds RGM and reduces a biologically activity of RGM by at least about 20%, 40%, 60%, 80%, 85% or more. Inhibition of a biological activity of RGM by a neutralizing binding protein can be assessed by measuring one or more indicators of RGM biological activity well known in the art.

The term “activity” includes activities such as the binding specificity/affinity of an antibody for an antigen, for example, an anti-RGM antibody that binds to an RGM antigen and/or the neutralizing potency of an antibody, for example, an anti-RGM A antibody whose binding to RGM A inhibits the biological activity of RGM A.

“Domain” of a protein or antibody refers to a complex structure, delimited within the protein, which is formed by structural elements, such as for example alpha helix and/or beta sheet elements.

Unless indicated otherwise, the term “RGM protein according to the invention” encompasses the BMP-binding domains as well as polypeptides, derived therefrom, of a member of the family of RGM molecules, in particular RGM A, B, and C. In particular, functional polypeptide fragments which “stimulate” a BMP signal transduction path are included. The polypeptides according to the invention may also encompass “inhibitively active” polypeptides, in particular those which bind to neogenin, or which have activity which inhibits nerve fiber growth (elucidated by means of a neurite growth test, described herein).

“Binding” of the domains or polypeptides according to the invention is understood in the broadest sense as an interaction, optionally limited as a function of time, of any type with a binding partner such as BMP or neogenin, for example. The binding may be specific or nonspecific, preferably specific. Such binding is detected using suitable binding assays, such as the binding tests described in the experimental section herein. In particular, the domains or polypeptides according to the invention may be brought into contact with the particular binding partner by forming covalent or noncovalent interactions, such as ionic and/or hydrophobic interactions, for example. In particular, the interaction may be sufficient to modulate, i.e., influence positively or negatively, so as to promote or partially or completely inhibit a characteristic mediated by the binding partner, such as a biological function, for example an interaction of the binding partner with a third partner.

Binding according to the invention is “specific” in particular when the quantity of different binding partners or different binding partner classes is numerically limited. In particular, the binding should not be carried out with more than 10, such as, for example, 1, 2, 3, 4, or 5 different binding partners or binding partner classes. For example, BMP represents a binding partner class. Specificity is likewise present when, although an interaction takes place with multiple binding partners, the interaction has sufficient intensity to influence a biological function in the above sense only with a limited number of binding partners. For example, specificity is present when a domain or a polypeptide according to the invention containing at least one BMP, in particular selected from BMP-2, BMP-4, BMP-5, BMP-6, and BMP-12, binds in particular to BMP-2 and/or BMP-4; and/or binds to neogenin.

“Inhibiting” or “inhibitively active” polypeptides are those which reduce or completely inhibit the growth of nerve cells in a nerve cell growth test, described herein.

The above-described “stimulating” and “inhibitory” activity may be specified independently from one another for a given polypeptide; however, it is preferred that the BMP signal path-“stimulating” activity is always present, and the nerve cell growth-“inhibiting” activity is only optionally present.

“BMP signal transduction”-stimulating activity is an activity which may be triggered by at least one BMP protein selected from BMP-2, -4, -5, -6, and -12. This stimulating activity is present when a BMP binding polypeptide according to the invention, elucidated by an in vitro binding test described herein, interacts with at least one BMP molecule selected from BMP-2, -4, -5, -6, and -12.

Unless indicated otherwise, “RGM” stands for RGM A, B, and C, in particular RGM A.

“Neogenin” and “neogenin receptor” are synonymous terms, and refer in particular to mammalian neogenin, in particular human neogenin.

A “functional linkage” of a BMP-binding domain or a BMP-binding polypeptide with another amino acid sequence is understood in particular as a covalent, for example, peptidic linkage which permits binding of the domain or the polypeptide to at least one BMP molecule selected from BMP-2, -4, -5, -6, and -12 and/or Neogenin.

The term “regulate” and “modulate” are used interchangeably, and, as used herein, refers to a change or an alteration in the activity of a molecule of interest (e.g., the biological activity of RGM A). Modulation may be an increase or a decrease in the magnitude of a certain activity or function of the molecule of interest. Exemplary activities and functions of a molecule include, but are not limited to, binding characteristics, enzymatic activity, cell receptor activation, and signal transduction.

Correspondingly, the term “modulator,” as used herein, is a compound capable of changing or altering an activity or function of a molecule of interest (e.g., the biological activity of RGM A). For example, a modulator may cause an increase or decrease in the magnitude of a certain activity or function of a molecule compared to the magnitude of the activity or function observed in the absence of the modulator. In certain embodiments, a modulator is an inhibitor, which decreases the magnitude of at least one activity or function of a molecule. Exemplary inhibitors include, but are not limited to, proteins, peptides, antibodies, peptibodies, carbohydrates or small organic molecules. Peptibodies are described, e.g., in WO01/83525.

The term “agonist”, as used herein, refers to a modulator that, when contacted with a molecule of interest, causes an increase in the magnitude of a certain activity or function of the molecule compared to the magnitude of the activity or function observed in the absence of the agonist. Particular agonists of interest may include, but are not limited to, RGM A polypeptides or polypeptides, nucleic acids, carbohydrates, or any other molecules that bind to RGM A

The term “antagonist” or “inhibitor”, as used herein, refer to a modulator that, when contacted with a molecule of interest causes a decrease in the magnitude of a certain activity or function of the molecule compared to the magnitude of the activity or function observed in the absence of the antagonist. Particular antagonists of interest include those that block or modulate the biological or immunological activity of RGM A. Antagonists and inhibitors of RGM A may include, but are not limited to, proteins, nucleic acids, carbohydrates, or any other molecules, which bind to RGM A.

As used herein, the term “effective amount” refers to the amount of a therapy which is sufficient to reduce or ameliorate the severity and/or duration of a disorder or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g., prophylactic or therapeutic agent).

The term “sample”, as used herein, is used in its broadest sense. A “biological sample”, as used herein, includes, but is not limited to, any quantity of a substance from a living thing or formerly living thing. Such living things include, but are not limited to, humans, mice, rats, monkeys, dogs, rabbits and other animals. Such substances include, but are not limited to, blood, serum, urine, synovial fluid, cells, organs, tissues, bone marrow, lymph nodes and spleen.

II. Specialized Subject Matter of the Invention

A first subject matter of the invention concerns bone morphogenetic protein (BMP)-binding domains or binding peptide fragments of the repulsive guidance molecule (RGM) in glycosylated or in particular non-glycosylated form, preferably derived from mammal RGM, for example humans, rats or mice, or poultry, for example chickens. Unless indicated otherwise, the term “binding domain” encompasses any polypeptides, binding to at least one BMP, that are derived from an RGM.

One preferred embodiment relates to BMP-binding domains derived from human RGM A according to SEQ ID NO: 2, human RGM B according to SEQ ID NO: 4, or human RGM C according to SEQ ID NO: 6. The binding domains encompass in particular an amino acid sequence having a length of up to 170, for example up to 125, up to 100, up to 80, up to 60, up to 50, up to 40, up to 30, up to 20 or up to 10 preferably contiguous amino acid radicals from an amino acid sequence range of RGM, specifically, N-terminal with respect to the vWF domain or C-terminal with respect to the nerve fiber growth-inhibiting domain (OID) of the RGM, such as RGM A in particular, or from the corresponding sequence ranges of RGM B and C which may be derived by a sequence alignment.

The subject matter of the invention in particular concerns BMP-binding domains having a length of approximately 30 to 150 contiguous amino acid radicals, as well as functional derivatives thereof and fusion proteins containing at least one BMP-binding domain in functional linkage with at least one additional amino acid sequence which is different.

The subject matter of the invention also concerns BMP-binding domains which are characterized by at least one of the following partial sequences according to SEQ ID NO: 7 and 8:

(SEQ ID NO: 7)
X₁C(K/R)IX₂(K/R)CX₃(S/T/A)(E/D)(F/Y)X₄SX₅T

where X₁ through X₅ stand for any given amino acid radicals; or

| | |
|---|---|
| X₆CX₇ALRX₈YAX₉CTX₁₀RTX₁₁ | (SEQ ID NO: 8) |

where X₆ through X₁₁ stand for any given amino acid radicals;

or a partial sequence of formula

(SEQ ID NO: 7)-Link1-(SEQ ID NO: 8)

where Link1 stands for a SEQ ID NO: 7- and 8-bridging amino acid sequence containing 10 to 45, for example 13 to 28, any given contiguous amino acid radicals.

In particular,

X₁ stands for Pro or Gln

X₂ stands for Leu or Gln

X₃ stands for Asn or Thr

X₄ stands for Val or Trp

X₅ stands for Ser, Ala, or Leu

X₆ stands for Phe or Leu

X₇ stands for Ala, Lys, or Arg

X₈ stands for Ser or Ala

X₉ stands for Leu or Gly

X₁₀ stands for Arg or Gln, and/or

X₁₁ stands for Ala or Ser.

The following specific examples of SEQ IQ NO: 7 are listed:

PCKILKCNSEFWSAT
QCRIQKCTTDFVSLT
QCKILRCNAEYVSST

The following specific examples of SEQ IQ NO: 8 are listed:

FCAALRSYALCTRRTA
FCKALRAYAGCTQRTS
LCRALRSYALCTRRTA

The following specific examples of the Link1 linker are listed:

SGSHAPASDDTPE
SHLNSAVDGFDSE
LSLRGGGSSGALRGGGGGGRGGGVGSGG

Examples of BMP-binding domains include an amino acid sequence in the range of amino acid positions 30 to 180 according to SEQ ID NO: 2, in the range of amino acid positions 80 to 230 according to SEQ ID NO: 4, or in the range of amino acid positions 20 to 150 according to SEQ ID NO: 6, or functional neogenin receptor-binding fragments thereof. These binding domains (and fragments derived therefrom) are also referred to as high-affinity BMP-binding domains. In particular, the high-affinity BMP-binding domains may also have a high-affinity interaction with neogenin, and may therefore also be referred to as high-affinity neogenin-binding domains. On the other hand, one example of a low-affinity neogenin-binding domain is the RGM A fragment 218-284 according to SEQ ID NO: 2, described in WO 2007/039256, the disclosure of which is expressly referenced herein. Without being limited thereto, low-affinity binding is present, for example, when K_{D} (dissociation constant)>1 μM, such as 2 to 10 μM, for example; high-affinity binding may be present, for example, when K_{D}<10 nM, such as 1 to 9 nM, for example.

Examples of high-affinity BMP-binding domains are those which contain one of the following amino acid sequences of SEQ ID NO: 2:

Amino acid position from approximately 47 to approximately 168, or approximately 41 to approximately 168

Amino acid position from approximately 47 to approximately 90, or approximately 41 to approximately 90 or

Amino acid position from approximately 75 to approximately 121;

or one of the following amino acid sequences of SEQ ID NO: 4:

Amino acid position from approximately 94 to approximately 209

Amino acid position from approximately 94 to approximately 137 or

Amino acid position from approximately 122 to approximately 168;

one of the following amino acid sequences of SEQ ID NO: 6:

Amino acid position from approximately 36 to approximately 172

Amino acid position from approximately 36 to approximately 94 or

Amino acid position from approximately 80 to approximately 125;

or a functional BMP binding fragment thereof, for example a fragment of one of the above sequences, for which the C and/or N terminus may be shortened by 1, 2, 3, 4, 5, 6, 7, 8, 9, 10, 12, 15, or up to 20 amino acid radicals without losing the capability for BMP binding (detectable in a binding test described herein).

Other specific examples of domains according to the invention are BMP-binding domains or binding fragments thereof containing at least 10 contiguous amino acid radicals, as for example 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 40, 41, 2,43, 44, or 45 contiguous radicals, from the sequence range from approximately position 47 to approximately 168 according to SEQ ID NO: 2, from the sequence range from approximately position 94 to approximately 209 according to SEQ ID NO: 4, or from the sequence range from approximately position 36 to 172 according to SEQ ID NO: 6.

This is further exemplified in the Table A attached at the end of the specification by making reference to SEQ ID NO: 2.

As non-limiting number of examples of suitable fragments in the range of amino acid positions 60 to 120 are are mentioned below (the first and the last amino acid residue are given):

60-120, 61-120, 62-120, 64-120, 65-120, 66-120, 67-120, 68-120, 69-120, 70-120;

60-119, 61-119, 62-119, 64-119, 65-119, 66-119, 67-119, 68-119, 69-119, 70-119;

60-118, 61-118, 62-118, 64-118, 65-118, 66-118, 67-118, 68-118, 69-118, 70-118;

60-117, 61-117, 62-117, 64-117, 65-117, 66-117, 67-117, 68-117, 69-117, 70-117;

60-116, 61-116, 62-116, 64-116, 65-116, 66-116, 67-116, 68-116, 69-116, 70-116;

60-115, 61-115, 62-115, 64-115, 65-115, 66-115, 67-115, 68-115, 69-115, 70-115;

60-114, 61-114, 62-114, 64-114, 65-114, 66-114, 67-114, 68-114, 69-114, 70-114;

60-90, 61-90, 62-90, 64-90, 65-90, 66-90, 67-90, 68-90, 69-90, 70-90;

60-89, 61-89, 62-89, 64-89, 65-89, 66-89, 67-89, 68-89, 69-89, 70-89;

60-88, 61-88, 62-88, 64-88, 65-88, 66-88, 67-88, 68-88, 69-88, 70-88;

60-87, 61-87, 62-87, 64-87, 65-87, 66-87, 67-87, 68-87, 69-87, 70-87;

60-86, 61-86, 62-86, 64-86, 65-86, 66-86, 67-86, 68-86, 69-86, 70-119;

60-85, 61-85, 62-85, 64-85, 65-85, 66-85, 67-85, 68-85, 69-85, 70-85;

60-84, 61-84, 62-84, 64-84, 65-84, 66-84, 67-84, 68-84, 69-84, 70-84;

60-83, 61-83, 62-83, 64-83, 65-83, 66-83, 67-83, 68-83, 69-83, 70-83;

60-82, 61-82, 62-82, 64-82, 65-82, 66-82, 67-82, 68-82, 69-82, 70-82;

60-81, 61-81, 62-81, 64-81, 65-81, 66-81, 67-81, 68-81, 69-81, 70-81;

60-80, 61-80, 62-80, 64-80, 65-80, 66-80, 67-80, 68-80, 69-80, 70-80;

60-79, 61-79, 62-79, 64-79, 65-79, 66-79, 67-79, 68-79, 69-79, 70-79;

60-78, 61-78, 62-78, 64-78, 65-78, 66-78, 67-78, 68-78, 69-78;

60-77, 61-77, 62-77, 64-77, 65-77, 66-77, 67-77, 68-77;

60-76, 61-76, 62-76, 64-76, 65-76, 66-76, 67-76;

60-75, 61-75, 62-75, 64-75, 65-75, 66-75;

60-74, 61-74, 62-74, 64-74, 65-74;

60-73, 61-73, 62-73, 64-73;

60-72, 61-72, 62-72;

60-71, 61-71;

60-70;

60-69.

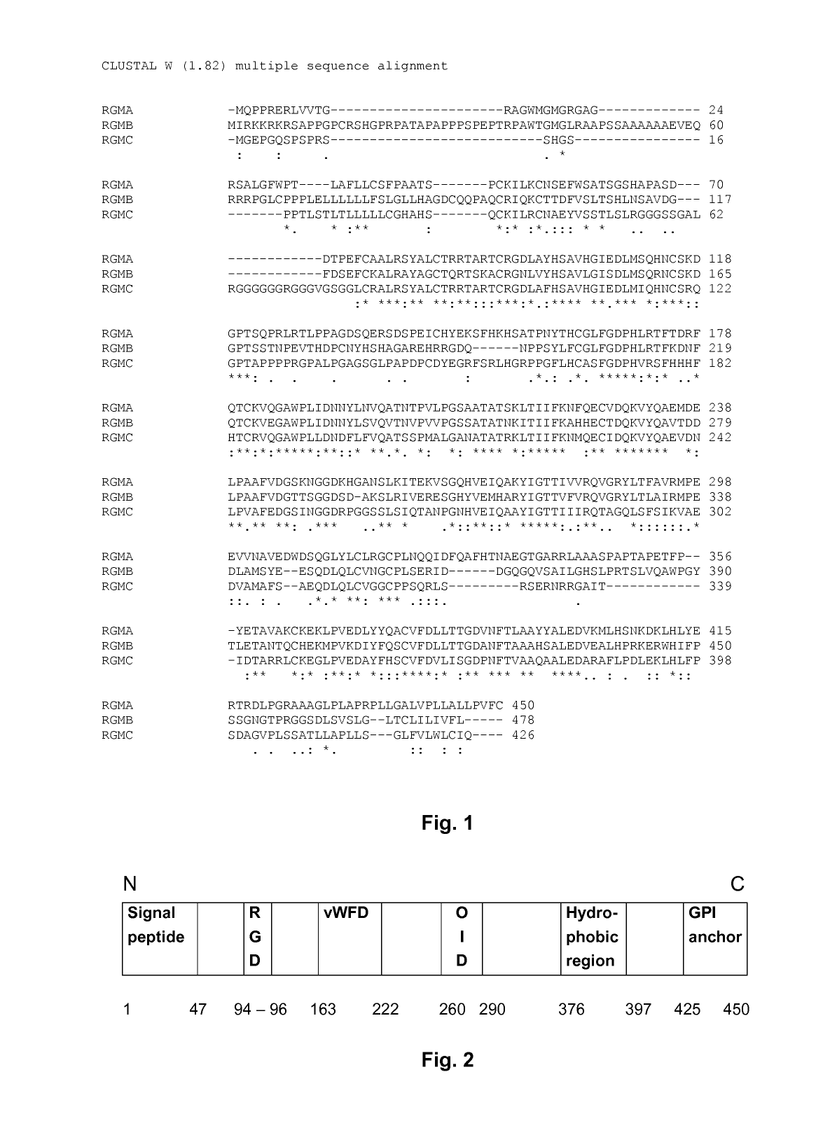

Similar fragments are analoguoulsly derivable from corresponding portions of SEQ ID NO: 4 and SEQ ID NO: 6, based on the sequence alignment of FIG. 1.

A further subject matter of the invention concerns BMP-binding domains situated, for example, C terminal with respect to the OID (see FIG. 2). These binding domains (and fragments derived therefrom) are also referred to as low-affinity BMP-binding domains, since their binding affinity relative to the above-referenced high affinity BMP-binding domains may be lower under comparable test conditions.

The subject matter of the invention, therefore, also concerns BMP-binding domains which are characterized by at least one of the following partial sequences according to SEQ ID NO: 26 and 27:

| | |
|---|---|
| LX₂₀LC(V/L)X₂₁GCP | (SEQ ID NO: 26) |

where X₂₀ and X₂₁ stand for any given amino acid radicals; or

(SEQ ID NO: 27)
TAX₂₂X₂₃X₂₄C(K/H)EX₂₅(L/M)PV(E/K)DX₂₆Y(F/Y)(Q/H)(A/S)
CVFD(V/L)LX₂₇(T/S)G

where X₂₂ to X₂₇ stand for any given amino acid radicals;

or a partial sequence of formula

(SEQ ID NO: 26)-Link2-(SEQ ID NO: 27)

where Link2 stands for a SEQ ID NO: 26 and 27-bridging amino acid sequence containing 10 to 45, for example approximately 19 to 38, any given contiguous amino acid radicals.

In particular,

X₂₀ stands for Tyr or Gln

X₂₁ stands for Arg, Asn or Gly

X₂₂ stands for Arg, Asn or Val

X₂₃ stands for Ala, Thr or Arg

X₂₄ stands for Lys, Gln or Leu

X₂₅ stands for Lys or Gly

X₂₆ stands for Leu, Ile or Ala and/or

X₂₇ stands for Thr or Ile

The following specific examples of SEQ IQ NO: 26 are listed:

LYLCLRGCP
LQLCVNGCP
LQLCVGGCP

The following specific examples of SEQ IQ NO: 27 are listed:

TAVAKCKEKLPVEDLYYQACVFDLLTTG
TANTQCHEKMPVKDIYFQSCVFDLLTTG
TARRLCKEGLPVEDAYFHSCVFDVLISG

The following specific examples of the Link2 linker are listed:

LNQQIDFQAFHTNAEGTGARRLAAASPAPTAPETFPYE
LSERIDDGQGQVSAILGHSLPRTSLVQAWPGYTLE
PSQRLSRSERNRRGAITID

Examples of low-affinity domains according to the invention are BMP-binding domains or binding fragments thereof containing at least 10 contiguous amino acid radicals from the sequence range approximately from position 316 to approximately 386 according to SEQ ID NO: 2, from the sequence range approximately from position 350 to approximately 421 according to SEQ ID NO: 4, or from the sequence range approximately from position 314 to 369 according to SEQ ID NO: 6.

Examples of the above-referenced “binding fragments containing at least 10 amino acid radicals” are those containing, for example, 10-50, 10-40, 10-30, 10-25, 10-20, or 10-15, in particular 10, 11, 12, 13, 14, or 15 contiguous amino acid radicals from one of the above-referenced peptides or sequence ranges.

The subject matter of the invention in particular concerns BMP-binding domains which bind to at least one BMP selected from BMP-2, BMP-4, BMP-5, BMP-6, and BMP-12, in particular BMP-2 and/or BMP-4; and BMP-binding domains which also bind to neogenin.

A further subject matter of the invention concerns antigenic polypeptide fragments of the BMP-binding domains according to the above definition. In particular, antigenic polypeptide fragments which may be used for producing immunoglobulin molecules and which modulate the binding of RGM to a BMP receptor molecule, in particular which partially or completely agonize or antagonize and optionally modulate the binding to the neogenin receptor, in particular which partially or completely antagonize same, represent subject matter of the invention. Named as examples are antigenic polypeptide fragments containing at least 10, for example 10-30, 10-25, 10-20, or 10-15, as for example 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 40, 41, 42, 43, 44, or 45 contiguous amino acid radicals of one of the above-referenced peptides or peptides stated in the annexed Table A derived from SEQ ID NO:2, or similarily derived from SEQ ID NO: 4 or 6.

A further subject matter of the invention concerns the use of a BMP-binding domain according to the above definition, or the use of a polypeptide fragment according to the above definition, for producing a polyclonal antiserum or a monoclonal antibody against RGM, wherein the antiserum or the antibody in particular modulates, preferably partially or completely antagonizes, the binding of RGM to the neogenin receptor.

The subject matter of the invention also concerns polyclonal antisera or monoclonal antibodies against RGM according to the above definition for diagnostic or therapeutic use.

A further subject matter of the invention concerns fusion proteins containing at least one first biologically active polypeptide, selected from BMP-binding domains according to the above definition, which is operatively linked (i.e., at the N or C terminus) to a second polypeptide, selected from a mono- or polyvalent carrier polypeptide or a second biologically active polypeptide. The polyvalent carrier in particular contains at least one Fc or Fc” molecule of an immunoglobulin, in particular from a mammal, such as derived in particular from a human immunoglobulin, wherein each of the two polypeptide chains thereof is operatively linked to the same or different BMP-binding domains according to the above definition.

A further subject matter of the invention concerns the use of a BMP-binding domain according to the above definition, or the use of a polypeptide fragment according to the above definition for producing a polyclonal antiserum or monoclonal antibody against RGM, wherein the antiserum or the antibody modulates the binding of RGM to neogenin (i.e., the neogenin receptor).

The subject matter of the invention also concerns a polyclonal antiserum or a monoclonal antibody against RGM according to the above definition for diagnostic or therapeutic use.

The subject matter of the invention also concerns the use of a polyclonal antiserum or a monoclonal antibody according to the above definition for producing a pharmaceutical agent for the diagnosis or therapy of diseases or disease stages which are mediated by an interaction of the neogenin receptor (neogenin) with RGM or an RGM fragment, in particular diseases or disease stages selected from
a) Mechanical injuries to the skull, brain, and spinal cord,
b) Chronic diseases selected from neurodegenerative, inflammatory, or autoimmune diseases,
c) Disorders of neuronal regeneration, axonal sprouting, neurite extension, and neuronal plasticity,
d) Tumor diseases and tumor metastasis.

The subject matter of the invention further concerns the use of a BMP-binding domain according to the above definition or a fusion protein according to the above definition for producing an agent for the diagnosis or therapy of diseases or disease stages which are mediated by a faulty or impaired interaction of RGM or an RGM fragment with the associated receptor (neogenin or BMP), when the diseases or disease stages are selected in particular from the following:
a) Altered neuritogenesis processes in psychotic conditions and chronic pain states caused by excessive neurite sprouting and/or pathological synaptogenesis;
b) Diseases associated with faulty iron metabolism;
c) Diseases associated with impaired bone growth;
d) Diseases associated with degenerative changes in cartilage;
e) Diseases associated with damage to the intervertebral disks and vertebral bodies;
f) Diseases associated with deregulated, uncontrolled cell migration processes.

A further subject matter of the invention concerns the use of a BMP-binding domain or a binding fragment thereof according to the above definition, or a fusion protein according to the above definition, for producing an agent for the diagnosis or therapy of diseases or disease stages which may be treated by stimulation or amplification of the BMP signal path (by binding to BMP-2, BMP-4, BMP-5, BMP-6, and/or BMP[-12], in particular to BMP-2 and/or 4), in particular for the treatment of diseases involving impaired bone growth, or for the treatment of bone fractures.

A further subject matter of the invention concerns the use of a BMP-binding domain or a binding fragment thereof according to the above definition, or a fusion protein according to the above definition, for producing an agent for the diagnosis or therapy of autoimmune diseases, in particular those selected from spondylitis ankylosans, antiphospholipid syndrome, Addison's disease, autoimmune hemolytic anemia, autoimmune hepatitis, autoimmune lymphoproliferative syndrome (ALPS), autoimmune thrombocytopenic purpura (ATP), Behçet's disease, bullous pemphigoid, cardiomyopathy, celiac disease, dermatitis herpetiformis, chronic fatigue immune dysfunction syndrome (CFIDS), chronic inflammatory demyelinating polyneuropathy (CIDP), cicatricial pemphigoid, systemic sclerosis (CREST syndrome), cold agglutination disease, Crohn's disease, cutaneous vasculitis, Degos disease, dermatomyositis, juvenile dermatomyositis, lupus erythematosus discoides, essential mixed cryoglobulinemia, fibromyalgia, Goodpasture's syndrome, Graves' disease, Guillain-Barré syndrome, Hashimoto's thyroiditis, idiopathic pulmonary fibrosis, idiopathic thrombocytopenia purpura (ITP), immunoglobulin A nephropathy, insulin-dependent diabetes mellitus, juvenile arthritis, Kawasaki disease, lichen planus, membranous glomerulonephritis, Meniere's disease, mixed connective tissue disease, multifocal motor neuropathy, multiple sclerosis, myasthenia gravis, pemphigus vulgaris, pernicious anemia, polyarteritis nodosa, polychondritis, polyglandular syndrome, polymyalgia rheumatica, polymyositis, dermatomyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjögren's syndrome, stiff man syndrome, systemic lupus erythematosus, Takayasu's arteritis, temporal arteritis/giant cell arteritis, ulcerative colitis, uveitis, vasculitis, vitiligo, and Wegener's granulomatosis; or hair loss diseases, in particular selected from alopecia areata, alopecia totalis, alopecia universalis, androgenic alopecia, telogen effluvium, anagen effluvium, and chemotherapy-induced alopecia.

The subject matter of the invention also concerns the use of a BMP-binding domain according to the above definition as a target for the detection or identification of RGM-binding ligands.

A further subject matter of the invention concerns the use of a BMP-binding domain according to the above definition or use of a fragment according to the above definition as an immunogen for active or passive immunization.

A further subject matter of the invention concerns a polyclonal antiserum, obtainable by immunization of a mammal with an antigenic quantity of a BMP-binding domain according to the above definition, or a polypeptide fragment according to the above definition.

The subject matter of the invention also concerns a monoclonal antibody against a BMP-binding domain according to the above definition or against a polypeptide fragment according to the above definition, or a monoclonal anti-RGM A antibody the binding of said antibody to RGM A being modulated by a BMP-binding domain as defined above, or by a polypeptide fragment thereof as defined above; or an antigen-binding fragment thereof, optionally in humanized form.

The subject matter of the invention concerns pharmaceutical agents containing a pharmaceutically acceptable carrier of at least one active component selected from:
a) A BMP-binding domain according to the above definition, a polypeptide fragment according to the above definition, or a fusion protein according to the above definition,
b) Monoclonal or polyclonal antibodies according to the above definition.

Particularly suited according to the invention are pharmaceutical agents for intrathecal, intravenous, subcutaneous, oral or parenteral, percutaneous, subdermal, intraosseal, nasal, extracorporeal, and inhalation administration.

Further pharmaceutical agents according to the invention are suited for the treatment of bone fractures, and contain at least one BMP-binding domain according to the above definition or a fusion protein according to the above definition in a liquid, semisolid, or solid carrier.

Further pharmaceutical agents according to the invention are suited for the treatment of disturbances of iron metabolism (e.g. anemia of chronic disease, juvenile hemochromatosis), and contain at least one BMP-binding domain according to the above definition or a fusion protein according to the above definition in a liquid, semisolid, or solid carrier.

A further subject matter of the invention concerns an expression vector containing at least one coding nucleic acid sequence for a BMP-binding domain according to the above definition, a fusion protein according to the above definition, or polypeptide fragment according to the above definition, operatively linked to at least one regulatory nucleic acid sequence.

The invention further relates to the following:
Recombinant microorganisms bearing at least one vector according to the above definition.
Hybridoma cell lines which produce a monoclonal antibody according to the above definition. A hybridoma cell line according to the above definition is cultivated and the produced protein product is isolated from the culture.
Method for producing a BMP-binding domain according to the above definition or a polypeptide fragment or fusion protein according to the above definition, wherein a recombinant microorganism according to the above definition is cultivated and the produced protein product is isolated from the culture.

A further subject matter of the invention is a method for producing a monoclonal antibody according to the above definition, wherein a hybridoma cell line according to the above definition is cultivated and the produced protein product is isolated from the culture.

A further subject matter of the invention concerns the use of a BMP-binding domain according to the above definition or a fusion protein according to the above definition for producing a pharmaceutical agent for stimulating an RGM receptor, in particular of neogenin, or a BMP selected from BMP-2, BMP-4, BMP-5, BMP-6, and BMP-12.

Lastly, the invention relates to the use of a monoclonal antibody according to the above definition for producing a pharmaceutical agent for blocking the activation of an RGM receptor, such as neogenin in particular.

III. Further Information for Carrying Out the Invention

1. Polypeptides

The subject matter of the invention in particular concerns BMP-binding domains of proteins of the RGM family and of peptide fragments derived from these domains. Although RGM A and its binding domains and fragments derived therefrom have been investigated according to the invention, the subject matter of the invention also concerns corresponding domains and fragments of homologous proteins, such as in particular homologous members of the RGM family, especially RGM B and RGM C.

Within the scope of the present invention, “functional equivalents” or analogs of the specifically disclosed RGM domains or polypeptides are polypeptides which differ therefrom, such as polypeptides with a degree of homology less than 100% for the BMP-binding domains of proteins according to SEQ ID NO: 2, 4, or 6, but which still have the desired biological activity. In particular, these functional equivalents should be capable of binding to at least one BMP and/or show binding in a binding test described herein, and also optionally show an inhibitory effect in a nerve fiber growth test described herein, and partially or completely inhibit nerve fiber growth with statistical significance (p<=0.05).

According to the invention, “functional equivalents” are understood in particular to be mutants which in at least one of the sequence positions of the above-referenced specific sequences contain an amino acid that is different from that specifically named, but which nevertheless has one of the biological activities named herein. “Functional equivalents” thus encompass the mutants which are obtainable through one or more amino acid additions, substitutions, deletions, and/or inversions, wherein the referenced changes may occur in any sequence position as long as they result in a mutant having the characteristics profile according to the invention. Functional equivalence in particular is present when the reactivity patterns between the mutant and the unchanged polypeptide qualitatively match; i.e., the same biological effects, for example, are observed but their level of manifestation varies greatly. The following are examples of suitable substitutions of amino acid radicals:

| Original radical | Examples of substitution |
|---|---|
| Ala | Ser |
| Arg | Lys |
| Asn | Gln; His |
| Asp | Glu |
| Cys | Ser |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

“Functional equivalents” in the above sense are also precursors of the described polypeptides, as well as functional derivatives and salts of the polypeptides. The term “salts” is understood to mean salts of carboxyl groups as well as acid addition salts of amino groups of the protein molecules according to the invention. Salts of carboxyl groups may be prepared in a manner known as such, and include inorganic salts, for example sodium, calcium, ammonium, iron, and zinc salts, as well as salts with organic bases, for example amines such as triethanolamine, arginine, lysine, piperidine, and the like. Acid addition salts, for example salts with mineral acids, such as hydrochloric acid or sulfuric acid, and salts with organic acids, such as acetic acid and oxalic acid, are likewise the subject matter of the invention. “Functional derivatives” of polypeptides according to the invention may also be provided on functional amino acid side groups or at the N- or C-terminal ends thereof, using known techniques. Such derivatives include, for example, aliphatic esters of carboxylic acid groups, amides of carboxylic acid groups obtainable by reaction with ammonia or with primary or secondary amine; N-acyl derivatives of free amino groups, prepared by reaction with acyl groups; or O-acyl derivatives of free hydroxy groups, prepared by reaction with acyl groups.

Of course, “functional equivalents” also include polypeptides which are available from other organisms, as well as naturally occurring variants. For example, regions of homologous sequence ranges may be ascertained by sequence comparison, and equivalent enzymes may be determined according to the specific requirements of the invention.

“Functional equivalents” are also fusion proteins having one of the above-referenced polypeptide sequences or functional equivalents derived therefrom, and at least one additional, different heterologous sequence in a functional N- or C-terminal linkage (i.e., significant mutual functional impairment of the fusion protein portions). Nonlimiting examples of such heterologous sequences include enzymes and immunoglobulins.

“Functional equivalents” encompassed according to the invention include homologues to the specifically disclosed proteins, i.e., peptides. These functional equivalents have at least 40% or at least 50%, or at least 60%, for example 75% or in particular at least 85%, for example 90%, 95%, or 99%, homology to one of the specifically disclosed sequences, for example calculated according to the algorithm developed by Pearson and Lipman, Proc. Natl. Acad, Sci. (USA) 85(8), 1988, 2444-2448. A percent homology of a homologous polypeptide according to the invention means in particular a percent identity of the amino acid radicals relative to the total length of one of the amino acid sequences specifically described herein.

Unless indicated otherwise, according to the invention a “derived” amino acid sequence means a sequence having an initial sequence with an identity of at least 80% or at least 90%, in particular 91%, 92%, 93%, 94%, 95%, 96%, 97%, 98%, and 99%.

“Identity” or “homology” between two sequences means the identity of the amino acid radicals over the respective entire sequence length, for example the identity which is calculated by comparison, using Vector NTI Suite 7.1 software from InforMax (USA), applying the Clustal method (Higgins D. G. and Sharp, P. M., Fast and sensitive multiple sequence alignments on a microcomputer, Comput. Appl. Biosci. April 1989, 5(2): 151-1), with the following parameter settings:

Multiple Alignment Parameter:

| | |
|---|---|
| Gap opening penalty | 10 |
| Gap extension penalty | 10 |
| Gap separation penalty range | 8 |
| Gap separation penalty | off |
| % identity for alignment delay | 40 |
| Residue specific gaps | off |
| Hydrophilic residue gap | off |
| Transition weighing | 0 |

Pairwise Alignment Parameter:

| FAST algorithm | on |
|---|---|
| K-tuple size | 1 |
| Gap penalty | 3 |
| Window size | 5 |
| Number of best diagonals | 5 |

In the event of possible protein glycosylation, the equivalents encompass proteins of the type described above in deglycosylated or glycosylated form, and in modified forms which may be obtained by changing the glycosylation pattern.

Homologues of the peptides according to the invention may be identified by screening combinatorial banks of mutants, for example truncated mutants. For example, a variegated bank of peptide variants may be created by combinatorial mutagenesis at the nucleic acid level, such as by enzymatic ligation of a mixture of synthetic oligonucleotides. There are numerous methods which may be used to create banks of potential homologues from a degenerate oligonucleotide sequence. The chemical synthesis of a degenerate gene sequence may be carried out in a DNA synthesizer, and the synthetic gene may then be ligated in a suitable expression vector. Use of a degenerate gene set allows all sequences which code the desired set to potential protein sequences to be prepared in a mixture. Methods for synthesizing degenerate oligonucleotides are known to one skilled in the art (for example, Narang, S. A. (1983) Tetrahedron 39: 3; Itakura et al. (1984) Annu. Rev. Biochem. 53: 323; Itakura et al. (1984) Science 198: 1056; Ike et al. (1983) Nucleic Acids Res. 11:477).

2. Nucleic Acids

A further subject matter of the invention concerns the coding nucleic acid sequences for the above-described BMP-binding domains and polypeptides, such as in particular according to SEQ ID NO: 1, 3, and 5, as well as nucleic acid sequences or partial sequences derived therefrom which code for the above-described peptide fragments.

All of the nucleic acid sequences according to the invention (single- and double-stranded DNA and RNA sequences, for example cDNA and mRNA) are known as such through chemical synthesis from the nucleotide structural units, for example by fragment condensation of individual overlapping, complementary nucleic acid structural units of the double helix. The chemical synthesis of oligonucleotides may be carried out in a known manner, for example according to the phosphoramidite method (Voet, Voet, 2nd Ed., Wiley Press New York, 896-897). The addition of synthetic oligonucleotides and filling of gaps using the Klenow fragment of DNA polymerase and ligation reactions as well as general cloning methods are described in Sambrook et al. (1989), Molecular Cloning: A Laboratory Manual, Cold Spring Harbor Laboratory Press.

Unless indicated otherwise, according to the invention a “derived” nucleic acid sequence means a sequence whose initial sequence has an identity of at least 80% or at least 90%, in particular 91%, 92%, 93%, 94%, 95%, 96%, 97%, 98%, and 99%.

“Identity” between two nucleic acids means the identity of the nucleotide over the entire respective length of the nucleic acid, in particular the identity which [is calculated] by comparison, using Vector NTI Suite 7.1 software from InforMax (USA), applying the Clustal method (see above).

The subject matter of the invention also concerns nucleic acid sequences which code for one of the above peptides and functional equivalents thereof, and which are obtainable, for example, using synthetic nucleotide analogs.

The invention relates to isolated nucleic acid molecules which code for peptides or biologically active segments thereof according to the invention, as well as nucleic acid fragments which may be used, for example, as hybridization samples or primers for identifying or amplifying coding nucleic acids according to the invention.

The nucleic acid molecules according to the invention may also contain untranslated sequences of the 3′ and/or 5′ end of the coding gene region.

An “isolated” nucleic acid molecule is separated from other nucleic acid molecules which are present in the natural source of the nucleic acid, and may also be essentially free of other cellular material or culture medium if it is produced using recombinant techniques, or may be free of chemical precursors or other chemicals if it is chemically synthesized.

A nucleic acid molecule according to the invention may be isolated using standard techniques of molecular biology and the sequence information provided according to the invention. For example, cDNA may be isolated from a suitable cDNA bank by using one of the specifically disclosed complete sequences or a segment thereof as hybridization sample, and using standard hybridization techniques (such as those described, for example, in Sambrook, J., Fritsch, E. F., and Maniatis, T., Molecular Cloning: A Laboratory Manual, 2nd Ed., Cold Spring Harbor Laboratory, Cold Spring Harbor Laboratory Press, Cold Spring Harbor, N.Y., 1989). In addition, a nucleic acid molecule which includes one of the sequences according to the invention or a segment thereof may be isolated by a polymerase chain reaction, using the oligonucleotide primer which was produced on the basis of this sequence. The nucleic acid amplified in this manner may be cloned in a suitable vector and characterized by DNA sequence analysis. The oligonucleotides according to the invention may also be produced using standard synthesis methods, for example using a DNA synthesizer.

The invention also encompasses the complementary nucleic acid molecules or a segment thereof which are complementary to the specifically described nucleotide sequences.

The nucleotide sequences according to the invention allow production of samples and primers which may be used for identification and/or cloning of homologous sequences in other cell types and organisms. Such samples or primers typically include a nucleotide sequence range which hybridizes under stringent conditions at at least approximately 12, preferably at least approximately 25, for example approximately 40, 50, or 75 successive nucleotides of a sense strand of a nucleic acid sequence according to the invention or a corresponding antisense strand.

Other nucleic acid sequences according to the invention are derived from coding sequences for the RGM domains and peptides according to the invention, and differ therefrom by virtue of addition, substitution, insertion, or deletion of individual or multiple nucleotides, but continue to code for peptides having the desired characteristics profile.

Also encompassed according to the invention are nucleic acid sequences which include so-called silent mutations, or which are modified in comparison to a specifically named sequence, corresponding to the codon use of a specialized organism of origin or host organism, as well as naturally occurring variants, for example splice variants or allele variants. The subject matter of the invention also concerns sequences obtainable by conservative nucleotide substitutions (i.e., replacement of the amino acid in question by an amino acid of the same charge, size, polarity, and/or solubility).

The subject matter of the invention also concerns molecules derived from the specifically disclosed nucleic acids by sequence polymorphisms. These genetic polymorphisms may exist among individuals within a population due to natural variation. These natural variations typically result in a variance of 1 to 5% in the nucleotide sequence of a gene.

The invention also encompasses nucleic acid sequences which hybridize with the above-referenced coding sequences or are complementary thereto. These polynucleotides may be located by sampling genomic or cDNA banks, and may optionally be propagated by PCR, using suitable primers, and then isolated therefrom, using suitable samples. Another possibility is the transformation of suitable microorganisms using polynucleotides or vectors according to the invention, propagation of the microorganisms and thus of the polynucleotides, and subsequent isolation thereof. Polynucleotides according to the invention may also be chemically synthesized.

The property of being able to “hybridize” polynucleotides means the capability of a poly- or oligonucleotide to bind to an essentially complementary sequence under stringent conditions, while nonspecific binding between noncomplementary partners does not occur under these conditions. To this end, the sequences should be 70-100%, in particular 90-100%, for example 95%, 96%, 97%, 98%, or 99% complementary. The property of complementary sequences to specifically bind to one another is employed, for example, in the Northern or Southern blot technique, or for primer binding in PCR or RT-PCR.

Oligonucleotides beginning at a length of 30 base pairs are usually used for this purpose. “Under stringent conditions,” for example in the Northern blot technique, means the use of a wash solution, for example 0.1×SSC buffer with 0.1% SDS (20×SSC: 3 M NaCl, 0.3 M Na citrate, pH 7.0) at a temperature of 50-70° C., preferably 60-65° C., such as for elution of nonspecifically hybridized cDNA samples or oligonucleotides. As previously mentioned, only nucleic acids which are complementary to a high degree remain bound to one another. The setting of stringent conditions is known to one skilled in the art and is described, for example, in Ausubel et al., Current Protocols in Molecular Biology, John Wiley & Sons, N.Y. (1989), 6.3.1-6.3.6.

A further aspect of the invention relates to “antisense” nucleic acids. Antisense nucleic acids include a nucleotide sequence that is complementary to a coding “sense” nucleic acid. The antisense nucleic acid may be complementary to the entire coding strand, or to only a segment thereof. In a further embodiment, the antisense nucleic acid molecule is antisense to a noncoding region of the coding strand of a nucleotide sequence. The term “noncoding region” relates to the sequence segments referred to as 5′- and 3′-untranslated regions.

An antisense oligonucleotide may have a length, for example, of approximately 5, 10, 15, 20, 25, 30, 35, 40, 45, or 50 nucleotides. An antisense nucleic acid according to the invention may be constructed by chemical synthesis and enzymatic ligation reactions, using methods known in the technical field. An antisense nucleic acid may be chemically synthesized, using naturally occurring nucleotides or variously modified nucleotides having a configuration such that they increase the biological stability of the molecules or increase the physical stability of the duplex created between the antisense and the sense nucleic acid. Phosphorthioate derivatives and acridin-substituted nucleotides, for example, may be used. Examples of modified nucleotides which may be used to create the antisense nucleic acid include 5-fluorouracil, 5-bromouracil, 5-chlorouracil, 5-iodouracil, hypoxanthin, xanthin, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N-2-carboxypropyl)uracil, (acp3)w, and 2,6-diaminopurine. The antisense nucleic acid may also be biologically created by using an expression vector in which a nucleic acid has been subcloned in the antisense direction.

3. Expression Constructs and Vectors

A further subject matter of the invention concerns expression constructs containing a nucleic acid sequence, under the genetic control of regulatory nucleic acid sequences, which codes for an RGM peptide according to the invention or functional equivalent, or immunoglobulin; as well as vectors including at least one of these expression constructs.

Such constructs according to the invention preferably include a promoter at 5′ upstream from the particular coding sequence, and a terminator sequence and optionally other common regulatory elements at 3′ downstream, and specifically, each being operatively linked to the coding sequence. An “operative linkage” means the sequential placement of promoter, coding sequence, terminator, and optionally other regulatory elements such that each of the regulatory elements is able to properly fulfill its function in the expression of the coding sequence. Examples of operatively linkable sequences are targeting sequences as well as enhancers, polyadenylation signals, and the like. Further regulatory elements include selectable markers, amplification signals, replication origins, and the like. Suitable regulatory sequences are described, for example, in Goeddel, Gene Expression Technology: Methods in Enzymology 185, Academic Press, San Diego, Calif. (1990).

In addition to the artificial regulation sequences, the natural regulation sequence may be present before the actual structural gene. This natural regulation may be eliminated, if needed, by genetic modification, and the expression of the genes increased or decreased. However, the gene construct may also have a simpler structure; i.e., no additional regulation signals are inserted in front of the structural gene, and the natural promoter together with its regulation is not removed. Instead, the natural regulation sequence is mutated in such a way that regulation no longer occurs, and the gene expression is increased or decreased. The nucleic acid sequences may be contained in one or more copies in the gene construct.

The following are examples of promoters which may be used: cos, tac, trp, tet, trptet, lpp, lac, lpplac, laclq, T7, T5, T3, gal, trc, ara, SP6, lambda PR, or lambda PL promoter, which are advantageously used in gram-negative bacteria; in addition to the amy and SPO2 gram-positive promoters, the ADC1, MFalpha, AC, P-60, CYC1, or GAPDH yeast promoters, the plant promoters CaMV/35S, SSU, OCS, lib4, usp, STLS1, B33, or not plant promoters, or the ubiquitin or phaseolin promoter. The use of inducible promoters, for example light- and in particular temperature-inducible promoters such as the PᵣPₗ promoter, is particularly preferred. In principle, all natural promoters together with their regulation sequences may be used. Synthetic promoters may also be advantageously used.

The referenced regulatory sequences are intended to permit the targeted expression of the nucleic acid sequences and protein expression. Depending on the host organism, this may mean, for example, that the gene is expressed, or expressed only after induction, or that the gene is immediately expressed and/or overexpressed.

The regulatory sequences or factors may preferably positively influence the expression, thus increasing or decreasing same. Thus, amplification of the regulatory elements may advantageously take place at the transcription level by using strong transcription signals as promoters and/or “enhancers.” However, it is also possible to amplify the translation, for example by enhancing the stability of the mRNA.

An expression cassette is produced by fusion of a suitable promoter with a suitable coding nucleotide sequence and a terminator or polyadenylation signal. To this end, common recombination and cloning techniques are used as described, for example, in T. Maniatis, E. F. Fritsch, and J. Sambrook, Molecular Cloning: A Laboratory Manual, Cold Spring Harbor Laboratory, Cold Spring Harbor, N.Y. (1989), and in T. J. Silhavy, M. L. Berman, and L. W. Enquist, Experiments with Gene Fusions, Cold Spring Harbor Laboratory, Cold Spring Harbor, N.Y. (1984), and in Ausubel, F. M. et al., Current Protocols in Molecular Biology, Greene Publishing Assoc. and Wiley Interscience (1987).

For expression in a suitable host organism, the recombinant nucleic acid construct, i.e., gene construct, is advantageously inserted into a host-specific vector which permits optimal expression of the genes in the host. Vectors are well known to one skilled in the art, and are described, for example, in Cloning Vectors (Pouwels, P. H. et al., Eds., Elsevier, Amsterdam-New York-Oxford, 1985). Besides plasmids, vectors are understood to mean all other vectors known to one skilled in the art, for example phages, viruses such as SV40, CMV, baculovirus, and adenovirus, transposons, IS elements, phasmids, cosmids, and linear or circular DNA. These vectors may be autonomously replicated in the host organism or chromosomally replicated.

The following are examples of suitable expression vectors:

Common fusion expression vectors, such as pGEX (Pharmacia Biotech Inc; Smith, D. B. and Johnson, K. S. (1988) Gene 67: 31-40), pMAL (New England Biolabs, Beverly, Mass.), and pRIT 5 (Pharmacia, Piscataway, N.J.), in which glutathione S-transferase (GST), maltose E binding protein, or protein A is fused to the recombinant target protein.

Non-fusion protein expression vectors such as pTrc (Amann et al. (1988) Gene 69: 301-315) and pET 11d (Studier et al., Gene Expression Technology: Methods in Enzymology 185, Academic Press, San Diego, Calif. (1990), 60-89).

Yeast expression vectors for expression in S. cerevisiae yeast, such as pYepSec1 (Baldari et al. (1987) Embo J. 6: 229-234), pMFa (Kurjan and Herskowitz (1982) Cell 30: 933-943), pJRY88 (Schultz et al. (1987) Gene 54: 113-123), and pYES2 (Invitrogen Corporation, San Diego, Calif.). Vectors and methods for constructing vectors which are suitable for use in other fungi, such as filamentous fungi, include those described in detail in van den Hondel, C. A. M. J. J. & Punt, P. J. (1991) “Gene transfer systems and vector development for filamentous fungi,” in Applied Molecular Genetics of Fungi, J. F. Peberdy et al., Eds., 1-28, Cambridge University Press: Cambridge.

Baculovirus vectors, which are available for expression of proteins in cultured insect cells (Sf9 cells, for example) include the pAc series (Smith et al. (1983) Mol. Cell Biol. 3: 2156-2165) and the pVL series (Lucklow and Summers (1989) Virology 170: 31-39).

Plant expression vectors, such as those described in detail in Becker, D., Kemper, E., Schell, J., and Masterson, R. (1992), “New plant binary vectors with selectable markers located proximal to the left border,” Plant Mol. Biol. 20: 1195-1197; and Bevan, M. W. (1984), “Binary agrobacterium vectors for plant transformation,” Nucl. Acids Res. 12: 8711-8721.

Mammalian expression vectors, such as pCDM8 (Seed, B. (1987), Nature 329: 840) and pMT2PC (Kaufman et al. (1987), EMBO J. 6: 187-195).

Further suitable expression systems for prokaryotic and eukaryotic cells are described in Chapters 16 and 17 of Sambrook, J., Fritsch, E. F., and Maniatis, T., Molecular Cloning: A Laboratory Manual, 2nd Ed., Cold Spring Harbor Laboratory, Cold Spring Harbor Laboratory Press, Cold Spring Harbor, N.Y., 1989.

4. Recombinant Host Organisms

By use of the vectors according to the invention, recombinant organisms may be produced which, for example, are transformed using at least one vector according to the invention and may be used for producing the domains or polypeptides according to the invention. The above-described recombinant constructs according to the invention are advantageously introduced into a suitable host system and expressed. Preferably used are common cloning and transfection methods known to one skilled in the art, for example coprecipitation, protoplast fusion, electroporation, retroviral transfection, and the like, in order to bring the referenced nucleic acids to expression in the particular expression system. Suitable systems are described, for example, in Current Protocols in Molecular Biology, F. Ausubel et al., Eds. Wiley Interscience, New York 1997, or Sambrook et al., Molecular Cloning: A Laboratory Manual, 2nd Ed., Cold Spring Harbor Laboratory, Cold Spring Harbor Laboratory Press, Cold Spring Harbor, N.Y., 1989.

In principle, all organisms are suitable as host organisms which permit expression of the nucleic acids according to the invention, their allele variants, functional equivalents, or derivatives thereof. “Host organisms” are understood to mean bacteria, fungi, yeasts, or plant or animal cells, for example. Preferred organisms are bacteria, such as of the genus Escherichia, for example Escherichia coli, Streptomyces, Bacillus, or Pseudomonas, eukaryotic microorganisms such as Saccharomyces cerevisiae or Aspergillus, and higher eukaryotic cells from animals or plants, for example, Sf9, CHO, or HEK293 cells.

Successfully transformed organisms may be selected by use of marker genes, which are likewise contained in the vector or in the expression cassette. Examples of such marker genes are genes for antibiotic resistance and for enzymes which catalyze a chromophoric reaction and cause staining of the transformed cell. These marker genes may then be selected, using automatic cell sorting. Microorganisms successfully transformed by a vector and which carry a corresponding antibiotic resistance gene (G418 or hygromycin, for example) may be selected using appropriate antibiotic-containing media or culture media. Marker proteins which are presented at the cell surface may be used for selection by means of affinity chromatography.

If desired, the gene product may also be brought to expression in transgenic organisms, such as transgenic animals, in particular mice and sheep, or transgenic plants.

A further subject matter of the invention concerns methods for recombinant production of RGM domains or polypeptides according to the invention or functional, biologically active fragments thereof, wherein a peptide-producing recombinant host organism is cultivated, and optionally the expression of the polypeptides is induced, and these polypeptides are isolated from the culture. The peptides may also be produced in this manner on a commercial scale, if desired.

The recombinant host may be cultivated and fermented according to known methods. Bacteria may be propagated, for example, in TB or LB medium and at a temperature of 20 to 40° C. and a pH of 6 to 9. Suitable cultivation conditions are described in detail in T. Maniatis, E. F. Fritsch, and J. Sambrook, Molecular Cloning: A Laboratory Manual, Cold Spring Harbor Laboratory, Cold Spring Harbor, N.Y. (1989), for example.

If the polypeptides are not secreted into the culture medium, the cells are then macerated and the product is harvested from the lysate, using known protein isolation methods. The cells may optionally be macerated using high-frequency ultrasound, high pressure, for example using a French pressure cell, by osmolysis, through the action of detergents, lytic enzymes or organic solvents, by homogenization, or by a combination of several of the listed methods.

The peptides may be purified using known chromatographic methods, for example molecular sieve chromatography (gel filtration), such as Q sepharose chromatography, ion exchange chromatography and hydrophobic chromatography, and by using other common methods such as ultrafiltration, crystallization, salting out, dialysis, and native gel electrophoresis. Suitable methods are described, for example, in Cooper, F. G., Biochemische Arbeitsmethoden [Biochemical Procedural Methods], Verlag Walter de Gruyter, Berlin, New York, or in Scopes, R., Protein Purification, Springer Verlag, New York, Heidelberg, Berlin.

For isolation of the recombinant peptide it is particularly suitable to use vector systems or oligonucleotides which extend the cDNA by given nucleotide sequences and thus code for modified polypeptides or fusion proteins, which are used, for example, for simpler purification. So-called “tags” which function as anchors represent an example of suitable modifications, for example the modification known as the hexahistidine anchor, or epitopes which may be identified as antigens of antibodies (described, for example, in Harlow, E. and Lane, D., 1988, Antibodies: A Laboratory Manual, Cold Spring Harbor Press (New York)). These anchors may be used to attach the peptides to a fixed substrate, such as a polymer matrix, which may be filled into a chromatographic column, for example, or used on a microtiter plate or some other substrate.

At the same time, these anchors may be used for identification of the peptides. For identification of the peptides, common markers such as fluorescent dyes, enzyme markers which form a detectable reaction product after reaction with a substrate, or radioactive markers, may be used, alone or in combination with the anchors, for derivatization of the peptides.

5. Immunoglobulins

5.1 Definitions

The subject matter of the present invention concerns monoclonal or polyclonal antibodies which bind specifically to an RGM protein according to the invention or derivative/equivalent thereof, i.e., antibodies with specificity for an RGM protein according to the invention or derivative/equivalent thereof. The subject matter of the present invention further concerns portions of these antibodies, in particular antigen-binding portions thereof, i.e., antibody fragments which bind an RGM protein according to the invention or derivative/equivalent thereof.

The antibody according to the invention is preferably selected in such a way that it has given binding kinetics (for example, high affinity, low dissociation, low off-speed (k_{off}), strong neutralizing activity) for the specific binding to RGM protein according to the invention or derivative/equivalent thereof.

Thus, antibodies having an affinity in the range of K_{D}=10⁻⁶-10⁻¹² M for the RGM protein according to the invention or derivative/equivalent thereof may be provided.

According to a further aspect, the antibodies according to the invention may be selected so that they bind the RGM protein or derivative/equivalent thereof at a k_{off} speed constant of 0.1 s⁻¹ or less.

The antibodies are preferably isolated antibodies. According to a further aspect, the antibodies are neutralizing antibodies. The antibodies according to the invention include in particular monoclonal and recombinant antibodies. The antibody according to the invention may contain an amino acid sequence which originates entirely from a single species; thus, for example, a human antibody may be a rat antibody or a mouse antibody. According to further embodiments, the antibody may be a chimeric antibody or a CDR graft antibody or other form of a humanized antibody.

The term “antibody” refers to immunoglobulin molecules formed from four polypeptide chains, two heavy (H) chains and two light (L) chains. The chains are generally linked to one another by disulfide bonds. Each heavy chain is composed of a variable region of the chain (abbreviated herein as HCVR or VH) and a constant region of the heavy chain. The constant region of the heavy chain is formed from three domains CH1, CH2, and CH3. Each light chain is composed of a variable region of the light chain (abbreviated herein as LCVR or VL) and a constant region of the light chain. The constant region of the light chain is formed from a CL domain. The VH and VL regions may be further divided into hypervariable regions, referred to as complementarity-determining regions (CDR), and interspersed with conserved regions, referred to as framework (FR) regions. Each VH and VL region is formed from three CDRs and four FRs, positioned in the following sequence from the N terminus to the C terminus: FR1, CDR1, FR2, CDR2, FR3, CDR3, FR4.

The term “antigen-binding portion” of an antibody (or simply “antibody portion”) refers to one or more fragments of an antibody with specificity for an RGM protein according to the invention or derivative/equivalent thereof, wherein the fragment or fragments are still able to specifically bind the RGM protein or derivative/equivalent thereof. It has been shown that the antigen-binding function of an antibody may be ascertained from fragments of a complete antibody. Examples of binding fragments in the sense of the term “antigen-binding portion” of an antibody include (i) an Fab fragment, i.e., a monovalent fragment composed of the VL, VH, CL, and CH1 domains; (ii) an F(ab′)₂ fragment, i.e., a bivalent fragment which contains two Fab fragments linked to one another in the hinge region via a disulfide bridge; (iii) an Fd fragment composed of the VH and CH1 domains; (iv) an Fv fragment composed of the VL and VH domains of a single arm of an antibody; (v) a dAb fragment (Ward et al. (1989) Nature 341: 544-546) composed of a VH domain or VH, CH1, CH2, DH3, or VH, CH2, CH3; and (vi) an isolated complementarity-determining region (CDR). Although the two domains of the Fv fragment, namely, VL and VH, are coded by different genes, they may be joined together using a synthetic linker according to recombinant methods, by means of which they may be produced as a single protein chain in which the VL and VH regions meet to form monovalent molecules (known as single-chain Fv (ScFv); see, for example, Bird et al. (1988) Science 242: 423-426; and Huston et al. (1988) Proc. Natl. Acad. Sci. USA 85: 5879-5883). Such single-chain antibodies are also encompassed within the term “antigen-binding portion” of an antibody. Other forms of single-chain antibodies such as “diabodies” are likewise included. Diabodies are bivalent, bispecific antibodies in which VH and VL domains are expressed on a single polypeptide chain, except that a linker is used which is too short to allow the two domains to meet on the same chain, thereby forcing the domains to pair with complementary domains of a different chain and to form two antigen-binding sites (see, for example, Holliger, P. et al. (1993) Proc. Natl. Acad. Sci. USA 90: 6444-6448; Poljak, R. J. et al. (1994) Structure 2: 1121-1123).

Furthermore, an antibody or antigen-binding portion thereof may be part of a larger immunoadhesin molecule, which is formed by covalent or noncovalent association of the antibody or antibody portion with one or more additional proteins or peptides. Such immunoadhesin molecules include the use of the streptavidin core region to produce a tetrameric scFv molecule (Kipriyanov, S. M. et al. (1995) “Human antibodies and hybridomas” 6: 93-101) and the use of a cystein radical, a marker peptide, and a C-terminal polyhistidine tag, to produce bivalent and biotinylated scFv molecules (Kipriyanov, S. M. et al. (1994) Mol. Immunol. 31: 1047-1058).

Antibody portions, such as Fab and F(ab′)₂ fragments, may be produced from entire antibodies by using conventional techniques such as digestion using papain or pepsin. Antibodies, antibody portions, and immunoadhesin molecules may also be obtained by using standardized recombinant DNA techniques. An “isolated antibody with specificity for an RGM protein according to the invention or derivative/equivalent thereof” encompasses an antibody with specificity for an RGM protein according to the invention or derivative/equivalent thereof which is essentially free of other antibodies having different antigen specificities.

The term “neutralizing antibody” describes an antibody whose binding to a given antigen results in inhibition of the biological activity of the antigen. This inhibition of the biological activity of the antigen may be assessed by measuring one or more indicators for the biological activity of the antigen, using a suitable in vitro or in vivo assay.

The term “monoclonal antibody” describes an antibody which originates from a hybridoma (for example, an antibody that is secreted by a hybridoma produced using hybridoma technology, such as the standardized hybridoma methodology according to Köhler and Milstein). An antibody having specificity for an RGM protein according to the invention or derivative/equivalent thereof and which originates from a hybridoma is therefore referred to as a monoclonal antibody.

The term “recombinant antibody” describes antibodies which are expressed, produced, or isolated using recombinant means, such as antibodies which are expressed using a recombinant expression vector that is transfected into a host cell; antibodies which are isolated from a recombinant combinatorial antibody bank; antibodies which are isolated from an animal (a mouse, for example) which is made transgenic using human immunoglobulin genes (see, for example, Taylor, L. D. et al. (1992) Nucl. Acids Res. 20: 6287-6295); or antibodies expressed, produced, or isolated in some other manner in which the given immunoglobulin gene sequences (such as human immunoglobulin gene sequences) are combined with other DNA sequences. Recombinant antibodies include, for example, chimeric, CDR graft, and humanized antibodies.

The term “human antibody” describes antibodies whose variable and constant regions correspond to or originate from immunoglobulin sequences of the human germline, such as described, for example, by Kabat et al. (see Kabat et al. (1991) Sequences of Proteins of Immunological Interest, Fifth Edition, U.S. Department of Health and Human Services, NIH Publication No. 91-3242). However, the human antibodies according to the invention may contain amino acid radicals which are not coded by human germline immunoglobulin sequences (for example, mutations which are introduced by random or site-specific mutagenesis in vitro or by somatic mutation in vivo), for example in the CDRs, and in particular in CDR3. Recombinant human antibodies according to the invention have variable regions, and may also contain constant regions which originate from immunoglobulin sequences of the human germline (see Kabat, E. A. et al. (1991) Sequences of Proteins of Immunological Interest, Fifth Edition, U.S. Department of Health and Human Services, NIH Publication No. 91-3242). According to certain embodiments, however, such recombinant human antibodies are subjected to in vitro mutagenesis (or, if an animal is used that is made transgenic using human Ig sequences, subjected to a somatic in vivo mutagenesis), so that the amino acid sequences of the VH and VL regions of the recombinant antibody are sequences which by nature do not exist within the human antibody germline repertoire in vivo, even though they are related to or originate from VH and VL sequences of the human germline. According to certain embodiments, such recombinant antibodies are the result of a selective mutagenesis or back mutation, or both.

The term “back mutation” refers to a method in which some or all of the somatically mutated amino acids of a human antibody are replaced by the corresponding germline residues of a homologous germline antibody sequence. The sequences for the heavy and light chain of a human antibody according to the invention are compared separately to the germline sequences in the VBASE database in order to identify the sequences with the greatest homology. Differences in the human antibody according to the invention are attributed to the germline sequence by mutating at defined nucleotide positions which code such differing amino acids. The direct or indirect importance of each amino acid, identified in this manner as a candidate for a back mutation, for the antigen binding should be investigated, and an amino acid which after mutation impairs a desirable characteristic of the human antibody should not be included in the final human antibody. To keep the number of amino acids for a back mutation as small as possible, the amino acid positions which, although they are different from the next germline sequence are identical to the corresponding amino acid sequence of a second germline, may remain unchanged, provided that the second germline sequence is identical to and colinear with the sequence of the human antibody according to the invention, at least with respect to 10 and preferably with respect to 12 amino acids on both sides of the amino acid in question. Back mutations may be performed at any given stage in the antibody optimization.

The term “chimeric antibody” encompasses antibodies in which individual portions of the molecule are derived from different species. Thus, chimeric antibodies, without being limited thereto, are, for example, antibodies which contain the sequences for the variable region of the heavy and light chains from one species, in which, however, the sequences of one or more of the CDR-regions from VH and/or VL are replaced by CDR sequences of another species. Such antibodies may contain the variable regions of the heavy and light chains from a mouse, in which one or more of the mouse CDRs (CDR3, for example) are replaced by human CDR sequences.

The term “humanized antibody” describes antibodies which contain sequences of the variable region of the heavy and light chains from a nonhuman species (for example, mouse, rat, rabbit, chicken, camelids, goats), in which, however, at least one portion of the VH and/or VL sequence has been modified to be “human-like,” i.e., to be more like variable sequences in the human germline. One type of humanized antibody is a CDR graft antibody, in which the corresponding nonhuman CDR sequences are replaced by inserting human CDR sequences into nonhuman VH and VL sequences.

One method for measuring the binding kinetics of an antibody is based on the so-called surface plasmon resonance. The term “surface plasmon resonance” refers to an optical phenomenon which allows biospecific interactions to be analyzed by detecting changes in protein concentrations by means of a biosensor matrix, using the Biacore system, for example (Pharmacia Biosensor AB, Uppsala, Sweden and Piscataway, N.J.). For further information, see Jönsson, U. et al. (1993) Ann. Biol. Clin. 51: 19-26; Jönsson, U. et al., (1991) Biotechniques 11: 620-627; Johnsson, B. et al. (1995) J. Mol. Recognit. 8: 125-131; and Johnnson, B. et al. (1991) Anal. Biochem. 198: 268-277.

The term “K_{off}” describes the off-speed constant for the dissociation of an antibody from the antibody/antigen complex.

The term “K_{d}” describes the dissociation constant of a given antibody-antigen interaction.

The binding affinity of the antibody according to the invention may be evaluated by using standardized in vitro immunoassays, such as ELISA or Biacore analyses.

5.2 Production of Immunoglobulins

5.2.1 Production of Polyclonal Antibodies

The present invention relates to polyclonal antibodies directed against RGM domains and polypeptides according to the invention, and production of same.

For this purpose, a host having at least one RGM protein according to the invention or derivative/equivalent is immunized, and an antibody-containing serum as a response to the immunization is recovered from the host.

If the RGM polypeptides to be used are not immunogenic or are only weakly immunogenic, their immunogenicity may be increased by coupling them to carriers, preferably a carrier protein such as keyhole limpet hemocyanin (KLH), limulus polyphemus hemocyanin (LPH), bovine serum albumin (BSA), or ovalbumin (OVA). A number of coupling options which are generally known are available to one skilled in the art. The reaction with glutardialdehyde, for example, may be practical, for example by incubating RGM protein with a suitable peptide or peptide mixture in water or an aqueous solvent. This reaction may conveniently be carried out at ambient temperature, i.e., as a rule at room temperature. However, it may be useful to perform cooling or slight heating. The reaction generally provides the desired result within a few hours, a reaction period of 2 hours, for example, being in the typical range. The glutardialdehyde concentration is generally in the ppm to % range, advantageously from 10 ppm to 1%, preferably from 100 ppm to 0.5%. Optimization of the reaction parameters is within the skill of one skilled in the art.

In addition to the antigen, the compositions generally contain further auxiliary substances, in particular adjuvants commonly used for immunization, for example Freund's adjuvant. In particular, complete Freund's adjuvant is used for the first immunization, whereas all further immunizations are carried out using incomplete Freund's adjuvant. The immunization cocktails are produced by adding the antigen (immunogen), preferably in the form of the above-described component mixture, to the auxiliary substance(s). As a rule the antigen is emulsified.

Rodents or rabbits are particularly suited as host. The immunization cocktails are injected, preferably subcutaneously, into these or other suitable hosts. The antibody titers may be determined using immunoassay, for example competitively using a sheep antiserum directed against the host IgG and marked RGM protein. Thus, at the end of the immunization a decision may be made as to whether a given host is suitable for antibody recovery. If, for example, four immunizations are carried out, the antibody titer may be determined after the third immunization and antibodies may then be recovered from animals having an adequate antibody titer.

For recovery of produced antibody, blood is preferably withdrawn from the hosts over several weeks or months. The host may then be exsanguinated. Serum containing the desired antibody may be harvested in a manner known as such from the blood thus recovered. The full serum thus obtained may be further purified, if necessary, in a manner known to one skilled in the art in order to enrich the antibody fraction and in particular the RGM protein-recognizing antibody contained therein.

According to one particular embodiment of this method, at least one antibody is selected from the serum which specifically recognizes the RGM protein used as immunogen or a derivative/equivalent thereof. In this context, “specificity” means a higher binding affinity of the antibody for the immunogen than for other proteins which in particular are immunogen-related.

5.2.2 Production of Monoclonal Antibodies

Immunoglobulins which may be used according to the invention are obtainable by methods known as such. Thus, the hybridoma technology allows monospecific antibodies to be produced for an antigen of interest. Furthermore, recombinant antibody techniques such as in vitro screening of antibody banks have been developed by means of which such specific antibodies may likewise be produced.

Thus, for example, an animal having the antigen of interest may be immunized. This in vivo approach may also include establishing a series of hybridomas from the lymphocytes or spleen cells of an animal and selecting a hybridoma which secretes the one antibody which specifically binds the antigen. The animal to be immunized may be, for example, a mouse, rat, rabbit, chicken, camelid, or sheep, or may be a transgenic version of the animals referenced above, for example a transgenic mouse with human immunoglobulin genes which produces human antibodies following an antigenic stimulus. Other types of animals which may be immunized include mice with severe combined immunodeficiency (SCID) which have been reconstituted using human peripheral mononuclear blood cells (chimeric hu-PBMC-SCID mice) or using lymphoid cells or precursors thereof, as well as mice which have been administered a lethal dose of total-body radiation, then protected against radiation using bone marrow cells from mice with severe combined immunodeficiency (SCID), and then receiving a transplant of functional human lymphocytes (the so-called Trimera system). A further type of animal to be immunized is an animal (a mouse, for example) in whose genome an endogenous gene which codes the antigen of interest has been eliminated (“knocked out”), for example by homologous recombination, so that after immunization with the antigen this animal identifies the antigen as foreign. It is clear to one skilled in the art that the polyclonal or monoclonal antibodies produced according to this method are characterized and selected by using known screening methods, including ELISA techniques, without, however, being limited thereto.

According to a further embodiment, a recombinant antibody bank is screened using the antigen. The recombinant antibody bank may be expressed, for example, on the surface of bacteriophages, on the surface of yeast cells, or on the surface of bacterial cells. The recombinant antibody bank may be an scFv bank or an Fab bank, for example. According to a further embodiment, antibody banks may be expressed as RNA protein fusions.

A further approach for producing antibodies according to the invention comprises a combination of in vivo and in vitro approaches. For example, the antigen may act on the antibody repertoire by immunizing an animal with the antigen in vivo and then screening a recombinant antibody bank or single domain antibody bank, produced from lymphoid cells of the animal (for example, using heavy and/or light chain), using the antigen in vitro. According to another approach, the antigen may act on the antibody repertoire by immunizing an animal with the antigen in vivo and then subjecting a recombinant antibody bank or single domain antibody bank, produced from lymphoid cells of the animal, to an affinity maturation. According to another approach, the antigen may act on the antibody repertoire by immunizing an animal with the antigen in vivo and then selecting individual antibody-producing cells which secrete an antibody of interest, and from these selected cells harvesting cDNAs for the variable region of the heavy and light chains (using PCR, for example) and expressing the variable regions of the heavy and light chains in vitro in mammal host cells (referred to as the selected lymphocyte antibody method (SLAM)), thus allowing the selected antibody gene sequences to be further selected and manipulated. In addition, monoclonal antibodies may be selected by expression cloning by expressing the antibody genes for the heavy and light chains in mammal cells and selecting the mammal cells which secrete an antibody having the desired binding affinity.

According to the present invention, defined antigens in the form of RGM-binding domains or polypeptides are provided for screening and counterscreening. Thus, according to the invention polyclonal and monoclonal antibodies may be selected which have a desired characteristics profile according to the invention as defined above.

Various types of antibodies may be produced using the methods according to the invention. These include essentially human antibody, chimeric antibody, humanized antibody, and CDR graft antibody as well as antigen-binding portions thereof.

Methods for producing antibodies according to the invention are described in particular below. A distinction is made between in vivo approaches, in vitro approaches, or a combination of both.

In Vivo Approaches:

Starting with the cells which produce antibodies created in vivo, monoclonal antibodies may be produced using standardized techniques, such as the hybridoma technique originally described by Köhler and Milstein (1975, Nature 256: 495-497) (also see Brown et al. (1981) J. Immunol 127: 539-46; Brown et al. (1980) J. Biol. Chem. 255: 4980-83; Yeh et al. (1976) PNAS 76: 2927-31; and Yeh et al. (1982) Int. J. Cancer 29: 269-75). The technology for production of monoclonal antibody hybridomas is well known (see in general R. H. Kenneth, in Monoclonal Antibodies: A New Dimension In Biological Analyses, Plenum Publishing Corp., New York, N.Y. (1980); E. A. Lerner (1981) Yale J. Biol. Med., 54: 387-402; M. L. Gefter et al. (1977) Somatic Cell Genet., 3: 231-36). For this purpose, an immortalized cell line (typically a myeloma) is fused with lymphocytes (typically splenocytes, lymph node cells, or peripheral blood lymphocytes) of a mammal that has been immunized with the RGM protein according to the invention or derivative/equivalent thereof, and the culture supernatants of the resulting hybridoma cells are screened in order to identify a hybridoma which produces a monoclonal antibody with specificity for RGM protein according to the invention or for a derivative/equivalent thereof. To this end, any given protocol out of many known protocols may be used for the fusion of lymphocytes and immortalized cell lines (also see G. Galfre et al. (1977) Nature 266: 550-52; Gefter et al. Somatic Cell Genet., cited supra; Lerner, Yale J. Biol. Med., cited supra; and Kenneth, Monoclonal Antibodies, cited supra). In addition, one skilled in the art is aware of numerous variations of such methods which likewise may be used. The immortalized cell lines (for example, a myeloma cell line) typically originated from the same mammal species as did the lymphocytes. For example, murine hybridomas may be established by fusing lymphocytes from a mouse immunized with immunogenic preparation according to the invention with an immortalized mouse cell line. Preferred immortalized cell lines are mouse myeloma cell lines which are sensitive for culture medium (HAT medium) containing hypoxanthin, aminopterin, and thymidine. Any given myeloma cell line out of many may be used in a standard manner as fusion partner, for example P3-NS1/1-Ag4-1, P3-x63-Ag8.653, or Sp2/O—Ag14 myeloma line. These myeloma cell lines are available from the American Type Culture Collection (ATCC), Rockville, Md. Typically, HAT-sensitive mouse myeloma cells are fused with mouse splenocytes, using polyethylene glycol (PEG). The hybridoma cells resulting from the fusion are then selected, using HAT medium, thereby killing nonfused and nonproductively fused myeloma cells (nonfused splenocytes die after several days because they are not transformed). Monoclonal antibody-producing hybridoma cells which specifically recognize an RGM protein according to the invention or a derivative/equivalent are identified by screening the hybridoma culture supernatants on such antibodies, for example by using a standard ELISA assay to select antibodies which are able to specifically bind RGM protein according to the invention or a derivative/equivalent thereof.

Depending on the type of antibody desired, various host animals are used for the in vivo immunization. A host which expresses an endogenous version of the antigen of interest itself may be used. Alternatively, a host may be used which has been made deficient for an endogenous version of the antigen of interest. It has been shown, for example, that mice that have been made deficient by homologous recombination at the corresponding endogenous gene for a given endogenous protein (i.e., knockout mice) produce a humoral response to the protein with which they have been immunized, and therefore may be used to produce high-affinity monoclonal antibody against the protein (see, for example, Roes, J. et al. (1995) J. Immunol. Methods 183: 231-237; Lunn, M. P. et al. (2000) J. Neurochem. 75: 404-412).

For the production of nonhuman antibody directed against RGM protein according to the invention or a derivative/equivalent thereof, many nonhuman mammals are suitable as hosts for the antibody production. These include mice, rats, chickens, camelids, rabbits, and goats (and knockout versions thereof), although mice are preferred for hybridoma production. Furthermore, for production of essentially human antibody against a human antigen with dual specificity a nonhuman host animal may be used which expresses a human antibody repertoire. Such nonhuman animals include transgenic animals (mice, for example) which carry the human immunoglobulin transgene (chimeric hu-PBMC-SCID mice), and human/mouse radiation chimera, described in greater detail below.

According to one embodiment, the animal which is immunized with an RGM protein according to the invention or derivative/equivalent thereof is a nonhuman mammal, preferably a mouse, which has been made transgenic using human immunoglobulin genes, so that the nonhuman mammal produces human antibodies following an antigenic stimulus. Typically, immunoglobulin transgenes for heavy and light chains with a human germline configuration are inserted in such animals, the animals having been modified in such a way that their endogenous loci for heavy and light chain are inactive. If such animals are stimulated with antigen (for example, with a human antigen), antibodies are produced which originate from the human immunoglobulin sequences (i.e., human antibody). Human monoclonal antibody may be produced from the lymphocytes of such animals, using standardized hybridoma technology. For a further description of mice made transgenic using human immunoglobulins and their use in the production of human antibody, see, for example U.S. Pat. No. 5,939,598, WO 96/33735, WO 96/34096, WO 98/24893, and WO 99/53049 (Abgenix Inc.), and U.S. Pat. No. 5,545,806, U.S. Pat. No. 5,569,825, U.S. Pat. No. 5,625,126, U.S. Pat. No. 5,633,425, U.S. Pat. No. 5,661,016, U.S. Pat. No. 5,770,429, U.S. Pat. No. 5,814,318, U.S. Pat. No. 5,877,397, and WO 99/45962 (Genpharm Inc.); also see MacQuitty, J. J. and Kay, R. M. (1992) Science 257: 1188; Taylor, L. D. et al. (1992) Nucleic Acids Res. 20: 6287-6295; Lonberg, N. et al. (1994) Nature 368: 856-859; Lonberg, N. and Huszar, D. (1995) Int. Rev. Immunol. 13: 65-93; Harding, F. A. and Lonberg, N. (1995) Ann. N.Y. Acad. Sci. 764: 536-546; Fishwild, D. M. et al. (1996) Nature Biotechnology 14: 845-851; Mendez, M. J. et al. (1997) Nature Genetics 15: 146-156; Green, L. L. and Jakobovits, A. (1998) J. Exp. Med. 188: 483-495; Green, L. L. (1999) J. Immunol. Methods 231: 11-23; Yang, X. D. et al. (1999) J. Leukoc. Biol. 66: 401-410; and Gallo, M. L. et al. (2000) Eur. J. Immunol. 30: 534-540.

According to a further embodiment, the animal which is immunized with RGM protein according to the invention or a derivative/equivalent thereof may be a mouse with severe combined immunodeficiency (SCID) which has been reconstituted with human peripheral mononuclear blood cells or lymphoid cells, or precursors thereof. Such mice, referred to as chimeric hu-PBMC-SCID mice, produce demonstrated human immunoglobulin responses following an antigenic stimulus. For a further description of these mice and their use for antibody production, see, for example, Leader, K. A. et al. (1992) Immunology 76: 229-234; Bombil, F. et al. (1996) Immunobiol. 195: 360-375; Murphy, W. J. et al. (1996) Semin. Immunol. 8: 233-241; Herz, U. et al. (1997) Int. Arch. Allergy Immunol. 113: 150-152; Albert, S. E. et al. (1997) J. Immunol. 159: 1393-1403; Nguyen, H. et al. (1997) Microbiol. Immunol. 41: 901-907; Arai, K. et al. (1998) J. Immunol. Methods 217: 79-85; Yoshinari, K. and Arai, K. (1998) Hybridoma 17: 41-45; Hutchins, W. A. et al. (1999) Hybridoma 18: 121-129; Murphy, W. J. et al. (1999) Clin. Immunol. 90: 22-27; Smithson, S. L. et al. (1999) Mol. Immunol. 36: 113-124; Chamat, S. et al. (1999) J. Infect. Diseases 180: 268-277; and Heard, C. et al. (1999) Molec. Med. 5: 35-45.

According to a further embodiment, the animal which is immunized with RGM protein according to the invention or a derivative/equivalent thereof is a mouse which has been administered a lethal dose of total-body radiation, then protected against radiation using bone marrow cells from mice with severe combined immunodeficiency (SCID), and then receiving a transplant of functional human lymphocytes. This chimeric type, referred to as a Trimera system, is used to produce human monoclonal antibodies by immunizing the mice with the antigen of interest and then producing monoclonal antibodies using standardized hybridoma technology. For a further description of these mice and their use for antibody production, see, for example, Eren, R. et al. (1998) Immunology 93: 154-161; Reisner, Y. and Dagan, S. (1998) Trends Biotechnol. 16: 242-246; Ilan, E. et al. (1999) Hepatology 29: 553-562; and Bocher, W. O. et al. (1999) Immunology 96: 634-641.

In Vitro Approaches:

As an alternative to the production of antibodies according to the invention by immunization and selection, antibodies according to the invention may be identified and isolated by screening a recombinant combinatorial immunoglobulin bank with an RGM protein according to the invention or derivative/equivalent thereof in order to isolate members of the immunoglobulin bank which bind specifically to the RGM protein or derivative/equivalent thereof. Kits for creating and screening display banks are commercially available (for example, the Recombinant Phage Antibody System from Pharmacia, Catalog No. 27-9400-01; and the SurfZAP® phage display kit from Stratagene, Catalog No. 240612). In many embodiments the display bank is an scFv bank or an Fab bank. The phage display technique for screening recombinant antibody banks has been previously described.

Examples of methods and compounds which may be used in a particularly advantageous manner for creating and screening antibody display banks may be found in McCafferty et al. in WO 92/01047, U.S. Pat. No. 5,969,108, and EP 589 877 (describes in particular the display of scFv), Ladner et al. in U.S. Pat. No. 5,223,409, U.S. Pat. No. 5,403,484, U.S. Pat. No. 5,571,698, U.S. Pat. No. 5,837,500, and EP 436 597 (describes pill fusion, for example); Dower et al. in WO 91/17271, U.S. Pat. No. 5,427,908, U.S. Pat. No. 5,580,717, and EP 527 839 (describes in particular the display of Fab); Winter et al. in WO 92/20791 and EP 368 684 (describes in particular the cloning of sequences for variable immunoglobulin domains); Griffiths et al. in U.S. Pat. No. 5,885,793 and EP 589 877 (describes in particular the isolation of human antibodies against human antigens, using recombinant banks); Garrard et al. in WO 92/09690 (describes in particular phage expression techniques); Knappik et al. in WO 97/08320 (describes the HuCal human recombinant antibody bank); Salfeld et al. in WO 97/29131 (describes the production of a recombinant human antibody against a human antigen (human tumor necrosis factor alpha) and in vitro affinity maturation of the recombinant antibody), and Salfeld et al. in U.S. Provisional Application No. 60/126,603 and the patent applications based thereon (likewise describes the production of recombinant human antibody against human antigen (human interleukin-12) and the in vitro affinity maturation of the recombinant antibody).

Further descriptions of screenings of recombinant antibody banks may be found in scientific publications, such as Fuchs et al. (1991) Bio/Technology 9: 1370-1372; Hay et al. (1992) Hum. Antibod. Hybridomas 3: 81-85; Huse et al. (1989) Science 246: 1275-1281; Griffiths et al. (1993) EMBO J 12: 725-734; Hawkins et al. (1992) J Mol. Biol. 226: 889-896; Clarkson et al. (1991) Nature 352: 624-628; Gram et al. (1992) PNAS 89: 3576-3580; Garrad et al. (1991) Bio/Technology 9: 1373-1377; Hoogenboom et al. (1991) Nuc. Acid Res. 19: 4133-4137; Barbas et al. (1991) PNAS 88: 7978-7982; McCafferty et al. Nature (1990) 348: 552-554; and Knappik et al. (2000) J. Mol. Biol. 296: 57-86.

As an alternative to the use of bacteriophage display systems, recombinant antibody banks may be expressed on the surface of yeast cells or bacterial cells. Methods for producing and screening banks which are expressed on the surface of yeast cells are described in WO 99/36569. Methods for producing and screening banks which are expressed on the surface of bacterial cells are described in greater detail in WO 98/49286.

As soon as an antibody of interest is identified from a combinatorial bank, the DNAs which code the light and heavy chains of the antibody are isolated using standardized techniques of molecular biology, for example by PCR amplification of DNA from the display packing (for example, the phage) which has been isolated during screening of the bank. One skilled in the art is familiar with nucleotide sequences of genes for light and heavy antibody chains which may be used to produce PCR primers. Many of these types of sequences are described, for example, in Kabat, E. A., et al. (1991) Sequences of Proteins of Immunological Interest, Fifth Edition, U.S. Department of Health and Human Services, NIH Publication No. 91-3242, and the VBASE database for sequences of the human germline.

An antibody or antibody portion according to the invention may be produced by recombinantly expressing genes for light and heavy immunoglobulin chains in a host cell. To recombinantly express an antibody, a host cell is transfected with one or more recombinant expression vectors which carry DNA fragments that code the light and heavy immunoglobulin chains of the antibody, so that the light and heavy chains in the host cell are expressed and preferably secreted into the medium in which the host cells are cultivated. The antibodies may be recovered from this medium. A standardized recombinant DNA methodology is used to obtain genes for heavy and light antibody chains, insert these genes into recombinant expression vectors, and introduce the vectors into host cells. Such a methodology is described, for example, in Sambrook, Fritsch and Maniatis (Eds.), Molecular Cloning: A Laboratory Manual, Second Edition, Cold Spring Harbor, N.Y., (1989); Ausubel, F. M. et al. (Eds.), Current Protocols in Molecular Biology, Greene Publishing Associates, (1989); and in U.S. Pat. No. 4,816,397 by Boss et al.

As soon as DNA fragments which code the VH and VL segments of the antibody of interest are obtained, these DNA fragments may be further manipulated using standardized recombinant DNA techniques, for example to convert the genes for variable regions into genes for full-length antibody chains, genes for Fab fragments, or an scFv gene. In these manipulations, a VL- or VH-coding DNA fragment is operatively linked with an additional DNA fragment which codes another protein, for example a constant antibody region or a flexible linker. In this case the term “operatively linked” means that the two DNA fragments are joined together in such a way that the amino acid sequences coded by the two DNA fragments remain in the reading frame (in-frame).

The isolated DNA which codes the VH region may be converted to a gene for a full-length heavy chain by operatively linking the DNA which codes the VH region with another DNA molecule which codes constant regions of the heavy chain (CH1, CH2, and CH3). The sequences of genes for constant regions of human heavy chains are well known (see, for example, Kabat, E. A., et al. (1991) Sequences of Proteins of Immunological Interest, Fifth Edition, U.S. Department of Health and Human Services, NIH Publication No. 91-3242), and DNA fragments which span these regions may be obtained using standardized PCR amplification. The constant region of the heavy chain may be a constant region composed of IgG1, IgG2, IgG3, IgG4, IgA, IgE, IgM, or IgD, a constant region composed of IgG1 or IgG4 being preferred. To obtain a gene for an Fab fragment of the heavy chain, the VH-coding DNA may be operatively linked with an additional DNA molecule which codes only the constant region CH1 of the heavy chain.

The isolated DNA which codes the VL region may be converted to a gene for a full-length light chain (as well as a gene for an Fab light chain) by operatively linking the VL-coding DNA with an additional DNA molecule which codes the constant region CL of the light chain. The sequences of genes of the constant region of the human light chain are well known (see Kabat, E. A., et al. (1991) Sequences of Proteins of Immunological Interest, Fifth Edition, U.S. Department of Health and Human Services, NIH Publication No. 91-3242), and DNA fragments which span these regions may be obtained using standardized PCR amplification. The constant region of the light chain may be a constant kappa or lambda region, a constant kappa region being preferred.

To produce an scFv gene, the VH- and VL-coding DNA fragments may be operatively linked with an additional coding fragment which codes a flexible linker, for example the amino acid sequence (Gly₄-Ser)₃, so that the VH and VL sequences are expressed as continuous single-chain protein, the VL and VH regions being joined together via a flexible linker (see Bird et al. (1988) Science 242: 423-426; Huston et al. (1988) Proc. Natl. Acad. Sci. USA 85: 5879-5883; and McCafferty et al., Nature (1990) 348: 552-554).

VH and VL single domains with specificity for RGM protein according to the invention or a derivative/equivalent thereof may be isolated from single-domain banks by using the methods described above. Two VH single-domain chains (with or without CH1), or two VL chains, or a pair composed of one VH chain and one VL chain having the desired specificity may be used to bind RGM proteins according to the invention or derivatives/equivalents thereof.

To express the recombinant antibody or antibody portions according to the invention, the DNAs which code the light and heavy chains of partial or full length may be inserted into expression vectors, thereby operatively linking the genes with transcriptional and translational control sequences. In this context, the term “operatively linked” means that an antibody gene is ligated in a vector in such a way that transcriptional and translational control sequences within the vector fulfill their intended function for regulating the transcription and translation of the antibody gene.

The expression vector and the expression control sequences are selected so that they are compatible with the host cell used for the expression. The gene for the light antibody chain and the gene for the heavy antibody chain may be inserted into different vectors, or both genes may be inserted into the same expression vector, which is generally the case. The antibody genes are inserted into the expression vector using standardized methods (for example, ligation of complementary restriction interfaces at the antibody gene fragment and vector, or ligation of blunt ends if no restriction interfaces are present). The expression vector may carry sequences for constant antibody regions before the sequences for the light and heavy chains are inserted. For example, in one approach the VH and VL sequences are converted to full-length antibody genes by inserting the sequences into expression vectors which already code the constant regions for heavy and light chains, thereby operatively linking the VH segment with the CH segment(s) within the vector, and also operatively linking the VL segment with the CL segment within the vector. Additionally or alternatively, the recombinant expression vector may code a signal peptide which simplifies the secretion of the antibody chain from the host cell. The gene for the antibody chain may be cloned into the vector, thereby linking the signal peptide in the reading frame to the N terminus of the gene for the antibody chain. The signal peptide may be an immunoglobulin signal peptide or a heterologous signal peptide (i.e., a signal peptide from a non-immunoglobulin protein). In addition to the genes for the antibody chain, the expression vectors according to the invention may have regulatory sequences which control the expression of the genes for the antibody chain in a host cell. The term “regulatory sequence” encompasses promoters, enhancers, and other expression control elements (polyadenylation signals, for example) which control the transcription or translation of the genes for the antibody chain. Such regulatory sequences are described, for example, in Goeddel; Gene Expression Technology: Methods in Enzymology 185, Academic Press, San Diego, Calif. (1990). It is known to one skilled in the art that the design of the expression vector, which includes the selection of regulatory sequences, may depend on factors such as the selection of the host cell to be transformed, the desired expression intensity of the protein, etc. Preferred regulatory sequences for expression in mammal host cells include viral elements which result in intense protein expression in mammal cells, such as promoters and/or enhancers which originate from cytomegalovirus (CMV) (such as the CMV promoter/enhancer), simian virus 40 (SV40) (such as the SV40 promoter/enhancer), adenovirus (for example, the adenovirus major late promoter (AdMLP), and polyoma. For a further description of viral regulatory elements and sequences thereof, see, for example, U.S. Pat. No. 5,168,062 by Stinski, U.S. Pat. No. 4,510,245 by Bell et al., and U.S. Pat. No. 4,968,615 by Schaffner et al.

In addition to the genes for the antibody chain and the regulatory sequences, recombinant expression vectors according to the invention may contain additional sequences, such as sequences which regulate the replication of the vector in host cells (replication start points, for example) and selectable marker genes. The selectable marker genes simplify the selection of host cells in which the vector has been introduced (see, for example, U.S. Pat. No. 4,399,216, U.S. Pat. No. 4,634,665, and U.S. Pat. No. 5,179,017, all by Axel et al.). For example, it is common for the selectable marker gene to make a host cell, in which the vector has been introduced, resistant to active substances such as G418, hygromycin, or methotrexate. Preferred selectable marker genes include the gene for dihydrofolate reductase (DHFR) (for use in dhfr⁻ host cells with methotrexate selection/amplification) and the neogene (for G418 selection).

For the expression of the light and heavy chains, the expression vector(s) which code the heavy and light chains is/are transfected into a host cell, using standardized techniques. The various forms of the “transfection” encompass a number of techniques which are usually used to introduce exogenous DNA into a prokaryotic or eukaryotic host cell, for example electroporation, calcium phosphate precipitation, DEAE dextran transfection, and the like. Although it is theoretically possible to express the antibody according to the invention in either prokaryotic or eukaryotic host cells, the expression of the antibody in eukaryotic cells and in particular in mammal host cells is preferred, since the probability that a correctly folded and immunologically active antibody is combined and secreted in such eukaryotic cells and in particular mammal cells is greater than in prokaryotic cells. With regard to the prokaryotic expression of antibody genes, it has been reported that such are ineffective for the production of high yields of active antibody (Boss, M. A. and Wood, C. R. (1985) Immunology Today 6: 12-13).

Mammal host cells which are preferred for the expression of recombinant antibody according to the invention include CHO cells (including dhfr⁻ CHO cells, described in Urlaub and Chasin, (1980) Proc. Natl. Acad. Sci. USA 77: 4216-4220 and used with a DHFR-selectable marker, described, for example, in R. J. Kaufman and P. A. Sharp (1982) Mol. Biol. 159: 601-621), NS0 myeloma cells, COS cells, and SP2 cells. When recombinant expression vectors which code the antibody genes are introduced to mammal host cells, the antibody is produced by cultivating the host cells until the antibody is expressed in the host cells or, preferably, the antibody is secreted into the culture medium in which the host cells grow. The antibodies may be recovered from the culture medium by using standardized methods for purifying proteins.

Host cells may also be used to produce portions of intact antibodies, such as Fab fragments or scFv molecules. Of course, variations of the previously described procedure are encompassed by the invention. For example, it may be desirable to transfect a host cell with DNA which codes either the light chain or the heavy chain (but not both) of an antibody according to the invention. If light or heavy chains are present which are not necessary for the binding of the antigen of interest, the DNA, which codes either such a light chain or such a heavy chain, or both, may be partially or completely removed using recombinant DNA technology. Molecules which are expressed by such truncated DNA molecules are likewise included in the antibodies according to the invention. Furthermore, bifunctional antibodies may be produced in which one heavy chain and one light chain constitute an antibody according to the invention, and the other heavy chain and light chain have specificity for an antigen other than the one of interest, by crosslinking an antibody according to the invention with a second antibody using standardized chemical methods.

In one preferred system for recombinant expression of an antibody antigen-binding portion thereof according to the invention, a recombinant expression vector which codes both the heavy antibody chain and the light antibody chain is introduced by calcium phosphate-mediated transfection in dhfr⁻ CHO cells. Within the recombinant expression vector the genes for the heavy and light antibody chains are each operatively linked with regulatory CMV enhancers/AdMLP promoter elements to achieve strong transcription of the genes. The recombinant expression vector also carries a DHFR gene by means of which CHO cells which are transfected with the vector may be selected by using methotrexate selection/amplification. The selected transformed host cells are cultivated so that the heavy and light antibody chains are expressed, and intact antibody is recovered from the culture. Standardized techniques of molecular biology are used to produce the recombinant expression vector, transfect the host cells, select the transformants, cultivate the host cells, and recover the antibody from the culture medium. Thus, the invention relates to a method for synthesizing a recombinant antibody according to the invention by cultivating a host cell according to the invention in a suitable culture medium, until a recombinant antibody according to the invention is synthesized. The method may also include isolation of the recombinant antibody from the culture medium.

As an alternative to screening recombinant antibody banks by phage display, other methods known to one skilled in the art may be used to screen large combinatorial banks in order to identify the antibody according to the invention. In one type of an alternative expression system, the recombinant antibody bank is expressed in the form of RNA protein fusions, as described in WO 98/31700 by Szostak and Roberts, and in Roberts, R. W. and Szostak, J. W. (1997) Proc. Natl. Acad. Sci. USA 94: 12297-12302. In this system a covalent fusion is produced by in vitro translation of synthetic mRNAs which on their 3′ end bear a peptidyl acceptor antibiotic puromycin between an mRNA and the peptide or protein which it codes. Thus, a specific mRNA from a complex mixture of mRNAs (for example, a combinatorial bank) may be enriched on the basis of the characteristics of the coded peptides or proteins (for example, of the antibody or a portion thereof), such as the binding of the antibody or a portion thereof to RGM protein according to the invention or a derivative/equivalent thereof. Nucleic acid sequences which code antibodies or portions thereof and which are recovered from the screening of such banks may be expressed using recombinant means in the described manner (for example, in mammal host cells), and may also be subjected to further affinity maturation, either by screening mRNA peptide fusions in further passes in which mutations are inserted into the sequence(s) originally selected, or by using other methods for the in vitro affinity maturation of recombinant antibodies in the above-described manner.

Combinations of in vivo and in vitro approaches:

The antibodies according to the invention may also be produced by using a combination of in vivo and in vitro approaches, such as methods in which RGM protein according to the invention or a derivative/equivalent thereof is first allowed to act on an antibody repertoire in vivo in a host animal in order to stimulate the production of RGM protein or derivative/equivalent-binding antibodies, and then the further antibody selection and/or antibody maturation (i.e., optimization) is performed using one or more in vitro techniques. According to one embodiment, such a combined method may consist in first immunizing a nonhuman animal (for example, a mouse, rat, rabbit, chicken, camelid, goat, or a transgenic version thereof, or a chimeric mouse) with the RGM protein according to the invention or derivative/equivalent thereof in order to stimulate an antibody response to the antigen, and then, using immunoglobulin sequences from lymphocytes which have been stimulated in vivo by the action of the RGM protein or derivative/equivalents [thereof], creating and screening a phage display antibody bank. The first step of this combined procedure may be carried out in the manner described above for the in vivo approaches, while the second step of this procedure may be carried out in the manner described above for the in vitro approaches. Preferred methods for hyperimmunization of nonhuman animals with subsequent in vitro screening of phage display banks which have been created from the stimulated lymphocytes include those described by BioSite Inc.; see, for example, WO 98/47343, WO 91/17271, U.S. Pat. No. 5,427,908, and U.S. Pat. No. 5,580,717.

According to a further embodiment, a combined method consists in first immunizing a nonhuman animal (for example, a mouse, rat, rabbit, chicken, camelid, goat, or a knockout and/or transgenic version thereof, or a chimeric mouse) with an RGM protein according to the invention or derivative/equivalent thereof in order to stimulate an antibody response to the RGM protein or derivative/equivalent thereof, and selecting the lymphocytes which produce the antibody with the desired specificity by screening hybridomas (for example, produced from the immunized animals). The genes for the antibodies or single-domain antibodies are isolated from the selected clone (using standardized cloning methods such as the reverse transcriptase polymerase chain reaction) and subjected to in vitro affinity maturation in order to improve the binding characteristics of the selected antibody or antibodies. The first step of this procedure may be carried out in the manner described above for in vivo approaches, while the second step of this procedure may be carried out in the manner described above for the in vitro approaches, in particular by using methods for in vitro affinity maturation as described in WO 97/29131 and WO 00/56772.

In a further combined method, the recombinant antibody is produced from individual lymphocytes by using a procedure known to one skilled in the art as the selected lymphocyte antibody method (SLAM) and described in U.S. Pat. No. 5,627,052, WO 92/02551, and Babcock, J. S. et al. (1996) Proc. Natl. Acad. Sci. USA 93: 7843-7848. In this method, a nonhuman animal (for example, a mouse, rat, rabbit, chicken, camelid, goat, or a transgenic version thereof, or a chimeric mouse) is first immunized in vivo with RGM protein according to the invention or a derivative/equivalent thereof in order to stimulate an immune response to the RGM protein or derivative/equivalent thereof, and then individual antibody-secreting cells of interest are selected by using an antigen-specific hemolytic plaque assay. To this end, the RGM protein or derivative/equivalent thereof or structurally related molecules of interest may be coupled to sheep erythrocytes, using a linker such as biotin, thereby allowing identification of individual cells which secrete the antibody with suitable specificity, using the hemolytic plaque assay. Following the identification of cells which secrete the antibodies of interest, cDNAs for the variable regions of the light and heavy chains are recovered from the cells by reverse transcriptase PCR, and these variable regions may then be expressed in conjunction with suitable constant immunoglobulin regions (for example, human constant regions) in mammal host cells such as COS or CHO cells. The host cells transfected with amplified immunoglobulin originating from lymphocytes which select in vivo may then be subjected to further in vitro analysis and selection, for example by propagating the transfected cells to isolate cells which express the antibodies with the desired specificity. The amplified immunoglobulin sequences may be further manipulated in vitro.

6. Pharmaceutical Agents

6.1 General Information

The subject matter of the present invention also concerns pharmaceutical agents (compositions) which contain as active substance protein according to the invention (RGM protein; RGM protein-binding ligands, such as anti-RGM protein-antibodies) or a coding RGM protein nucleic acid sequence and optionally a pharmaceutically acceptable carrier. Pharmaceutical compositions according to the invention may also contain at least one additional therapeutic agent, for example one or more additional therapeutic agents for treating one of the diseases described herein.

Pharmaceutically acceptable carriers include all solvents, dispersion media, coatings, antimicrobial agents, isotonisizing and absorption-delaying agents, and the like, provided that these are physiologically compatible.

Pharmaceutically acceptable carriers include, for example, water, saline solution, phosphate-buffered saline solution, lactose, dextrose, sucrose, sorbitol, mannitol, starches, gum arabic, calcium phosphate, alginates, gum tragacanth, gelatins, calcium silicate, microcrystalline cellulose, polyvinylpyrrolidone, cellulose, water, syrup, and methylcellulose. The formulations may also include pharmaceutically acceptable carriers or common adjuvants such as lubricants, for example talc, magnesium stearate, and mineral oil; wetting agents; emulsifying and suspending agents; preservatives, such as methyl- and propylhydroxy benzoates; antioxidants; anti-irritants; chelate-forming agents; coating agents; emulsion stabilizers; film-forming agents; gel-forming agents; odor-masking agents; flavorants; resins; hydrocolloids; solvents; solubilizers; neutralizing agents; permeation accelerators; pigments; quaternary ammonium compounds; moisturizers and emollients; salve, cream, or oil bases; silicone derivatives; spreading agents; stabilizers; sterilants; suppository bases; tableting adjuvants, such as binders, fillers, lubricants, disintegrants, or coatings; propellants; drying agents; opacifiers; thickeners; waxes; softeners; and white oils. Designs in this regard are based on knowledge of one skilled in the art, as described, for example, in Fiedler, H. P., Lexikon der Hilfsstoffe für Pharmazie, Kosmetik and angrenzende Gebiete [Lexicon of Adjuvants for Pharmaceutical, Cosmetic, and Related Areas], 4th Ed., Aulendorf: ECV-Editio-Kantor-Verlag, 1996. Also see Hager's Handbuch der Pharmazeutischen Praxis [Hager's Handbook of Pharmaceutical Practice], Springer Verlag, Heidelberg.

The pharmaceutical compositions may be suitable for parenteral administration, for example. For this purpose the active substance, the antibody, for example, is preferably prepared in the form of injectable solutions with an active substance content of 0.1-250 mg/mL. The injectable solutions may be provided in liquid or lyophilized form in flint glass or a vial, an ampule, or a filled syringe as dosage form.

The buffer may contain L-histidine (1-50 mM, preferably 5-10 mM) and may have a pH of 5.0-7.0, preferably 6.0. Without being limited thereto, further suitable buffers include sodium succinate, sodium citrate, sodium phosphate, or potassium phosphate buffer.

Sodium chloride may be used to adjust the tonicity of the solution to a concentration of 0-300 mM (preferably 150 mM for a liquid dosage form). Cryogenic protective agents such as sucrose (for example, 0-10%, preferably 0.5-1.0% (w/w)) may be incorporated for a lyophilized dosage form. Other suitable cryogenic protective agents include trehalose and lactose. Fillers such as mannitol (for example, 1-10%, preferably 2-4% (w/w)) may be incorporated for a lyophilized dosage form. Stabilizers such as L-methionine (for example, 51-50 mM, preferably 5-10 mM) may also be used in liquid as well as lyophilized dosage forms. Other suitable fillers include glycine and arginine. Surfactants may also be used, for example polysorbate 80 (for example, 0-0.05%, preferably 0.005-0.01% (w/w)). Other surfactants include polysorbate 20 and Brij surfactants.

The compositions according to the invention may assume a number of forms. These include liquid, semisolid, and solid dosage forms, such as liquid solutions (for example, injectable and infusable solutions, lotions, eyedrops and eardrops), liposomes, dispersions, or suspensions, and solid forms such as meals, powders, granulates, tablets, pastilles, sachets, cachets, dragees, capsules such as hard and soft gelatin capsules, suppositories or vaginal administration forms, or semisolid administration forms such as salves, creams, hydrogels, pastes, or plasters. Implanted dispensers may also be used for administering active substances according to the invention. The preferred form depends on the intended type of administration and the therapeutic application. Compositions in the form of injectable or infusable solutions are usually preferred. An example of one suitable administration path is parenteral (for example, intravenous, subcutaneous, intraperitoneal, intramuscular). According to one preferred embodiment the active substance is administered by intravenous infusion or injection. According to a further preferred embodiment, the active substance is administered by intramuscular or subcutaneous injection.

Therapeutic compositions must typically be sterile and be stable under manufacturing and storage conditions. The compositions may be formulated in the form of a solution, microemulsion, dispersion, liposomal structure, or other ordered structure suitable for high active substance concentrations. Sterile injectable solutions may be produced by introducing the active compound (such as the antibody, for example) in the necessary quantity into a suitable solvent, optionally with one or a combination of the ingredients listed above, and then performing sterile filtration. Dispersions are generally prepared by introducing the active compound into a sterile vehicle which contains a base dispersion medium and optionally other necessary ingredients. When a sterile lyophilized powder is used to produce sterile injectable solutions, vacuum drying and spray drying represent preferred manufacturing methods for obtaining a powder of the active ingredient and optionally other desired ingredients from a solution that has previously been sterilely filtered. The correct flow characteristics of a solution may be maintained by using a coating such as lecithin, and for dispersions the required particle size is maintained or surfactants are used. Extended absorption of injectable compositions may be achieved by introducing an agent which delays the absorption, for example monostearate salts and gelatins, into the composition.

The active substances according to the invention may be administered using a number of methods known to one skilled in the art, although for many therapeutic applications subcutaneous injection, intravenous injection, or infusion represent the preferred type of administration. One skilled in the art is aware that the path and/or type of administration depend on the desired result. According to certain embodiments the active compound may be prepared using a carrier which protects the compound from rapid release, such as a controlled-release formulation, for example, which includes implants, transdermal plasters, and microencapsulated delivery systems. Biodegradable, biocompatible polymers may be used, such as ethylene vinyl acetate, polyanhydrides, polyglycolic acid, collagen, polyorthoesters, and polylactic acid. The methods for preparing such formulations are generally known to one skilled in the art; see, for example, Sustained and Controlled Release Drug Delivery Systems, J. R. Robinson, Ed., Marcel Dekker, Inc., New York, 1978.

According to certain embodiments, an active substance according to the invention may be administered orally, for example in an inert diluent or an assimilable edible carrier. The active substance (and other ingredients, if desired) may also be encapsulated in a hard or soft gelatin capsule, pressed into tablets, or added directly to food. For oral therapeutic administration, the active substances may be mixed with excipients and used in the form of chewable tablets, buccal tablets, capsules, elixirs, suspensions, syrups, and the like. If an active substance according to the invention is to be administered via a path other than parenteral, it may be necessary to select a coating from a material which prevents inactivation of the active substance.

The active substances according to the invention may be administered together with one or more additional therapeutic agents which are suitable in the treatment of the diseases described above.

The pharmaceutical compositions of the present invention generally contain a therapeutically effective quantity or a prophylactically effective quantity of at least one active substance according to the invention. Dosage plans may be selected and adapted, depending on the desired treatment, or whether a therapeutic or prophylactic treatment is desired, for example. For example, a single dose, several separate doses distributed over time, or an increasing or decreasing dose may be administered, depending on the requirements of the therapeutic situation. It is advantageous in particular to formulate parenteral compositions in single-dosage form in order to simplify administration and ensure uniformity of the dosages.

The attending physician can easily specify the administration form, type of administration, and dosage which are most suitable for the particular treatment and the particular active substance.

A therapeutically or prophylactically effective quantity of an active substance according to the invention may, for example, be in the range of 0.1-20 mg/kg, preferably 1-10 mg/kg, without being limited thereto. Of course, these quantities may vary, depending on the nature and severity of the condition to be alleviated.

6.2 Vaccines

The RGM proteins according to the invention and derivatives/equivalents thereof may be used as immunogen for vaccination of a patient to be treated.

For this purpose, suitable vaccines are generally a pharmaceutical composition which contains at least one RGM protein according to the invention and/or at least one derivative/equivalent thereof according to the invention. The composition may also contain a physiologically acceptable carrier and optionally further adjuvants, for example immune stimulants.

Although in principle suitable carriers may be selected as desired, the type of carrier generally depends on the administration path. Thus, the vaccines according to the invention may in particular be formulated in a form that is suitable for parenteral, for example intravenous, intramuscular, and subcutaneous, administration. In these cases the carrier preferably contains water, saline solution, alcohol, a fat, a wax, and/or a buffer.

Any given number of immune stimulants may be used in the vaccines according to the invention. For example, an adjuvant may be incorporated. Most adjuvants contain a substance, such as aluminum hydroxide or a mineral oil, as well as a protein derived from lipid A, Bordetella pertussis, or Mycobacterium tuberculosis, which is designed to protect the antigen from rapid destruction. Suitable adjuvants are generally commercially available, for example complete or incomplete Freund's adjuvant; AS-2; aluminum salts, such as aluminum hydroxide (optionally in the form of a gel) or aluminum phosphate; calcium, iron, or zinc salts; an insoluble suspension of acylated tyrosine; acylated sugars; cationically or anionically derivatized polysaccharides; polyphosphazenes; biodegradable microspheres; or monophosphoryl lipid A. Cytokines such as GM-CSF or interleukin-2, -7, or -12 may also be used as adjuvants.

7. Treatment Methods

7.1. Treatment of Neuronal Diseases

It is known from the prior art that in injuries to the central nervous system an accumulation of RGM protein is observed at the lesion site (see Schwab et al., loc. cit.). At the same time, this prevents new growth of the nerve fibers. Neutralization of RGM A in the spinal cord injury model in rats by use of a polyclonal RGM A-specific antibody resulted in regeneration and functional recovery (Hata K. et al., J. Cell Biol. 173: 47-58, 2006). The damaging or inhibitory effect on the growth of nerve fiber is mediated by the binding of RGM A to the receptor molecule neogenin (Conrad S. et al., J. Biol. Chem. 282: 16423-16433, 2007). Modulation, in particular inhibition, of the interaction between RGM and the receptor molecule neogenin would therefore be suitable to arrest the inhibitory activity of RGM on the growth of nerve fiber.

7.2. Treatment of Tumor Diseases

Evidence has been available for quite some time which implicates neogenin in the etiology and/or progression of tumor diseases. For example, Meyerhardt et al. reported in Oncogene (1997) 14, 1129-1136 that neogenin was detectable in more than fifty different cancer cell lines, including glioblastoma, medulloblastoma, and neuroblastoma cell lines, as well as cell lines of colorectal, breast, pancreatic, and cervical cancers. Overexpression of neogenin has also been observed in cancer of the esophagus (Hue et al., Clinical Cancer Research (2001) 7, 2213-2221). A recent systematic analysis of the expression profiles of 3588 genes in 211 pulmonary adenocarcinoma patients provided additional information concerning the involvement of neogenin in the etiology and progression of tumor disease (Berrar et al., J. Comput. Biol. (2005) 12 (5), 534-544).

Since it is also known that RGM has a potential tumor-promoting effect in that it is able to prevent cell death by binding to the neogenin receptor associated with the tumor cell (Matsunaga et al., Nature Cell Biol. 6, 749-755, 2004), a new therapeutic approach for the treatment of tumor diseases could be provided by modulation of the RGM-neogenin interaction, in particular by interruption of interaction using specific anti-RGM antibodies.

On the other hand, fragments of the human RGM A protein which activate neogenin receptors may inhibit tumor cell migration or metastasis of neogenin-positive tumor cells. This inhibition of tumor cell migration could occur in a manner analogous to the inhibition of nerve fiber growth. Nerve fibers also grow in an invasive manner, but, in contrast to tumor cells, generally in a controlled invasive manner. This is supported by the recent identification of hRGM A as a potential tumor suppressor candidate in classic Hodgkin's lymphoma (Feys et al., Haematologica 2007, Vol. 92, 913-20).

7.3. Treatment of Ironmetabolism Diseases

RGM C, also known as hemojuvelin, is of fundamental importance for iron metabolism in the bodies of humans and animals. Juvenile hemochromatosis is an inherited, relatively rare iron metabolism disease which manifests in the form of iron overload in the organism. This disease is caused by mutations in the hemojuvelin molecule (see Huang et al., The Journal of Clinical Investigation (2005), 115, 2087-2091). The administration of functional RGM proteins according to the invention or the active domains thereof therefore represents a feasible therapeutic approach for alleviating such iron metabolism diseases. For anemia in chronic diseases, inflammatory or malignant processes result in massive up-regulation of certain cytokines, such as tumor necrosis factor alpha, for example, (Weiss M. D. and Goodnough, L. T., New Engl. J. Med. 352: 1011-1022, 2005). These cytokines are potent inductors of the most important regulator of iron metabolism, the peptide hormone hepcidin, and overproduction or accumulation of hepcidin is regarded as a significant reason for the pathogenesis of anemia in chronic disease. Recent in vivo data for mice demonstrate that Fc-conjugated RGM C (Fc hemojuvelin) inhibits hepcidin expression and raises the serum iron level (Babitt, J. L. et al., The Journal of Clinical Investigation, 2007, Vol. 117, 7, 1933-1939). The interaction of RGM proteins with BMP proteins is an important factor in this regulation (Babitt, J. L. et al., Nature Genetics, 2006, Vol. 48, 5, 531-539). Fc-conjugated RGM C or Fc-conjugated fragments of RGM C, RGM A, or [RGM]B, which interact with BMP proteins, may therefore be used as therapeutic agents for treatment of anemia in chronic disease.

7.4. Promotion of Bone Tissue Formation

Information is available from the prior art that a member of the RGM family of proteins, namely, RGM B, also known by the name DRAGON, is involved in bone morphogenesis. For example, Samad et al. in JBC Papers 2005, Vol. 280, 14122-14129 describe the interaction between DRAGON and the Type I and Type II receptors of bone morphogenetic protein (BMP). A bone growth-promoting effect, and thus a new therapeutic approach for treatment of bone growth disorders or bone injuries, is therefore conceivable by administration of RGM polypeptides according to the invention. All three RGM proteins (RGM A, B, C) interact with various members of the BMP family and increase the activation of the BMP signal path (Babitt, J. L. et al., Nature Genetics, 2006, Vol. 48, 5, 531-539; Babitt, J. L. et al., J. Biol. Chem., 2005, Vol. 280, 33, 29820-29827; Babitt, J. L. et al., The Journal of Clinical Investigation, 2007, Vol. 117, 7, 1933-1939; Samad, T. A. et al., J. Biol. Chem., 2005, Vol. 280. 14, 14122-14129; Halbrooks et al., J. Molecular Signaling 2, 4: 2007 (published in electronic form).

7.5 Treatment of Autoimmune Diseases

Indications that active substances according to the invention may be feasible for the treatment of autoimmune diseases are found in the following publications: Urist et al., Prog. Clin. Biol. Res. 1985, Vol. 187: 77-96; Lories and Luyten, Cytokine & Growth Factor Reviews 2005, Vol. 16, 287-298.

8. Diagnostic Methods

RGM protein and derivatives/equivalents according to the above definition, as well as antibodies directed against same, are named in particular as diagnostic agents according to the invention.

The present invention therefore allows improved qualitative or quantitative determination of the medical conditions defined above by detection of antigens or antibodies which are typical for the disease.

The determination is preferably carried out using immunological methods. In principle, this may be achieved using any analytical or diagnostic test method in which antibodies are used. These include agglutination and precipitation techniques, immunoassays, immunohistochemical methods, and immunoblot techniques, for example Western blotting or dot blot methods. In vivo methods such as imaging processes are also included.

Use in immunoassays is advantageous. Competitive immunoassays, i.e., antigen and labeled antigen (tracer) competing for the antibody binding, as well as sandwich immunoassays, i.e., binding of specific antibodies to the antigen, are detected using a second antibody which is usually labeled. These assays may be homogeneous, i.e., with no separation into a solid and a liquid phase, as well as heterogeneous, i.e., in which bound labels are separated from nonbound labels, for example using antibodies bound to the solid phase. The various heterogeneous and homogeneous immunoassay formats may be assigned to given classes, depending on the labeling and the measurement methods, for example radioimmunoassays (RIA), enzyme-linked immunosorbent assay (ELISA), fluorescence immunoassay (FIA), luminescence immunoassay (LIA), time resolved FIA (TRFIA), immune activation (IMAC), enzyme multiplied immune test (EMIT), turbodimetric immunoassay (TIA), and immuno-PCR (I-PCR).

Competitive immunoassays are preferred for antigen determination according to the invention. Labeled antigen (tracer) competes with the sample antigen to be quantified for binding to the antibody used. The quantity of antigen in the sample, i.e., the quantity of antigen, may be determined from the quantity of tracer displaced, using a standard curve.

Of the labels available for this purpose, enzymes have proven to be advantageous. For example, systems based on peroxidase, in particular horseradish peroxidase, alkaline phosphatase, and β-D-galactosidase may be used. Specific substrates are available for these enzymes, whose reaction may be tracked photometrically, for example. Suitable substrate systems are based on p-nitrophenyl phosphate (PNPP), 5-bromo-4-chloro-3-indolyl phosphate/nitroblue tetrazolium (BCIP/NBT), Fast Red/naphthol AS-TS phosphate for the alkaline phosphatase; 2,2-azino-bis-(3-ethylbenzothiazoline-6-sulfonic acid) (ABTS), o-phenylenediamine (OPT), 3,3′,5,5′-tetramethylbenzidine (TMB), o-dianisidine, 5-aminosalicylic acid, 3-dimethylaminobenzoic acid (DMAB), and 3-methyl-2-benzothiazoline hydrazone (MBTH) for the peroxidases; o-nitrophenyl-β-D-galactoside (ONPG), p-nitrophenyl-β-D-galactoside, and 4-methylumbelliphenyl-β-D-galactoside (MUG) for the β-D-galactosidase. In many cases these substrate systems are commercially available in ready-to-use form, for example in the form of tablets, which may also contain further reagents such as useful buffers and the like.

Labels may be coupled to peptides or antibodies for producing tracers in a manner known as such. In addition, a number of labels which have been usefully modified for conjugation to proteins are available, for example biotin, avidin, extravidin, or streptavidin-conjugated enzymes, maleimide-activated enzymes, and the like. These labels may be reacted directly with the molecule to be used according to the invention.

If a heterogeneous immunoassay format is selected, for the purpose of separation, for example using an anti-idiotypical antibody coupled to the carrier, such as an antibody directed against rabbit IgG, the antigen-antibody complex may be bound to the substrate. Substrates, in particular microtiter plates, which are coated with the corresponding antibodies are known and commercially available.

A further subject matter of the present invention concerns immunoassay sets containing at least one antibody described above, and additional components. These sets are assemblies, generally in the form of packaged units, of agents for carrying out a determination according to the invention. These agents are preferably provided in essentially ready-to-use form in order to simplify handling as much as possible. In one advantageous layout, the immunoassay is provided in the form of a kit. A kit generally includes several receptacles for separate provision of components. All of the components may be provided in a ready-to-use dilution, as a concentrate for dilution, or as a lyophilizate for dissolution or suspension; individual components or all of same may be frozen, or stored at ambient temperature until used. Sera are preferably quick-frozen, preferably at −20° C., for example, so that in such cases an immunoassay preferably must be kept at freezing temperature before use.

Additional components which may be included with the immunoassay include the following: standard protein, tracer, control serum, microtiter plates, preferably coated with antibody, buffers, for example for testing, washing, or reaction of the substrate, and the enzyme substrate itself.

General principles of immunoassays and the production and use of antibodies as aids in the laboratory and clinic are described, for example, in Antibodies, A Laboratory Manual (Harlow, E., and Lane, D., Eds., Cold Spring Harbor Laboratory, Cold Spring Harbor, N.Y., 1988).

9. Screening Methods

The subject matter of the invention further concerns methods for detection of effectors of the RGM receptor (neogenin and/or BMP), wherein a sample which is suspected of being an effector is incubated with an RGM protein or polypeptide, and the assay is analyzed for the formation of an effector-RGM protein complex.

Such effectors may have an agonistic, partially agonistic, antagonistic, or inverse agonistic effect. These may be synthetic low-molecular substances, synthetic peptides, natural or synthetic antibody molecules, or natural substances.

Such methods according to the invention are generally carried out as in vitro screening processes, by means of which the substances which appear to be most promising for future use may be sorted out from a number of various substances.

For example, by use of combinatorial chemistry extensive substance banks may be created which contain many potential active substances. The sampling of combinatorial substance libraries for substances having desired activity may be automated. Screening robots are used to efficiently evaluate the individual assays, which preferably are provided on microtiter plates. Thus, the present invention also relates to screening methods, i.e., both primary and secondary screening methods, in which preferably at least one of the methods described below is used. If several methods are used, this may be performed at the same or different times for a single sample or for various samples of a substance to be analyzed.

One effective technique for carrying out such methods is the scintillation proximity assay, or

SPA for short, which is known in the field of active substance screening. Kits and components for performing this assay are commercially available, for example from Amersham Pharmacia Biotech. The method operates on the principle of immobilizing solubilized or membrane-bound receptors on small fluoromicrospheres containing scintillation substance. If a radioligand, for example, binds to the immobilized receptors, the scintillation substance is stimulated to emit light, since the spatial proximity of the scintillation substance to the radioligand is specified.

Another effective technique for carrying out such methods is the FlashPlate® technology, which is known in the field of active substance screening. Kits and components for performing this assay are commercially available, for example from NEN Life Science Products. The principle of operation is likewise based on microtiter plates (96-well or 384-well) which are coated with scintillation substance.

The substances or portions of substance mixtures which may be identified using these methods are likewise subject matter of the present invention.

The invention is described in greater detail with reference to the following nonlimiting production and application examples.

### Experimental Section

### 1. General Procedures

SDS Polyacrylamide Gel Electrophoresis

Proteins were separated in SDS polyacrylamide gels according to their molecular weights (4-20% tris-glycine gel: Invitrogen EC6025BOX; 10-20% Tricine gel: Invitrogen #EC6625BOX). The samples were mixed with NuPage SDS sample buffer (4×), using reducing agent. After incubation for 10 minutes in a Thermomixer at 95° C. the samples were developed at 125 V, using tris-glycine or Tricine SDS running buffer (Invitrogen). SeeBlue or SeeBlue Plus 2 (Invitrogen) were used as molecular weight standard proteins. The gels were stained with Coomassie stain or transferred to nitrocellulose (nitrocellulose membrane filter paper sandwich (Invitrogen #LC2001).

Coomassie Staining

For detection of proteins in polyacrylamide gels, the proteins were stained with Coomassie stain after the gel run. The gels were stained for 1 hour in SimplyBlue Safestain stain solution (Invitrogen) on a membrane (0.2-μm pore), or alternatively, in colloidal Coomassie stain (0.25% Coomassie Blue R250/L, 45% methanol, 10% acetic acid). Destaining was performed using deionized water or destaining solution (40% methanol, 10% acetic acid) until protein bands were clearly visible.

Western Blot

Filter paper and nitrocellulose were impregnated with Novex transfer buffer for 10 minutes with 20% methanol. The blotting was performed in a Novex chamber at constant current (100 mA) over a period of 2 hours at room temperature.

Dot Blot

2 μL protein in various concentrations in TTBS buffer was dabbed onto dry nitrocellulose membranes. The following dilutions were used:

Dilutions:

| | | |
|---|---|---|
| a) | 100 μg/mL ≈ | 200 ng/spot |
| b) | 50 μg/mL ≈ | 100 ng/spot |
| c) | 10 μg/mL ≈ | 20 ng/spot |
| d) | 5 μg/mL ≈ | 10 ng/spot |
| e) | 1 μg/mL ≈ | 2 ng/spot |
| f) | 500 μg/mL ≈ | 1 ng/spot |

After the samples were dabbed on, the membrane was dried for 10 minutes at room temperature before the immune detection protocol was started.

Transfection and Expression of RGM A Fragments in HEK 293F Cells

The protocol developed by Invitrogen for transfection of HEK 293F cells was used for this purpose. The cells were cultured in Free Style 293 expression medium over a period of 2-3 days, and were then centrifuged at 400×g and the supernatant was discarded. The cell pellet was resuspended in medium and adjusted to 3×10⁷ cells in 28 mL fresh medium. The cell pellet was transferred to a 125-mL Erlenmeyer flask and incubated in an incubator at 37° C. and 8% CO₂ on an orbital shaker at 150 rpm until the transfection mixture was produced.

Transfection mixtures with 293fectin DNA complex were prepared as follows:
(1) 30 μg DNA was diluted with Opti-MEM Ito a total volume of 1000 μL and mixed (1000 μL Opti-MEM I was used as control).
(2) 35 μL 293fectin (Invitrogen #12347-019; 1 mL) was diluted with Opti-MEM I to a total volume of 1000 μL, mixed, and incubated for 5 minutes at room temperature.
(3) The DNA mixture from step 1 and the 293fectin solution were transferred to a new test tube and carefully mixed, and after incubation for 25 minutes at room temperature were added to the cells in the Erlenmeyer flask.

The cells were incubated with this transfection mixture for 40 to 48 hours as described above in an incubator at 37° C., 8% CO₂, on an orbital shaker at 150 rpm. The cell supernatants were harvested by centrifuging for 10 minutes at 400×g.

Purification of Proteins using Ni Chelate Affinity (Ni-NTA)

Ni-NTA Superflow beads (Qiagen #1018611) were used. The beads were washed for 3 minutes in phosphate-buffered saline (PBS) solution (Invitrogen) by centrifuging the bead suspension at 13,500 rpm. The supernatants were discarded, and the beads were resuspended in fresh PBS. 200 μL of the bead suspension was used for 30 mL of cell culture supernatant. The beads were incubated with the cell culture supernatants overnight at 4° C. on a shaker (60 rpm), and after incubation were centrifuged (10 minutes, 3000 rpm) in order to pelletize the beads. The supernatants were discarded, and the beads were washed three times with PBS. Bound proteins were eluted from the beads, using 250 μL elution buffer (PBS, 160 mM NaCl, 150 mM imidazole). After incubation for 30 minutes on a shaker at room temperature, the beads were pelletized by centrifugation (3 minutes, 13,500 rpm). The supernatants were collected. The eluted protein was frozen at −20° C. for further analysisimmune detection

For immune detection of immobilized proteins on nitrocellulose, nonspecific binding of proteins was blocked by 1-hour incubation of the blots overnight in TTBS (0.1% Tween 20, tris-buffered saline solution (TBS)) at room temperature or 4° C. The primary antibody was used in a concentration of 10 μg/mL in TTBS for a period of 2 hours at room temperature. The blots were washed three times in TTBS and diluted with secondary antibody (alkaline phosphatase-conjugated anti-mouse IgG-antibody; Sigma) 1:5000 in TTBS, then incubated for 1 hour at room temperature. The blots were washed three times in TTBS and developed with the AP substrate NBT/BCIP (Roche, 1 tablet dissolved in 10 mL purified water) over a period of 3 minutes by covering the blot with the staining solution. The staining reaction was terminated by adding purified water to the blot.

Test Method 1: Illustration of the Effect of RGM Peptides in the Neurite Growth Assay, Using SH-SY5Y Cells

SH-SY5Y cells are human neuroblastoma cells. Blastomas are embryonic tumors which originate during tissue and organ formation. The origin of the blastoma cells is frequently unknown, since in early embryonic states the differentiation of many cells is still premature; i.e., the blastoma cells are a heterogeneous cell population. The cells of a neuroblastoma, referred to as neuroblasts, originate from the neural crest (structure of the embryonic state) from the autonomous nerve tissue, and are practically arrested in an immature stage. The cells in the present case originated from a clonal subculture of the neuroepitheliomal cell line SK-N-SH which were isolated in 1970 in a bone marrow biopsy of a 4-year old girl with metastases of the neuroblastomas.

[http://www.dsmz.de/human_and_animal_cell_lines]

Culture Medium for SH-SY5Y Cells (ECACC No. 94030304)

| | |
|---|---|
| 250 mL | 50% EBSS (Invitrogen) |
| 250 mL | 50% F12 (Ham) NUT MIX + Glutamax I |
| | (Invitrogen) |
| 50 mL | 10% FBS (JRH Biosciences) (heat-inactivated) |
| 5 mL | 1% NEAA (Sigma) (100x) |
| 5 mL | 1% penicillin/streptomycin (Invitrogen) |

The medium was sterile-filtered using a Nunc 500-mL filter, and was stored in a refrigerator at 4° C. until used. Before use, the medium was heated to 37° C. in a water bath.

SH-SY5Y cells are epithelial, neuron-like cells which slowly adherently grow in a monolayer and never attain a confluence of 100% (maximum 80%). The cell culture was split 1:3 two times per week. Isolation of the cells requires incubation with trypsin for 1-3 minutes in an incubator.

Since this is a population of still immature precursor cells, it is possible to differentiate the cells using retinoic acid, so that a certain percentage of the cells acquire neurite-like extensions. For this purpose the culture was incubated in the medium with 10 μM retinoic acid directly in the culture dish or flask, depending on the test, for 3 days.

Microtiter plates (96-well) (containing collagen I-coated plates) were additionally coated with the hRGM A fragments. After washing two times with PBS the cells were seeded. 18-24 hours later the cultures were fixed and stained. In the quantitative analysis the SH-SY5Y cells grown on hRGM A fragments were compared to SH-SY5Y grown on collagen I alone. The neurite length of the cells was automatically measured and used for the analysis.

Test Method 2: Illustration of the Effect of RGM Peptides in the Neurite Growth Test, Using NTera-2 Cells

The human pluripotent cancer cell line NTera2 (DSMZ ACC527) is established as a cell culture model. Neurites grow from cell aggregates and form a corona of neurites around the particular aggregate.

NTera-2 cells are human embryonic teratocarcinoma cells. A carcinoma is a cancerous tumor, whereas a teratoma or teratocarcinoma is a mixed tumor of the germ cells composed of various differentiated and undifferentiated tissues, and therefore, the same as for the SH-SY5Y culture, comprises a heterogeneous population. The tumor is usually present in an encapsulated form in the various types, such as hair, skin, teeth, muscle, and nerve tissue. The tumors typically originate in the ovaries, testes, abdominal cavity, or brain. The cell line was cloned from the Tera-2 line, obtained from a metastatic teratocarcinoma in a 22 year-old Caucasian male.

[http://de.wikipedia.org/wiki/][http://vvww.dsmz.de/human_and_animal_cell_lines]

NTera-2 cells are epithelial, adherently growing cells which form a monolayer. Due to the large number of granular particles which they contain, NTera-2 cells may be easily differentiated from the other cells. The cells were cultivated by 1:5 splitting twice a week. The cells of this mixed culture may be differentiated using retinoic acid in neuronal cells.

Culture Medium Fur Growth

| | |
|---|---|
| 500 mL | Dulbecco's Modified Eagle Medium (DMEM) |
| 50 mL | 10% FBS (heat-inactivated) |
| 10 mL | 5% equine serum (HS) (heat-inactivated) |

The medium was sterile-filtered using a Nunc 500-mL filter, and was stored in a refrigerator at 4° C. until used. Before use, the medium was heated to 37° C. in a water bath.

Differentiation of NTera-2

In order to differentiate the cells it was necessary to introduce antibiotic into the culture medium of the cell line due to the fact that the cells remained in the same culture flask for three weeks. Previously, the undifferentiated culture had been detrypsinated and the cell count determined using a Neubauer counting chamber. 2.5 million cells together with 25 mL medium were transferred to a new culture flask. 25 μL retinoic acid (10 μM) was readded to the medium under dim light conditions. The culture was stored in aliquots (10 M) in a refrigerator, and before use was resuspended at 22° C. in a Thermomixer.

Culture Medium for Differentiation

| | |
|---|---|
| 500 mL | Dulbecco's Modified Eagle Medium (DMEM) |
| 50 mL | 10% FBS (heat-inactivated) |
| 10 mL | 5% equine serum (HS) (heat-inactivated) |
| 5 mL | 1% penicillin/streptomycin |

The culture was stored in an incubator at 37° C. with 5% CO₂ gassing, and the medium was changed at the beginning and at the end of the week. Over time the cells no longer grew in the form of a monolayer, but instead formed small piles of cell aggregations which were visible, without the aid of a light-optical microscope, as light points on the cell layer. For further cultivation, the medium was changed by suctioning off the old medium, washing with 10 mL PBS, then adding 25 mL fresh culture medium to the retinoic acid. After three weeks the cells were differentiated and were ready for use in experiments.

Plate Coating for NTera-2

Growth of the NTera-2 cells on the base of culture dishes was facilitated by coating with poly-L-lysine/laminin.

Different plate formats were used for the tests. For the isolation of protein, 6-well plates were necessary for recovering a sufficient quantity. For the RNA isolation and the immunofluorescence the 24-well-plates were adequate, whereas the assay was established primarily in the 96-well plates. Different volumes of coating and washes were used, corresponding to the capacity of the wells, as shown in the table.

First a poly-L-lysine solution (100 μg/mL) was placed in the wells and incubated for 15 minutes at room temperature. The solution was then suctioned off, and the wells were washed 3× with PBS for 5-10 minutes. A washing step was then performed using sterile Millipore water.

After the washing, a laminin solution (20 μg/mL) was pipetted into the wells, and the plate was incubated for 2 hours at 37° C. with 5% CO₂ gassing in the incubator. Washing with PBS was performed 3× once again for 5-10 minutes, and lastly the PBS was replaced by neurobasal medium, with or without pen[icillin]/strep[tomycin].

| | Poly-L-lysine | |
|---|---|---|
| Type of plate | or laminin | PBS |
| 6-well | 1 mL | 2 mL |
| 24-well | 250 μL | 500 μL |
| 24-well + glass plates | 350 μL | 500 μL |
| 96-well | 50 μL | 100 μL |

Neurite Outgrowth Using NTera-2

After 3 weeks of differentiation, the culture was split 1: 6 over 6 culture flasks in order to select the neuronal cells. At this time the medium was no longer combined with retinoic acid. Within the next two days [and] on the third day the cells were harvested, and the neuronal cells had preferably sedimented on other, non-neuronal cells, but did not adhere too strongly, so that as the top layer they could be easily knocked off. For this purpose the culture medium was removed and washed with approximately 10 mL PBS, and 10 mL PBS was readded to the flask. The cells gradually became dislodged from the base while the bottle was tapped on the side. However, since the non-neuronal cells were supposed to remain adherent, visual checks were occasionally made under a light-optical microscope. The neuronal cells were identified by their brightly illuminated edge and very globular shape. If the neuronal cells have grown on too firmly, they retain the neurites for a short time after being knocked off. The PBS-cell solution in each of 3 flasks was combined in a 50-mL centrifuge tube, and cells were centrifuged for 5 minutes at 1000 rpm at room temperature. The supernatant was then suctioned off into the 2 test tubes, and the pellets were resuspended in 10 mL of neurobasal medium, i.e., combined in 10 mL. The cell count was determined using the Neubauer counting chamber. The neurobasal medium is a specialized medium for further cultivation of the previously differentiated NTera-2 cells.

Culture Medium

| | |
|---|---|
| 100 mL | Neurobasal medium¹⁾ |
| 2 mL | 2% B27 supplement |
| 1 mL | 1% 2 mM L-glutamine |
| 1 mL | 1% penicillin/streptomycin |

¹⁾Neurobasal medium (Gibco/Invitrogen 21103-049)

Formation of Aggregates Using Differentiated NTera-2

For formation of cell aggregates, the knocked-off, differentiated cells (see previous section) which were dissolved in neurobasal medium were diluted with neurobasal medium to a concentration of approximately 1 million cells/mL after the cell count determination. 20 mL of this cell suspension was transferred to a sterile 100-mL disposable shaking flask and incubated overnight, with constant agitation, in an incubator at 37° C. and 5% CO₂ gassing. It was important not to exceed a volume of 20 mL, since otherwise rotation of the liquid does not occur and the cells do not form satisfactory aggregates.

The 96-well plates coated with polylysine/laminin were additionally coated with the hRGM A fragments. After washing 2× with PBS the NTera aggregates were seeded. 18-24 hours later the cultures were fixed and stained. In the quantitative analysis the NTera aggregates grown on hRGM A fragments were compared to NTera aggregates grown on polylysine/laminin alone. The neurite length of the NTera aggregates was automatically measured according to the method described by Lingor et al. (J. Neurochem, 2007, published in electronic form).

The inhibitory effect of RGM peptides and fragments was analyzed by adding different concentrations of the substances to be tested. Alternatively, RGM fragments were provided as substrate.

Test Method 3: RGM A—Neogenin Binding Test

a) Materials:
Immunoplate: Cert. MaxiSorp F96 (Nunc, 439454)
Recombinant human RGM A, R&D Systems; Prod. #2495-RM (260 μg/mL)
Recombinant human neogenin-Fc, Abbott; Ludwigshafen (ALU 1514/122; 425 μg/mL)
Peroxidase-conjugated, affinity-purified mouse anti-human IgG-Fc fragment AK (Jackson Immuno Research, Code: 209-035-098 (0.8 mg/mL))
Developer substrate: ImmunoPure TMB substrate kit (Pierce, #34021)
Sulfuric acid (Merck #4.80354.1000)

b) Method:

1. RGM A binding to immunoplate:
2.5 μg/mL RGM A (R&D) in 50 mM Na₂CO₃ (50 μL/well)
Incubation for 1 hour at 37° C.

2. Washing step:
Wash 3× with PBS/0.02% Tween 20 (100 μL/well)

3. Blocking of nonspecific binding sites
Blocking with 3% BSA in PBS/0.02% Tween (200 μL/well)
Incubation for 1 hour at 37° C.

4. Neogenin binding:
Addition of neogenin in dilutions (initial concentration 1 μg/mL) in 1% BSA PBS/0.02% Tween
Incubation for 1 hour at 37° C.

5. Washing step:
Wash 3× with PBS/0.02% Tween 20 (100 μL/well)

6. Antibody detection of the bound neogenin:
Addition of HRP-coupled mouse anti-human IgG-Fc fragment AK (1:2500 dilution in PBS/1% BSA) (50 μL/well)
Incubation for 1 hour at 37° C.

7. Washing step:
Wash 3× with PBS/0.02% Tween 20 (100 μL/well)

8. Development
Addition of 50 μL developing substrate/well (ImmunoPure TMB substrate, Pierce)
Incubation for 1-30 minutes at room temperature
Stop reaction using 50 μL 2.5 M H₂SO₄/well

Test Method 4: In Vitro Interaction Test for Determining the Interaction between RGMA and BMP-2 and -4

The interaction tests were carried out as described below:

Variant A: Immobilization of the BMP-2/ or -4 Protein and Detection of the Binding of Various RGMA-Fc Fusion Proteins

1. Plate:
Immunoplate Cert. MaxiSorp F96 (Nunc, 439454)

2. Coating:

Recombinant human BMP-2, Catalog No.: 355-BM, Company: R&D Systems; Recombinant human RGM A, R&D Systems; Prod. #2495-RM, or

Recombinant human BMP-4, Catalog No.: 314-BM, Company: R&D Systems;
Concentration: 10 μg/mL
Volume used: 2.5 μg/mL in Na₂CO₃; addition: 50 μL per well
1 hour at 37° C. in damp chamber

3. Washing step:
Wash 3× with PBS/0.025 Tween 20

4. Blocking:
3% BSA in PBS/0.02% Tween, 1 hour at 37° C. in damp chamber; addition: 200 μL per well

5. RGMA peptides:

RGMA-Fc fragments,
1 μg/mL initial concentration, then dilution down to 1:2 using PBS/0.02% Tween20
Incubate for 1 hour at room temperature
1 hour at 37° C. in damp chamber

6. Washing step:
Wash 3× with PBS/0.02% Tween 20

7. Antibody:

Biotin anti-human Fc (R&D-Systems), Catalog No.: 709065; 1 mg/mL;
1:200 in 0.6% BAS/PBS-T (0.02% Tween)
Addition: 50 μL per well
1 hour at 37° C. in damp chamber

8. Secondary antibody: Strep. POD (Roche); Catalog No.: 11089153001
500 U; 1:5000 in 0.6% BAS/PBS-T (0.02% Tween)
Addition: 50 μL per well
1 hour at 37° C. in damp chamber

9. Washing step:
Wash 3× with PBS/0.02% Tween 20

10. Substrate:

ImmunoPure TMB substrate kit; Pierce, #34021
Development period: approximately 1-30 minutes
1:1 mixture of PBS/0.02% Tween 20; addition: 50 μL per well

11. Stop:
2.5 M H₂SO₄; addition: 50 μL per well

Variant B: Immobilization of Various RGMA-Fc Fusion Proteins and Detection of the Binding of the BMP-2/ or -4 Protein

1. Plate:

Immunoplate Cert. MaxiSorp F96 (Nunc, 439454)

2. Coating:

RGMA-Fc fragments
Volume used: 2.5 μg/mL in Na₂CO₃; addition: 50 μL per well
1 hour at 37° C. in damp chamber

3. Washing step:
Wash 3× with PBS/0.02% Tween 20

4. Blocking:
3% BSA in PBS/0.02% Tween, 1 hour at 37° C. in damp chamber
Addition : 200 μL per well, incubation: 1 hour at 37° C. in damp chamber

5. BMP peptides:

Recombinant human BMP-2, Catalog No.: 355-BM, Company: R&D Systems; or

Recombinant human RGM A, R&D Systems; Prod. #2495-RM,

recombinant human BMP-4, Catalog No.: 314-BM, Company: R&D Systems;

Concentration in each case: 10 μg/mL

Dilution steps: in each case 1:2 with PBS/0.02% Tween 20

6. Washing step:
Wash 3× with PBS/0.02% Tween 20

7. Antibody:

Anti-human BMP-4 biotin antibody; Catalog No.: BAM7572, 1:200 in 1% BSA-PBS-T

8. Washing step:
Wash 3× with PBS/0.02% Tween 20

9. Secondary antibody:

Strep. POD (Roche)

Catalog No.: 11089153001; 500 U; 1:5000 in 0.6% BAS/PBS-T (0.02% Tween)
Addition: 50 μL per well
1 hour at 37° C. in damp chamber

10. Washing step:
Wash 3× with PBS/0.02% Tween 20

11. Substrate:

ImmunoPure TMB substrate kit; (Pierce, #34021)
Development period: approximately 1-30 minutes
1:1 mixture of PBS/0.02% Tween 20; addition: 50 μL per well

12. Stop:
2.5 M H₂SO₄; addition: 50 μL per well

Variant C: Inhibition of Binding of Full Length Human RGM A to BMP-4 by MAB 4A9.

1.Plate: Immuno Plate Cert. Maxi Sorp F96 (Fa.NUNC, 439454)

2.Coating:
Recombinant Human BMP-4 Catno.:314-BP
Source: R&D Systems
conc:10 μg/ml
used Volume: 2,5μg/ml in Na₂CO₃/50 μl l per well
1 h incubation at 37° C. in wet chamber

3.Wash: 3× PBS/0.02% Tween20

4.Block: 3%BSA in PBS/0.02% Tween, 1 h incubation at 37° C. in wet chamber, 200 μl per well

5. hRGM A:

#788 RGMA (47-422) 290 μg/ml

ALU 2821/117 11.12.07 Fragment 0.5 μg/ml constant (50 μl)+

MAB4A9 starting concentration: 10 μg/ml,_{—}1:2 dilutons (50 μl)

6.Wash: 3×PBS/0.02% Tween20

7. Primary Detection Antibody:
Biotin anti-human Fc 1 mg/ml
Jackson Immuno Research (Catno.:709-065-149)
1:1000 diluted in 1.5% BSA/PBS-T 75 μl per well
1 h incubation at 37° C. in wet chamber

8.Wash: 3× PBS/0.02% Tween20

9. Secondary Detectiuon Tool:
Streptavidin-coupled Peroxidase (Roche)
Catno.:11089153001, 500U
1:5000 in 1.5% BSA/PBS-T (0.02% Tween)
75 μl per well
1 h incubation at 37° C. in wet chamber

10. Wash: 3× PBS/0.02% Tween20

11. Substrate: Immuno Pure TMB Substrate Kit (Pierce, #34021)Development time: 1-30 min 1:1 mix

12. Stop: 2.5M H₂SO₄

### 2. Production Examples

### Production Example 1

### Production of RGM A Protein Fragments in Mammal Cells

For characterization of the active RGM A, the following RGM A molecules were expressed in mammal cells (HEK293) in the form of Fc fusion proteins:
41-168/Xa
47-90
47-168
316-386
1-450

For this purpose, the DNA which codes for the particular molecule into the vector: pcDNA3.1(+)Zeo IgK/Xa/hIgG lambda he 257-Stop)×HindIII/EcoRI/phosphatase (Invitrogen) was cloned. To this end, the DNA which codes for the particular fragment region was amplified, using PCR, from the RZPD clone (clone AL136826 (DKFZp434D0727); published RZPD sequence: BC015886, AL136826). For this purpose, the oligonucleotide primer listed below, derived from the published RGMA sequence (published sequence: NM_{—}020211), was used.

The PCR was carried out in each case, using these primers and AccuPrime polymerase, on the above-referenced RZPD clone pSport-1 DKFZp434D0727. After purification of the PCR products, digestion with HindIII/EcoRI, and elution of the resulting bands, the desired fragment was ligated in pcDNA3.1(+)Zeo IgK/Xa/hIgG lambda hc 257-Stop)×HindIII/EcoRI/phosphatase. The product obtained was used to transform NEBTurbo cells (Invitrogen) or TOP10 cells (Invitrogen). The resulting clones were checked for correctness of the obtained sequence by means of sequencing.

41-168/Xa:

| (SEQ ID NO: 17) | |
|---|---|
| AM 131: | GGGGAAGCTT [symbol] CCCGCAGCCACCTCC |

(hRGMA sense primer beginning with amino acid); F41 with HindIII segment

AM 132:
(SEQ ID NO: 18)
GGGGGAATTCAAACGACCTTCGAT [symbol] CCCGAAGAGGCCACAGTG

hRGMA antisense primer until amino acid D168 with Factor Xa and EcoRI interface

Transformation into NEBTurbo cells;

Plasmid name: pcDNA3.1(+)Zeo IgK/hRGMA 41-168/Xa/Xa/hIgG lambda hc 257-Stop (no att)

47-90:

| (SEQ ID NO: 19) | |
|---|---|
| AM 169: | GGGGAAGCTTCCGTGCAAGATCCTCAAGTGCAAC |

hRGMA sense primer beginning with amino acid P47 with HindIII segment

| (SEQ ID NO: 20) | |
|---|---|
| AM 171: | CCCCGAATTCAA [symbol] CGTCCGCCGCG |

hRGMA antisense primer until amino acid A90 with EcoRI interface
Transformation into TOP10 cells, Laboratory journal ALU2163/5

Plasmid name: pcDNA3.1(+)Zeo IgK/hRGMA 47-90/Xa/hIgG lambda hc 257-Stop (no att)

47-168:

(SEQ ID NO: 19)

| | |
|---|---|
| AM 169: | GGGGAAGCTT [symbol] TGCAAGATCCTCAAGTGCAAC |

hRGMA sense primer beginning with amino acid P47 with HindIII segment

| (SEQ ID NO: 21) | |
|---|---|
| AM 175: | CCCCGAATTCAA [symbol] CCCGAAGAGGCCACAGTG |

hRGMA antisense primer until amino acid D168 with EcoRI interface

Transformation into TOP10 cells,

Plasmid name: pcDNA3.1(+)Zeo IgK/hRGMA 47-168/Xa/hIgG lambda hc 257-Stop (no att)

316-386:

| | | |
|---|---|---|
| AM 181: | GGGGAAGCTTCTGCGGGGCTGCCCCC | (SEQ ID NO: 22) |

hRGMA sense primer beginning with amino acid L316 with HindIII segment

| (SEQ ID NO: 23) | |
|---|---|
| AM 182: | CCCCGAATTCAAGCCCGTGGTGAGGAGGTCG |

hRGMA antisense primer until amino acid G386 with EcoRI interface

Transformation into TOP10 cells,

Plasmid name: pcDNA3.1(+)Zeo IgK/hRGMA 166-386/Xa/hIgG lambda he 257-Stop (no att)

1-450:

| | | |
|---|---|---|
| Mey 744: | [symbol] CAGCCGCCAAGGGAGAG | (SEQ ID NO: 24) |

hRGMA primer with Start ATG

| | | |
|---|---|---|
| Mey 745: | [symbol] GAACACAGGGAGCAGGGC | (SEQ ID NO: 25) |

hRGMA antisense primer until amino acid C450 without stop

For purposes of comparison, the following Fc fusion proteins were produced in an analogous manner, using additional RGMA; however, their production is not separately described:
266-284
70-120
110-169
169-422
266-335
47-422 Myc His

HEK293F cells (DSMZ No. ACC 305) were transfected with the plasmids, and the expressed fusion protein was isolated as described above.

The analysis of the proteins and the concentration determination were carried out using the methods described above.

### 3. Working Examples

### Working Example 1

### In Vitro Interaction Assay with BMP-4; Comparison of Various RGMA Fragments to RGMA

Fusion proteins tested:

RGMA-Fc

47-168-Fc (“fragment 0”)

218-284-Fc (“fragment 2”)

266-335-Fc (“fragment 3”)

169-422-Fc (“fragment 6”)

The test was conducted according to test method 4, variant B above, with immobilization of the fusion proteins (1 mg/mL). The binding of BMP-4 was detected using anti-BMP-4 biotin antibodies.

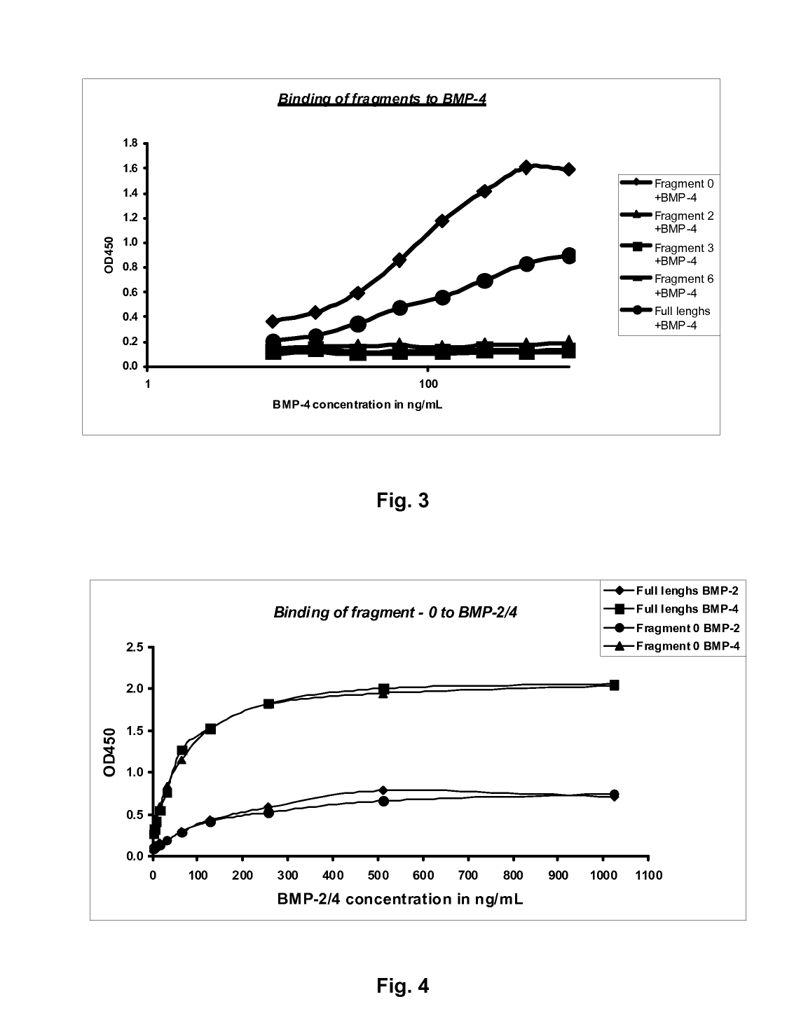

The results are graphically illustrated in FIG. 3. Significant binding of BMP-4 with RGMA in addition to a surprisingly more significant binding with the 47-168 fragment were observed.

### Working Example 2

### In Vitro Interaction Assay with BMP-4 and BMP-2; Comparison of the RGMA Fragment 47-168 to RGMA

Fusion proteins tested:

RGMA-Fc

47-168-Fc (“fragment 0”)

The test was conducted according to test method 4, variant B above, with immobilization of the fusion proteins (1 mg/mL). The binding of BMP-4 and -2 was detected using anti-BMP-4 and anti-BMP-2 biotin antibodies, respectively.

The results are graphically illustrated in FIG. 4. Significant binding of BMP-4 and -2 with RGMA in addition to a surprisingly more significant binding with the 47-168 fragment were observed. BMP-2 and 4 were bound approximately equally strongly in each case.

### Working Example 3

### In Vitro Interaction Assay with BMP-4; Comparison of Various RGMA Fragments

Fusion proteins tested:

47-90-Fc (#785)

47-168-Fc (#786)

316-386-Fc (#790)

169-422-Fc (#769)

70-120-Fc (#779)

110-169-Fc (#780)

266-335-Fc (#789)

47-422Myc-HIS (#801)

The test was conducted according to test method 4, variant A above, with immobilization of BMP-4 (1 mg/mL). The binding of the fusion proteins was detected using anti-human-Fc or anti-Myc anti-rabbit (Invitrogen) antibodies.

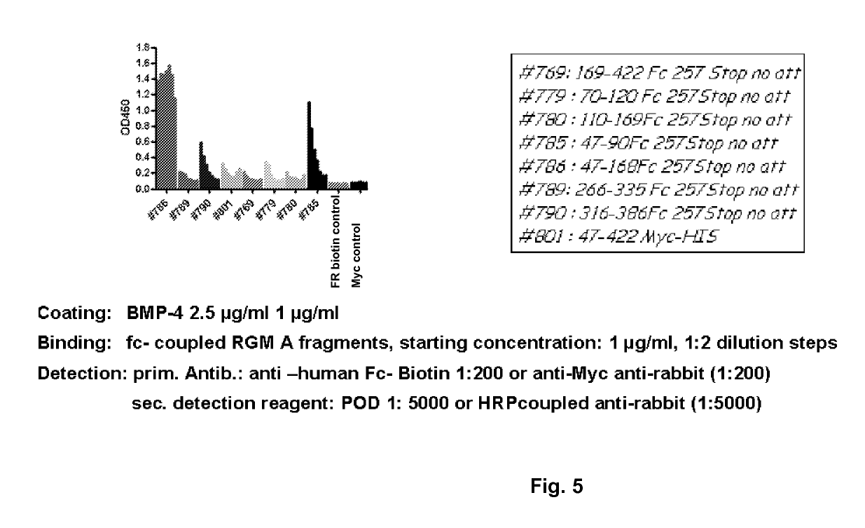

The results are illustrated in accompanying FIG. 5.

Significant concentration-dependent binding of the fragments #785, #786, and #790 according to the invention was observed, with #786 binding the most strongly.

### Working Example 4

### In Vitro Interaction Assay with BMP-4; Comparison of Various Binding RGMA Fragments

Fusion proteins tested:

47-90-Fc (#785)

47-168-Fc (#786)

316-386-Fc (#790)

The test was conducted according to test method 4, variant A above, with immobilization of BMP-4 (1 mg/mL). The binding of the fusion proteins was detected using anti-human-antibodies.

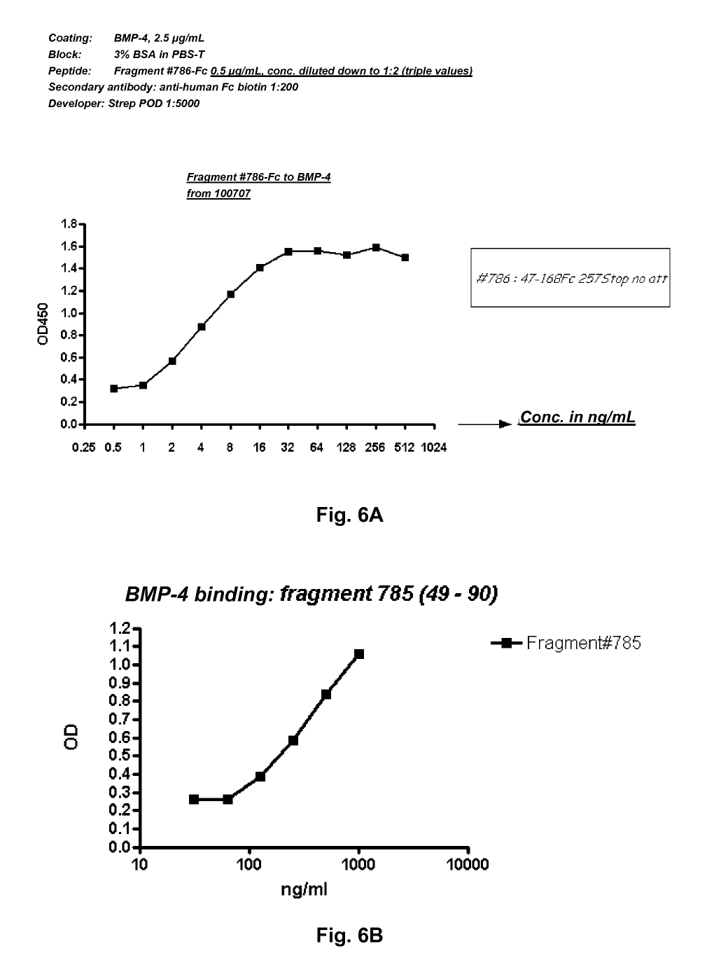

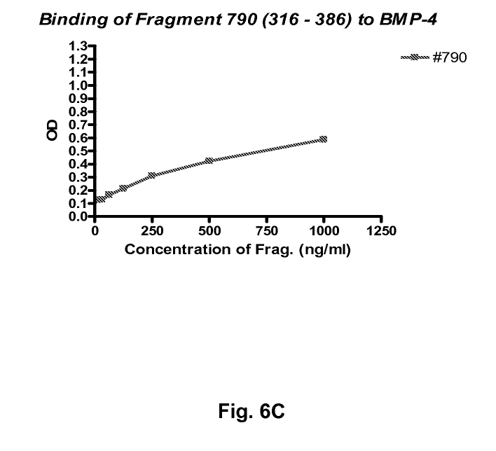

The results are illustrated in FIGS. 6A, B, and C. The results corroborate the findings of exemplary embodiment 3.

### Working Example 5

### Investigation of Synthetic RGM A Fragments in the Nerve Fiber Growth Test

The RGMA fragments listed below

47-168-Fc (#786)

316-386-Fc (#790),

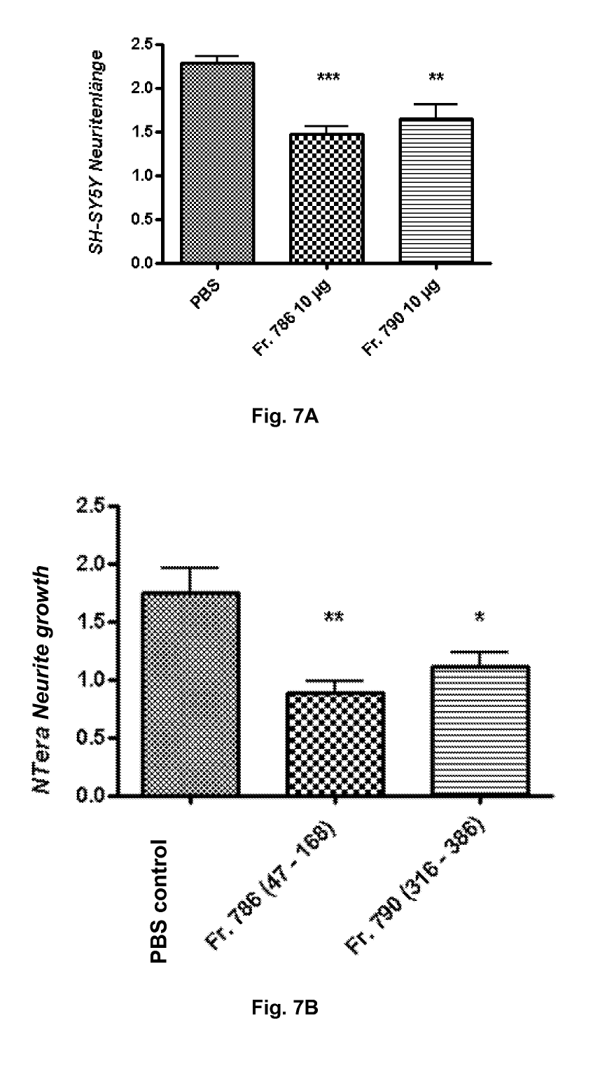

produced according to production example 1, were tested for inhibitory activity in the neurite growth test (see test methods 1 and 2 above) using human NTera nerve cells or human SH-SY5Y cells. The results are illustrated in FIGS. 7A (for SH-SY5Y) and 7B (for NTera).

47-168-Fc (#786) shows significantly higher activity.

Reference is expressly made to the disclosures of the documents cited in the present description section.

### Working Example 6

### Generation and Characterization of Monoclonal Antibody 4A9 Binds to the hRGM A Fragment 47-168.

Unless otherwise stated, standard methods of antibody generation and characterization have been applied.

a) Generation and Immunoblotting

Rats were immunized with full length human RGM A protein.

Sprague-Dawley rats, were immunized and boosted subcutaneously with human RGM A. Animals were injected every three weeks, beginning with a primary injection of 25 μg in complete Freund's adjuvant and injection boosts of 25 μg in Incomplete Freund's Adjuvant. Rats selected for fusion were injected subcutaneously with 25 μg h RGM A in saline, four days prior to fusion. Spleens from immunized animals were removed and single cell suspensions were prepared. SP2/0 myeloma cells were harvested from culture and washed. Spleen cells and tumor cells were mixed at a ratio of 5:1 and fused using 50% PEG 3000 using standard techniques (Kohler and Milstein, 1975). Fused cells were seeded in 96 well plates in selective media, at a density of 2.5×10⁵ spleen cells per well. Fusions were incubated at 37° C. for 7-10 days. When macroscopic colonies were observed, supernatants were removed and tested in the hRGM A ELISA.

Hybridomas that were producing mAbs with desired characteristics were subcloned by the limiting dilution method. Supernatant containing subclones were assayed for binding to hRGM A by ELISA and FACs using HEK 293 cells transfected with hRGM A.

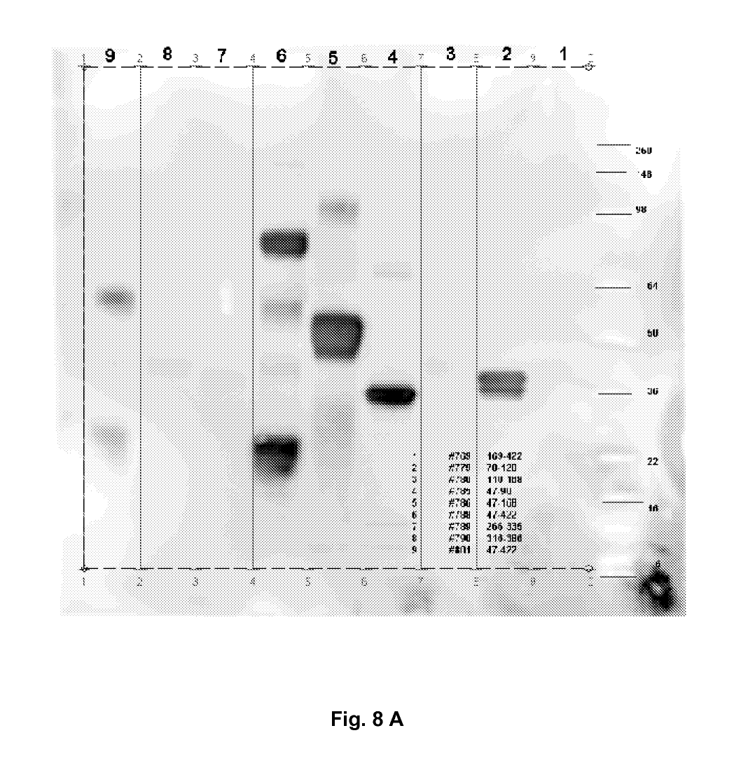

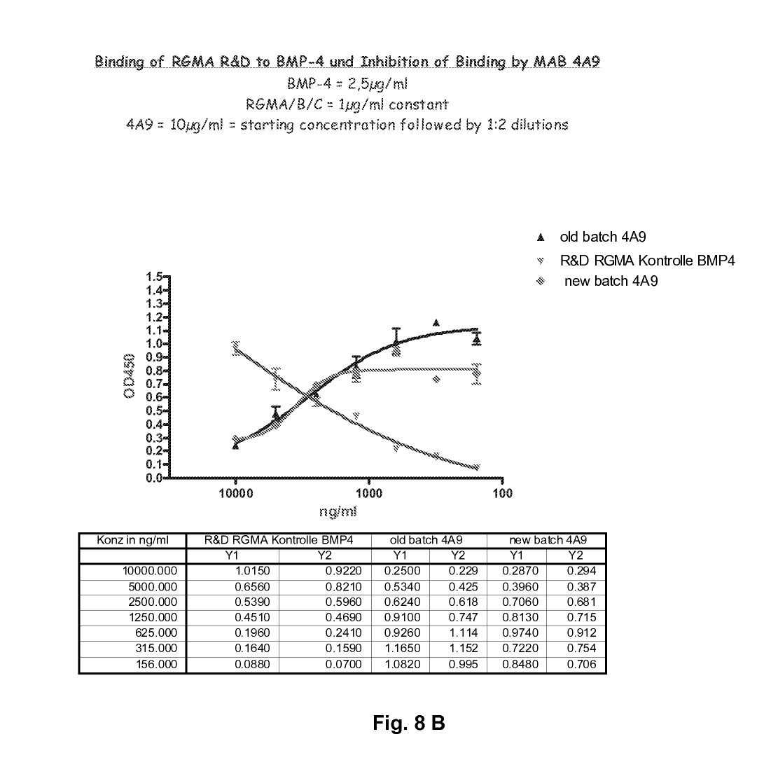

After hybridoma screening using full length human RGM A and fragments of it, MAB 4F9 was isolated because it recognizes fragment 47-168 (lane 5) on western blots (FIG. 8A). This antibody blocked interaction of BMP-4 and full length human RGM A in a solid phase ELISA assay (FIG. 8B).

b) Epitope Mapping of 4A9

Immobilization of the Antibody:

To precisely describe the epitope of MAB 4A9, epitope mapping experiments were performed. To this end 4A9 was bound to a CNBr-activated Seharose resin. Approximately 5-6 nmol of the 4A9 solution (141 μl of 5.76 mg/ml) was added to 20 mg of Sepharose resin, in buffer A (100 mM NaHCO₃, 500 mM NaCl, pH 8) and were mixed for 4 h at room temperature. After washing the antibody conjugated resin 3× with buffer B (100 mM NaHCO₃, 100 mM NaCl, pH 8), hRGM A fragment 47-168 fc (1.18 mg/ml, 1.5 nmol) was added to to the resin with 200 μl buffer B. The mixture was incubated at 4° C. overnight on a rotator. The resin containing antibody 4A9 and antigen hRGM A 47-168 was washed three times with buffer B and was used for epitope excision with Trypsin.

Epitope Excision:

To this aim, the resin containg MAB and antigen were suspended in 200 μl buffer B. 20 μg of trypsin (Promega, Madison Wis.) was dissolved in 200 μl of resuspension buffer (50 mM HOAc), for a concentration of 0.1 μg/μl. Antigen cleavage was performed with 1:100 and 1:200 ratio (w/w) enzyme: antigen, using 0.6 μg and 0.35 μg of trypsihn, respectively. Reaction was done for 7.5 hr, with rotation at 37° C. in a GC oven. After digestio, the resion was washed 2× with buffer B.

Epitope Release:

The remaining antigen peptides that were still bound to the MAB, were released by washing the resin with three 200 μl aliquots of 2% formic acid, and each eluent fraction was collected separately (Elution 1, 2, and 3). The eluted fractions are expected to contain the peptides constituting the MAB 4A9 epitope.

Peptide Analysis by Mass Spectrometry (MS):

After desalting, eluted fractions from the trypsin digestion were subject to mass spectrometric analysis. Matrix-assisted laser desorption (MALDI) MS was performed on a Voyager DE-Pro (Applied Biosystems, Foster City, Calif.) using α-cyano-4-hydroxycinnamic acid (CHCA) matrix (saturated, in 50% acetonitrile , 0.3% TFA). LC-ESI-MS/MS was performed using an Agilent 6510 QTOF MS. Injections of 8 μl were used, and MS/MS was performed on the top 3 ions meeting the specified MS signal criteria. Data were searched using MASCOT (Matrix Science), however most of the data were interpreted manually.

Although all the wash and elution fractions were initially analysed by LC-MS/MS and many by MALDI-MS, data obtained correspond to Elution 1 fractions.

Results:

In the Elution 1 fractions of the trypsin epitope excision experiment the peptides 50-89 and 96-126 of fragment 47-168 of hRGM A were identified. Localisation of the MAB 4A9 peptides in human RGM A is shown below. Fragment 47-168 of hRGM A is marked in bold. The two peptides identified as MAB 4A9 protected peptides are underlined. The precise epitope may be localized within one of these peptides, with the other peptide attached via a disulfide bond.

>hRGMA-NP_064596
MQPPRERLVVTGRAGWMGMGRGAGRSALGFWPTLAFLLCSFPAATSPC
KILKCNSEFWSATSGSHAPASDDTPEFCAALRSYALCTRRTARTCRGD
LAYHSAVHGIEDLMSQHNCSKDGPTSQPRLRTLPPAGDSQERSDSPEI
CHYEKSFHKHSATPNYTHCGLFGDPHLRTFTDRFQTCKVQGAWPLIDN
NYLNVQATNTPVLPGSAATATSKLTIIFKNFQECVDQKVYQAEMDELP
AAFVDGSKNGGDKHGANSLKITEKVSGQHVEIQAKYIGTTIVVRQVGR
YLTFAVRMPEEVVNAVEDWDSQGLYLCLRGCPLNQQIDFQAFHTNAEG
TGARRLAAASPAPTAPETFPYETAVAKCKEKLPVEDLYYQACVFDLLT
TGDVNFTLAAYYALEDVKMLHSNKDKLHLYERTRDLPGRAAAGLPLAP
RPLLGALVPLLALLPVFC

c) Epitope Mapping via Peptide Scanning

Nested, overlapping peptides 15 amino acids in length, spanning the region 51-168 were used to identify the epitope of MAB 4A9. Binding of the peptides was assessed by ELISA and for this, polystyrene plates were coated with the peptides. Peptides were then probed with MAB 4A9 and binding was visualized by ELISA using peroxidase-conjugated anti-rat-fc antibody and TMB substrate.

One reactive peptide was observed for MAB 4A9 and this peptide is shown below.

| | | |
|---|---|---|
| 51 | LKCNSEFWSA TSGSHAPASD DTPEFCAALR SYALCTRRTA | 168 |
| | RTCRGDLAYH SAVHGIEDLM SQHNCSKDGP TSQPRLRTLP | |
| | PAGDSQERSD SPEICHYEKS FHKHSATPNY THCGLFGD | |

MAB 4A9 binds to the peptide highlighted in bold underlined letters and. This peptide is located within amino acids 66-80 and fits were well to one of the peptides identified by epitope excision and mass spectrometric analysis.

### Working Example 7

### Evaluation of the Agonistic or Antagonistic Effect of h RMG A on BMP Signalling using BRE-Luc Assay

a) Materials:
Three compounds were provided at 200 μg/ml.

fragment #785;

fragment #786 and

fragment #788 (IgK/hRGM A 47-422/Xa cleavage site/Fc)
recombinant h BMP-2 (from CHO cells; #355-BM) and monoclonal anti-human BMP2/4 antibodies (#MAB3552) were obtained from R&D Systems Europe, Lille, France
Bright-Glo Luciferase assay kit were purchased from Promega

b) Cell Culture:

BMP-responsive C3H10-B12 (Logeart-Avramoglou D, Bourguignon M, Oudina K, Ten Dijke P, Petite H. An assay for the determination of biologically active bone morphogenetic proteins using cells transfected with an inhibitor of differentiation promoter-luciferase construct. Anal Biochem 2006;349:78-86) were plated in 96-wells plates at a density of 4×10⁴ cells/cm² and cultured in BME (BME=Eagle's Basal Medium) (supplemented with 10% FBS), in a humidified, 37° C., 5% CO₂/95% air environment for 24 h. Cells were rinsed twice with PBS and cultured under BME/0.5% (w/v) BSA containing either each AS compounds tested or anti-human BMP2/4 antibodies (from 0.01 to 10,000 ng/ml) without or with rhBMP-2 (at 50 ng/ml) for 24 h before being assayed for the contained luciferase activity. All experiments were performed in triplicate, and were repeated at two separate times.

c) Results:

In solid phase ELISA experiments, MAB 4A9 completely prevents binding of full length human RGM A to BMP-4, constituting a clear example that the domain of 4A9, residing within the 47-168 human RGM A fragment is important for interaction with BMP-4.

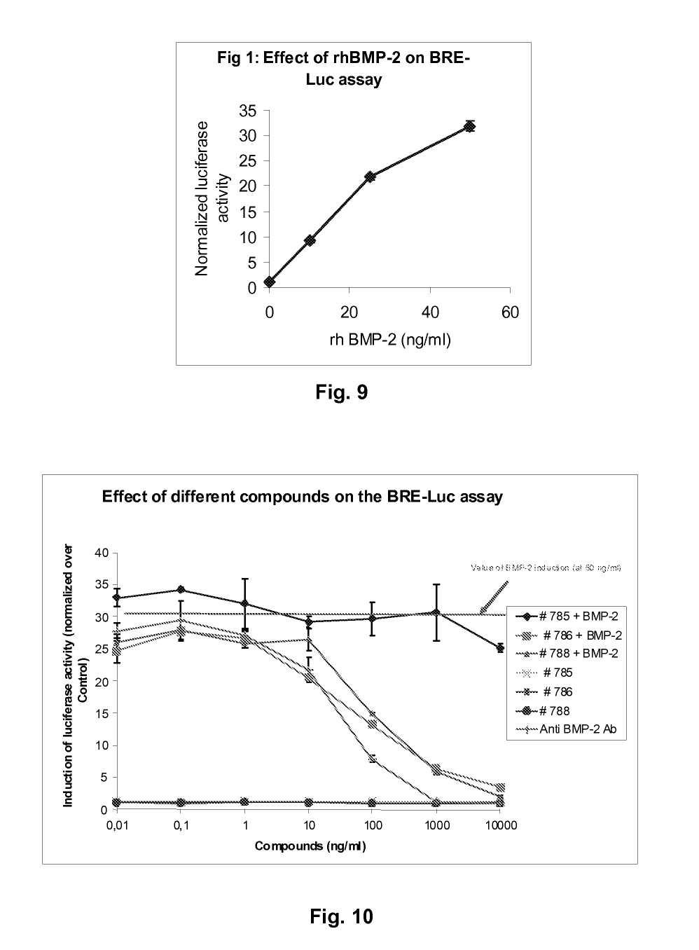

In cellular assays, a dose-dependent response of luciferase activity resulted after treatment of C3H-B12 with rhBMP-2 at different concentrations (from 0 to 50 ng/ml) is shown in FIG. 9.

The agonistic effect of the tested polypetides on BMP signalling using BRE-Luc assay was determined by exposing C3H-B12 to different concentrations of test compounds for 24 hours and monitoring changes in the respective cell luciferase activity (FIG. 10). Any of the three tested compounds induced luciferase activity on their own.

When combined with rhBMP-2 (at 50 ng/ml), fragment #785 did not significantly modify the BMP-2-induced luciferase activity. In contrast, fragments #786 and #788 exhibited a dose-dependent inhibition of the luciferase activity reaching 88% and 93% of inhibition of the rhBMP-2-induced activity when compounds were used at 10 μg/ml. Fragments #786 and #788 demonstrated similar inhibitory effect than anti-rhBMP-2 antibodies used as BMP-2 antagonist control. The equivalent dose for 50% of inhibition (ED50) values for fragments #786 and #788 and anti-BMP-2 Ab are 100 ng/ml, ˜80 ng/ml and ˜50 ng/ml respectively.

d) Conclusion:

The test compounds on their own do not induce BMP signalling pathway, and consequently cannot be considered as BMP agonists. In contrast, when combined with rhBMP-2, two of them (fragments #786 and #788) inhibited the rhBMP-2-induced activity in a dose dependent fashion. Complex formation of such compounds with the BMP-2 protein may impair the binding of BMP-2 to cell membrane receptors and, subsequently, prevented BMP-mediated signalling. However, considering the cell model used for this experiment, a direct intracellular inhibitory effect of the test compounds on the BMP-2 signalling pathway cannot be excluded.

The disclosure of thr documents cited herein is incorporated by reference.

TABLE A
Peptides 10 to 40 aa in length derived from RGM A binding domain 47-168 (Positions according to SEQ ID NO: 2)

| | 1^{st} amino acid residue | | | | | | | | | | | | | | | | | | | | | | |
|---|---|---|---|---|---|---|---|---|---|---|---|---|---|---|---|---|---|---|---|---|---|---|---|
| peptide | 47 | 48 | 49 | 50 | 51 | 52 | 53 | 54 | 55 | 56 | 57 | 58 | 59 | 60 | 61 | 62 | 63 | 64 | 65 | 66 | 67 | 68 | 69 |
| length | last amino acid residue | | | | | | | | | | | | | | | | | | | | | | |
| 10 | 56 | 57 | 58 | 59 | 60 | 61 | 62 | 63 | 64 | 65 | 66 | 67 | 68 | 69 | 70 | 71 | 72 | 73 | 74 | 75 | 76 | 77 | 78 |
| 11 | 57 | 58 | 59 | 60 | 61 | 62 | 63 | 64 | 65 | 66 | 67 | 68 | 69 | 70 | 71 | 72 | 73 | 74 | 75 | 76 | 77 | 78 | 79 |
| 12 | 58 | 59 | 60 | 61 | 62 | 63 | 64 | 65 | 66 | 67 | 68 | 69 | 70 | 71 | 72 | 73 | 74 | 75 | 76 | 77 | 78 | 79 | 80 |
| 13 | 59 | 60 | 61 | 62 | 63 | 64 | 65 | 66 | 67 | 68 | 69 | 70 | 71 | 72 | 73 | 74 | 75 | 76 | 77 | 78 | 79 | 80 | 81 |
| 14 | 60 | 61 | 62 | 63 | 64 | 65 | 66 | 67 | 68 | 69 | 70 | 71 | 72 | 73 | 74 | 75 | 76 | 77 | 78 | 79 | 80 | 81 | 82 |
| 15 | 61 | 62 | 63 | 64 | 65 | 66 | 67 | 68 | 69 | 70 | 71 | 72 | 73 | 74 | 75 | 76 | 77 | 78 | 79 | 80 | 81 | 82 | 83 |
| 16 | 62 | 63 | 64 | 65 | 66 | 67 | 68 | 69 | 70 | 71 | 72 | 73 | 74 | 75 | 76 | 77 | 78 | 79 | 80 | 81 | 82 | 83 | 84 |
| 17 | 63 | 64 | 65 | 66 | 67 | 68 | 69 | 70 | 71 | 72 | 73 | 74 | 75 | 76 | 77 | 78 | 79 | 80 | 81 | 82 | 83 | 84 | 85 |
| 18 | 64 | 65 | 66 | 67 | 68 | 69 | 70 | 71 | 72 | 73 | 74 | 75 | 76 | 77 | 78 | 79 | 80 | 81 | 82 | 83 | 84 | 85 | 86 |
| 19 | 65 | 66 | 67 | 68 | 69 | 70 | 71 | 72 | 73 | 74 | 75 | 76 | 77 | 78 | 79 | 80 | 81 | 82 | 83 | 84 | 85 | 86 | 87 |
| 20 | 66 | 67 | 68 | 69 | 70 | 71 | 72 | 73 | 74 | 75 | 76 | 77 | 78 | 79 | 80 | 81 | 82 | 83 | 84 | 85 | 86 | 87 | 88 |
| 21 | 67 | 68 | 69 | 70 | 71 | 72 | 73 | 74 | 75 | 76 | 77 | 78 | 79 | 80 | 81 | 82 | 83 | 84 | 85 | 86 | 87 | 88 | 89 |
| 22 | 68 | 69 | 70 | 71 | 72 | 73 | 74 | 75 | 76 | 77 | 78 | 79 | 80 | 81 | 82 | 83 | 84 | 85 | 86 | 87 | 88 | 89 | 90 |
| 23 | 69 | 70 | 71 | 72 | 73 | 74 | 75 | 76 | 77 | 78 | 79 | 80 | 81 | 82 | 83 | 84 | 85 | 86 | 87 | 88 | 89 | 90 | 91 |
| 24 | 70 | 71 | 72 | 73 | 74 | 75 | 76 | 77 | 78 | 79 | 80 | 81 | 82 | 83 | 84 | 85 | 86 | 87 | 88 | 89 | 90 | 91 | 92 |
| 25 | 71 | 72 | 73 | 74 | 75 | 76 | 77 | 78 | 79 | 80 | 81 | 82 | 83 | 84 | 85 | 86 | 87 | 88 | 89 | 90 | 91 | 92 | 93 |
| 26 | 72 | 73 | 74 | 75 | 76 | 77 | 78 | 79 | 80 | 81 | 82 | 83 | 84 | 85 | 86 | 87 | 88 | 89 | 90 | 91 | 92 | 93 | 94 |
| 27 | 73 | 74 | 75 | 76 | 77 | 78 | 79 | 80 | 81 | 82 | 83 | 84 | 85 | 86 | 87 | 88 | 89 | 90 | 91 | 92 | 93 | 94 | 95 |
| 28 | 74 | 75 | 76 | 77 | 78 | 79 | 80 | 81 | 82 | 83 | 84 | 85 | 86 | 87 | 88 | 89 | 90 | 91 | 92 | 93 | 94 | 95 | 96 |
| 29 | 75 | 76 | 77 | 78 | 79 | 80 | 81 | 82 | 83 | 84 | 85 | 86 | 87 | 88 | 89 | 90 | 91 | 92 | 93 | 94 | 95 | 96 | 97 |
| 39 | 76 | 77 | 78 | 79 | 80 | 81 | 82 | 83 | 84 | 85 | 86 | 87 | 88 | 89 | 90 | 91 | 92 | 93 | 94 | 95 | 96 | 97 | 98 |
| 31 | 77 | 78 | 79 | 80 | 81 | 82 | 83 | 84 | 85 | 86 | 87 | 88 | 89 | 90 | 91 | 92 | 93 | 94 | 95 | 96 | 97 | 98 | 99 |
| 32 | 78 | 79 | 80 | 81 | 82 | 83 | 84 | 85 | 86 | 87 | 88 | 89 | 90 | 91 | 92 | 93 | 94 | 95 | 96 | 97 | 98 | 99 | 100 |
| 33 | 79 | 80 | 81 | 82 | 83 | 84 | 85 | 86 | 87 | 88 | 89 | 90 | 91 | 92 | 93 | 94 | 95 | 96 | 97 | 98 | 99 | 100 | 101 |
| 34 | 80 | 81 | 82 | 83 | 84 | 85 | 86 | 87 | 88 | 89 | 90 | 91 | 92 | 93 | 94 | 95 | 96 | 97 | 98 | 99 | 100 | 101 | 102 |
| 35 | 81 | 82 | 83 | 84 | 85 | 86 | 87 | 88 | 89 | 90 | 91 | 92 | 93 | 94 | 95 | 96 | 97 | 98 | 99 | 100 | 101 | 102 | 103 |
| 36 | 82 | 83 | 84 | 85 | 86 | 87 | 88 | 89 | 90 | 91 | 92 | 93 | 94 | 95 | 96 | 97 | 98 | 99 | 100 | 101 | 102 | 103 | 104 |
| 37 | 83 | 84 | 85 | 86 | 87 | 88 | 89 | 90 | 91 | 92 | 93 | 94 | 95 | 96 | 97 | 98 | 99 | 100 | 101 | 102 | 103 | 104 | 105 |
| 38 | 84 | 85 | 86 | 87 | 88 | 89 | 90 | 91 | 92 | 93 | 94 | 95 | 96 | 97 | 98 | 99 | 100 | 101 | 102 | 103 | 104 | 105 | 106 |
| 39 | 85 | 86 | 87 | 88 | 89 | 90 | 91 | 92 | 93 | 94 | 95 | 96 | 97 | 98 | 99 | 100 | 101 | 102 | 103 | 104 | 105 | 106 | 107 |
| 40 | 86 | 87 | 88 | 89 | 90 | 91 | 92 | 93 | 94 | 95 | 96 | 97 | 98 | 99 | 100 | 101 | 102 | 103 | 104 | 105 | 106 | 107 | 108 |

| | 1^{st} amino acid residue | | | | | | | | | | | | | | | | | |
|---|---|---|---|---|---|---|---|---|---|---|---|---|---|---|---|---|---|---|
| peptide | 70 | 71 | 72 | 73 | 74 | 75 | 76 | 77 | 78 | 79 | 80 | 81 | 82 | 83 | 84 | 85 | 86 | 87 |
| length | last amino acid residue | | | | | | | | | | | | | | | | | |
| 10 | 79 | 80 | 81 | 82 | 83 | 84 | 85 | 86 | 87 | 88 | 89 | 90 | 91 | 92 | 93 | 94 | 95 | 96 |
| 11 | 80 | 81 | 82 | 83 | 84 | 85 | 86 | 87 | 88 | 89 | 90 | 91 | 92 | 93 | 94 | 95 | 96 | 97 |
| 12 | 81 | 82 | 83 | 84 | 85 | 86 | 87 | 88 | 89 | 90 | 91 | 92 | 93 | 94 | 95 | 96 | 97 | 98 |
| 13 | 82 | 83 | 84 | 85 | 86 | 87 | 88 | 89 | 90 | 91 | 92 | 93 | 94 | 95 | 96 | 97 | 98 | 99 |
| 14 | 83 | 84 | 85 | 86 | 87 | 88 | 89 | 90 | 91 | 92 | 93 | 94 | 95 | 96 | 97 | 98 | 99 | 100 |
| 15 | 84 | 85 | 86 | 87 | 88 | 89 | 90 | 91 | 92 | 93 | 94 | 95 | 96 | 97 | 98 | 99 | 100 | 101 |
| 16 | 85 | 86 | 87 | 88 | 89 | 90 | 91 | 92 | 93 | 94 | 95 | 96 | 97 | 98 | 99 | 100 | 101 | 102 |
| 17 | 86 | 87 | 88 | 89 | 90 | 91 | 92 | 93 | 94 | 95 | 96 | 97 | 98 | 99 | 100 | 101 | 102 | 103 |
| 18 | 87 | 88 | 89 | 90 | 91 | 92 | 93 | 94 | 95 | 96 | 97 | 98 | 99 | 100 | 101 | 102 | 103 | 104 |
| 19 | 88 | 89 | 90 | 91 | 92 | 93 | 94 | 95 | 96 | 97 | 98 | 99 | 100 | 101 | 102 | 103 | 104 | 105 |
| 20 | 89 | 90 | 91 | 92 | 93 | 94 | 95 | 96 | 97 | 98 | 99 | 100 | 101 | 102 | 103 | 104 | 105 | 106 |
| 21 | 90 | 91 | 92 | 93 | 94 | 95 | 96 | 97 | 98 | 99 | 100 | 101 | 102 | 103 | 104 | 105 | 106 | 107 |
| 22 | 91 | 92 | 93 | 94 | 95 | 96 | 97 | 98 | 99 | 100 | 101 | 102 | 103 | 104 | 105 | 106 | 107 | 108 |
| 23 | 92 | 93 | 94 | 95 | 96 | 97 | 98 | 99 | 100 | 101 | 102 | 103 | 104 | 105 | 106 | 107 | 108 | 109 |
| 24 | 93 | 94 | 95 | 96 | 97 | 98 | 99 | 100 | 101 | 102 | 103 | 104 | 105 | 106 | 107 | 108 | 109 | 110 |
| 25 | 94 | 95 | 96 | 97 | 98 | 99 | 100 | 101 | 102 | 103 | 104 | 105 | 106 | 107 | 108 | 109 | 110 | 111 |
| 26 | 95 | 96 | 97 | 98 | 99 | 100 | 101 | 102 | 103 | 104 | 105 | 106 | 107 | 108 | 109 | 110 | 111 | 112 |
| 27 | 96 | 97 | 98 | 99 | 100 | 101 | 102 | 103 | 104 | 105 | 106 | 107 | 108 | 109 | 110 | 111 | 112 | 113 |
| 28 | 97 | 98 | 99 | 100 | 101 | 102 | 103 | 104 | 105 | 106 | 107 | 108 | 109 | 110 | 111 | 112 | 113 | 114 |
| 29 | 98 | 99 | 100 | 101 | 102 | 103 | 104 | 105 | 106 | 107 | 108 | 109 | 110 | 111 | 112 | 113 | 114 | 115 |
| 39 | 99 | 100 | 101 | 102 | 103 | 104 | 105 | 106 | 107 | 108 | 109 | 110 | 111 | 112 | 113 | 114 | 115 | 116 |
| 31 | 100 | 101 | 102 | 103 | 104 | 105 | 106 | 107 | 108 | 109 | 110 | 111 | 112 | 113 | 114 | 115 | 116 | 117 |
| 32 | 101 | 102 | 103 | 104 | 105 | 106 | 107 | 108 | 109 | 110 | 111 | 112 | 113 | 114 | 115 | 116 | 117 | 118 |
| 33 | 102 | 103 | 104 | 105 | 106 | 107 | 108 | 109 | 110 | 111 | 112 | 113 | 114 | 115 | 116 | 117 | 118 | 119 |
| 34 | 103 | 104 | 105 | 106 | 107 | 108 | 109 | 110 | 111 | 112 | 113 | 114 | 115 | 116 | 117 | 118 | 119 | 120 |
| 35 | 104 | 105 | 106 | 107 | 108 | 109 | 110 | 111 | 112 | 113 | 114 | 115 | 116 | 117 | 118 | 119 | 120 | 121 |
| 36 | 105 | 106 | 107 | 108 | 109 | 110 | 111 | 112 | 113 | 114 | 115 | 116 | 117 | 118 | 119 | 120 | 121 | 122 |
| 37 | 106 | 107 | 108 | 109 | 110 | 111 | 112 | 113 | 114 | 115 | 116 | 117 | 118 | 119 | 120 | 121 | 122 | 123 |
| 38 | 107 | 108 | 109 | 110 | 111 | 112 | 113 | 114 | 115 | 116 | 117 | 118 | 119 | 120 | 121 | 122 | 123 | 124 |
| 39 | 108 | 109 | 110 | 111 | 112 | 113 | 114 | 115 | 116 | 117 | 118 | 119 | 120 | 121 | 122 | 123 | 124 | 125 |
| 40 | 109 | 110 | 111 | 112 | 113 | 114 | 115 | 116 | 117 | 118 | 119 | 120 | 121 | 122 | 123 | 124 | 125 | 126 |
| | 1^{st} amino acid residue | | | | | | | | | | | | | | | | | |
| peptide | 88 | 89 | 90 | 91 | 92 | 93 | 94 | 95 | 96 | 97 | 98 | 99 | 100 | 101 | 102 | 103 | 104 | 105 |
| length | last amino acid residue | | | | | | | | | | | | | | | | | |
| 10 | 97 | 98 | 99 | 100 | 101 | 102 | 103 | 104 | 105 | 106 | 107 | 108 | 109 | 110 | 111 | 112 | 113 | 114 |
| 11 | 98 | 99 | 100 | 101 | 102 | 103 | 104 | 105 | 106 | 107 | 108 | 109 | 110 | 111 | 112 | 113 | 114 | 115 |
| 12 | 99 | 100 | 101 | 102 | 103 | 104 | 105 | 106 | 107 | 108 | 109 | 110 | 111 | 112 | 113 | 114 | 115 | 116 |
| 13 | 100 | 101 | 102 | 103 | 104 | 105 | 106 | 107 | 108 | 109 | 110 | 111 | 112 | 113 | 114 | 115 | 116 | 117 |
| 14 | 101 | 102 | 103 | 104 | 105 | 106 | 107 | 108 | 109 | 110 | 111 | 112 | 113 | 114 | 115 | 116 | 117 | 118 |
| 15 | 102 | 103 | 104 | 105 | 106 | 107 | 108 | 109 | 110 | 111 | 112 | 113 | 114 | 115 | 116 | 117 | 118 | 119 |
| 16 | 103 | 104 | 105 | 106 | 107 | 108 | 109 | 110 | 111 | 112 | 113 | 114 | 115 | 116 | 117 | 118 | 119 | 120 |
| 17 | 104 | 105 | 106 | 107 | 108 | 109 | 110 | 111 | 112 | 113 | 114 | 115 | 116 | 117 | 118 | 119 | 120 | 121 |
| 18 | 105 | 106 | 107 | 108 | 109 | 110 | 111 | 112 | 113 | 114 | 115 | 116 | 117 | 118 | 119 | 120 | 121 | 122 |
| 19 | 106 | 107 | 108 | 109 | 110 | 111 | 112 | 113 | 114 | 115 | 116 | 117 | 118 | 119 | 120 | 121 | 122 | 123 |
| 20 | 107 | 108 | 109 | 110 | 111 | 112 | 113 | 114 | 115 | 116 | 117 | 118 | 119 | 120 | 121 | 122 | 123 | 124 |
| 21 | 108 | 109 | 110 | 111 | 112 | 113 | 114 | 115 | 116 | 117 | 118 | 119 | 120 | 121 | 122 | 123 | 124 | 125 |
| 22 | 109 | 110 | 111 | 112 | 113 | 114 | 115 | 116 | 117 | 118 | 119 | 120 | 121 | 122 | 123 | 124 | 125 | 126 |
| 23 | 110 | 111 | 112 | 113 | 114 | 115 | 116 | 117 | 118 | 119 | 120 | 121 | 122 | 123 | 124 | 125 | 126 | 127 |
| 24 | 111 | 112 | 113 | 114 | 115 | 116 | 117 | 118 | 119 | 120 | 121 | 122 | 123 | 124 | 125 | 126 | 127 | 128 |
| 25 | 112 | 113 | 114 | 115 | 116 | 117 | 118 | 119 | 120 | 121 | 122 | 123 | 124 | 125 | 126 | 127 | 128 | 129 |
| 26 | 113 | 114 | 115 | 116 | 117 | 118 | 119 | 120 | 121 | 122 | 123 | 124 | 125 | 126 | 127 | 128 | 129 | 130 |
| 27 | 114 | 115 | 116 | 117 | 118 | 119 | 120 | 121 | 122 | 123 | 124 | 125 | 126 | 127 | 128 | 129 | 130 | 131 |
| 28 | 115 | 116 | 117 | 118 | 119 | 120 | 121 | 122 | 123 | 124 | 125 | 126 | 127 | 128 | 129 | 131 | 131 | 132 |
| 29 | 116 | 117 | 118 | 119 | 120 | 121 | 122 | 123 | 124 | 125 | 126 | 127 | 128 | 129 | 130 | 131 | 132 | 133 |
| 39 | 117 | 118 | 119 | 120 | 121 | 122 | 123 | 124 | 125 | 126 | 127 | 128 | 129 | 130 | 131 | 132 | 133 | 134 |
| 31 | 118 | 119 | 120 | 121 | 122 | 123 | 124 | 125 | 126 | 127 | 128 | 129 | 130 | 131 | 132 | 133 | 134 | 135 |
| 32 | 119 | 120 | 121 | 122 | 123 | 124 | 125 | 126 | 127 | 128 | 129 | 130 | 131 | 132 | 133 | 134 | 135 | 136 |
| 33 | 120 | 121 | 122 | 123 | 124 | 125 | 126 | 127 | 128 | 129 | 130 | 131 | 132 | 133 | 134 | 135 | 136 | 137 |
| 34 | 121 | 122 | 123 | 124 | 125 | 126 | 127 | 128 | 129 | 130 | 131 | 132 | 133 | 134 | 135 | 136 | 137 | 138 |
| 35 | 122 | 123 | 124 | 125 | 126 | 127 | 128 | 129 | 130 | 131 | 132 | 133 | 134 | 135 | 136 | 137 | 138 | 139 |
| 36 | 123 | 124 | 125 | 126 | 127 | 128 | 129 | 130 | 131 | 132 | 133 | 134 | 135 | 136 | 137 | 138 | 139 | 140 |
| 37 | 124 | 125 | 126 | 127 | 128 | 129 | 130 | 131 | 132 | 133 | 134 | 135 | 136 | 137 | 138 | 139 | 140 | 141 |
| 38 | 125 | 126 | 127 | 128 | 129 | 130 | 131 | 132 | 133 | 134 | 135 | 136 | 137 | 138 | 139 | 140 | 141 | 142 |
| 39 | 126 | 127 | 128 | 129 | 130 | 131 | 132 | 133 | 134 | 135 | 136 | 137 | 138 | 139 | 140 | 141 | 142 | 143 |
| 40 | 127 | 128 | 129 | 130 | 131 | 132 | 133 | 134 | 135 | 136 | 137 | 138 | 139 | 140 | 141 | 142 | 143 | 144 |

| | 1^{st} amino acid residue | | | | | | | | | | | | | | | | |
|---|---|---|---|---|---|---|---|---|---|---|---|---|---|---|---|---|---|
| peptide | 106 | 107 | 108 | 109 | 110 | 111 | 112 | 113 | 114 | 115 | 116 | 117 | 118 | 119 | 120 | 121 | 122 |
| length | last amino acid residue | | | | | | | | | | | | | | | | |
| 10 | 115 | 116 | 117 | 118 | 119 | 120 | 121 | 122 | 123 | 124 | 125 | 126 | 127 | 128 | 129 | 130 | 131 |
| 11 | 116 | 117 | 118 | 119 | 120 | 121 | 122 | 123 | 124 | 125 | 126 | 127 | 128 | 129 | 130 | 131 | 132 |
| 12 | 117 | 118 | 119 | 120 | 121 | 122 | 123 | 124 | 125 | 126 | 127 | 128 | 129 | 130 | 131 | 132 | 133 |
| 13 | 118 | 119 | 120 | 121 | 122 | 123 | 124 | 125 | 126 | 127 | 128 | 129 | 130 | 131 | 132 | 133 | 134 |
| 14 | 119 | 120 | 121 | 122 | 123 | 124 | 125 | 126 | 127 | 128 | 129 | 131 | 131 | 132 | 133 | 134 | 135 |
| 15 | 120 | 121 | 122 | 123 | 124 | 125 | 126 | 127 | 128 | 129 | 130 | 131 | 132 | 133 | 134 | 135 | 136 |
| 16 | 121 | 122 | 123 | 124 | 125 | 126 | 127 | 128 | 129 | 130 | 131 | 132 | 133 | 134 | 135 | 136 | 137 |
| 17 | 122 | 123 | 124 | 125 | 126 | 127 | 128 | 129 | 130 | 131 | 132 | 133 | 134 | 135 | 136 | 137 | 138 |
| 18 | 123 | 124 | 125 | 126 | 127 | 128 | 129 | 130 | 131 | 132 | 133 | 134 | 135 | 136 | 137 | 138 | 139 |
| 19 | 124 | 125 | 126 | 127 | 128 | 129 | 130 | 131 | 132 | 133 | 134 | 135 | 136 | 137 | 138 | 139 | 140 |
| 20 | 125 | 126 | 127 | 128 | 129 | 130 | 131 | 132 | 133 | 134 | 135 | 136 | 137 | 138 | 139 | 140 | 141 |
| 21 | 126 | 127 | 128 | 129 | 130 | 131 | 132 | 133 | 134 | 135 | 136 | 137 | 138 | 139 | 140 | 141 | 142 |
| 22 | 127 | 128 | 129 | 130 | 131 | 132 | 133 | 134 | 135 | 136 | 137 | 138 | 139 | 140 | 141 | 142 | 143 |
| 23 | 128 | 129 | 130 | 131 | 132 | 133 | 134 | 135 | 136 | 137 | 138 | 139 | 140 | 141 | 142 | 143 | 144 |
| 24 | 129 | 130 | 131 | 132 | 133 | 134 | 135 | 136 | 137 | 138 | 139 | 140 | 141 | 142 | 143 | 144 | 145 |
| 25 | 130 | 131 | 132 | 133 | 134 | 135 | 136 | 137 | 138 | 139 | 140 | 141 | 142 | 143 | 144 | 145 | 146 |
| 26 | 131 | 132 | 133 | 134 | 135 | 136 | 137 | 138 | 139 | 140 | 141 | 142 | 143 | 144 | 145 | 146 | 147 |
| 27 | 132 | 133 | 134 | 135 | 136 | 137 | 138 | 139 | 140 | 141 | 142 | 143 | 144 | 145 | 146 | 147 | 148 |
| 28 | 133 | 134 | 135 | 136 | 137 | 138 | 139 | 140 | 141 | 142 | 143 | 144 | 145 | 146 | 147 | 148 | 149 |
| 29 | 134 | 135 | 136 | 137 | 138 | 139 | 140 | 141 | 142 | 143 | 144 | 145 | 146 | 147 | 148 | 149 | 150 |
| 39 | 135 | 136 | 137 | 138 | 139 | 140 | 141 | 142 | 143 | 144 | 145 | 146 | 147 | 148 | 149 | 150 | 151 |
| 31 | 136 | 137 | 138 | 139 | 140 | 141 | 142 | 143 | 144 | 145 | 146 | 147 | 148 | 149 | 150 | 151 | 152 |
| 32 | 137 | 138 | 139 | 140 | 141 | 142 | 143 | 144 | 145 | 146 | 147 | 148 | 149 | 150 | 151 | 152 | 153 |
| 33 | 138 | 139 | 140 | 141 | 142 | 143 | 144 | 145 | 146 | 147 | 148 | 149 | 150 | 151 | 152 | 153 | 154 |
| 34 | 139 | 140 | 141 | 142 | 143 | 144 | 145 | 146 | 147 | 148 | 149 | 150 | 151 | 152 | 153 | 154 | 155 |
| 35 | 140 | 141 | 142 | 143 | 144 | 145 | 146 | 147 | 148 | 149 | 150 | 151 | 152 | 153 | 154 | 155 | 156 |
| 36 | 141 | 142 | 143 | 144 | 145 | 146 | 147 | 148 | 149 | 150 | 151 | 152 | 153 | 154 | 155 | 156 | 157 |
| 37 | 142 | 143 | 144 | 145 | 146 | 147 | 148 | 149 | 150 | 151 | 152 | 153 | 154 | 155 | 156 | 157 | 158 |
| 38 | 143 | 144 | 145 | 146 | 147 | 148 | 149 | 150 | 151 | 152 | 153 | 154 | 155 | 156 | 157 | 158 | 159 |
| 39 | 144 | 145 | 146 | 147 | 148 | 149 | 150 | 151 | 152 | 153 | 154 | 155 | 156 | 157 | 158 | 159 | 160 |
| 40 | 145 | 146 | 147 | 148 | 149 | 150 | 151 | 152 | 153 | 154 | 155 | 156 | 157 | 158 | 159 | 160 | 161 |
| | 1^{st} amino acid residue | | | | | | | | | | | | | | | | |
| peptide | 123 | 124 | 125 | 126 | 127 | 128 | 129 | 130 | 131 | 132 | 133 | 134 | 135 | 136 | 137 | 138 | 139 |
| length | last amino acid residue | | | | | | | | | | | | | | | | |
| 10 | 132 | 133 | 134 | 135 | 136 | 137 | 138 | 139 | 140 | 141 | 142 | 143 | 144 | 145 | 146 | 147 | 148 |
| 11 | 133 | 134 | 135 | 136 | 137 | 138 | 139 | 140 | 141 | 142 | 143 | 144 | 145 | 146 | 147 | 148 | 149 |
| 12 | 134 | 135 | 136 | 137 | 138 | 139 | 140 | 141 | 142 | 143 | 144 | 145 | 146 | 147 | 148 | 149 | 150 |
| 13 | 135 | 136 | 137 | 138 | 139 | 140 | 141 | 142 | 143 | 144 | 145 | 146 | 147 | 148 | 149 | 150 | 151 |
| 14 | 136 | 137 | 138 | 139 | 140 | 141 | 142 | 143 | 144 | 145 | 146 | 147 | 148 | 149 | 150 | 151 | 152 |
| 15 | 137 | 138 | 139 | 140 | 141 | 142 | 143 | 144 | 145 | 146 | 147 | 148 | 149 | 150 | 151 | 152 | 153 |
| 16 | 138 | 139 | 140 | 141 | 142 | 143 | 144 | 145 | 146 | 147 | 148 | 149 | 150 | 151 | 152 | 153 | 154 |
| 17 | 139 | 140 | 141 | 142 | 143 | 144 | 145 | 146 | 147 | 148 | 149 | 150 | 151 | 152 | 153 | 154 | 155 |
| 18 | 140 | 141 | 142 | 143 | 144 | 145 | 146 | 147 | 148 | 149 | 150 | 151 | 152 | 153 | 154 | 155 | 156 |
| 19 | 141 | 142 | 143 | 144 | 145 | 146 | 147 | 148 | 149 | 150 | 151 | 152 | 153 | 154 | 155 | 156 | 157 |
| 20 | 142 | 143 | 144 | 145 | 146 | 147 | 148 | 149 | 150 | 151 | 152 | 153 | 154 | 155 | 156 | 157 | 158 |
| 21 | 143 | 144 | 145 | 146 | 147 | 148 | 149 | 150 | 151 | 152 | 153 | 154 | 155 | 156 | 157 | 158 | 159 |
| 22 | 144 | 145 | 146 | 147 | 148 | 149 | 150 | 151 | 152 | 153 | 154 | 155 | 156 | 157 | 158 | 159 | 160 |
| 23 | 145 | 146 | 147 | 148 | 149 | 150 | 151 | 152 | 153 | 154 | 155 | 156 | 157 | 158 | 159 | 160 | 161 |
| 24 | 146 | 147 | 148 | 149 | 150 | 151 | 152 | 153 | 154 | 155 | 156 | 157 | 158 | 159 | 160 | 161 | 162 |
| 25 | 147 | 148 | 149 | 150 | 151 | 152 | 153 | 154 | 155 | 156 | 157 | 158 | 159 | 160 | 161 | 162 | 163 |
| 26 | 148 | 149 | 150 | 151 | 152 | 153 | 154 | 155 | 156 | 157 | 158 | 159 | 160 | 161 | 162 | 163 | 164 |
| 27 | 149 | 150 | 151 | 152 | 153 | 154 | 155 | 156 | 157 | 158 | 159 | 160 | 161 | 162 | 163 | 164 | 165 |
| 28 | 150 | 151 | 152 | 153 | 154 | 155 | 156 | 157 | 158 | 159 | 160 | 161 | 162 | 163 | 164 | 165 | 166 |
| 29 | 151 | 152 | 153 | 154 | 155 | 156 | 157 | 158 | 159 | 160 | 161 | 162 | 163 | 164 | 165 | 166 | 167 |
| 39 | 152 | 153 | 154 | 155 | 156 | 157 | 158 | 159 | 160 | 161 | 162 | 163 | 164 | 165 | 166 | 167 | 168 |
| 31 | 153 | 154 | 155 | 156 | 157 | 158 | 159 | 160 | 161 | 162 | 163 | 164 | 165 | 166 | 167 | 168 | |
| 32 | 154 | 155 | 156 | 157 | 158 | 159 | 160 | 161 | 162 | 163 | 164 | 165 | 166 | 167 | 168 | | |
| 33 | 155 | 156 | 157 | 158 | 159 | 160 | 161 | 162 | 163 | 164 | 165 | 166 | 167 | 168 | | | |
| 34 | 156 | 157 | 158 | 159 | 160 | 161 | 162 | 163 | 164 | 165 | 166 | 167 | 168 | | | | |
| 35 | 157 | 158 | 159 | 160 | 161 | 162 | 163 | 164 | 165 | 166 | 167 | 168 | | | | | |
| 36 | 158 | 159 | 160 | 161 | 162 | 163 | 164 | 165 | 166 | 167 | 168 | | | | | | |
| 37 | 159 | 160 | 161 | 162 | 163 | 164 | 165 | 166 | 167 | 168 | | | | | | | |
| 38 | 160 | 161 | 162 | 163 | 164 | 165 | 166 | 167 | 168 | | | | | | | | |
| 39 | 161 | 162 | 163 | 164 | 165 | 166 | 167 | 168 | | | | | | | | | |
| 40 | 162 | 163 | 164 | 165 | 166 | 167 | 168 | | | | | | | | | | |

| | 1^{st} amino acid residue | | | | | | | | | |
|---|---|---|---|---|---|---|---|---|---|---|
| peptide | 140 | 141 | 142 | 143 | 144 | 145 | 146 | 147 | 148 | 149 |
| length | last amino acid residue | | | | | | | | | |
| 10 | 149 | 150 | 151 | 152 | 153 | 154 | 155 | 156 | 157 | 158 |
| 11 | 150 | 151 | 152 | 153 | 154 | 155 | 156 | 157 | 158 | 159 |
| 12 | 151 | 152 | 153 | 154 | 155 | 156 | 157 | 158 | 159 | 160 |
| 13 | 152 | 153 | 154 | 155 | 156 | 157 | 158 | 159 | 160 | 161 |
| 14 | 153 | 154 | 155 | 156 | 157 | 158 | 159 | 160 | 161 | 162 |
| 15 | 154 | 155 | 156 | 157 | 158 | 159 | 160 | 161 | 162 | 163 |
| 16 | 155 | 156 | 157 | 158 | 159 | 160 | 161 | 162 | 163 | 164 |
| 17 | 156 | 157 | 158 | 159 | 160 | 161 | 162 | 163 | 164 | 165 |
| 18 | 157 | 158 | 159 | 160 | 161 | 162 | 163 | 164 | 165 | 166 |
| 19 | 158 | 159 | 160 | 161 | 162 | 163 | 164 | 165 | 166 | 167 |
| 20 | 159 | 160 | 161 | 162 | 163 | 164 | 165 | 166 | 167 | 168 |
| 21 | 160 | 161 | 162 | 163 | 164 | 165 | 166 | 167 | 168 | |
| 22 | 161 | 162 | 163 | 164 | 165 | 166 | 167 | 168 | | |
| 23 | 162 | 163 | 164 | 165 | 166 | 167 | 168 | | | |
| 24 | 163 | 164 | 165 | 166 | 167 | 168 | | | | |
| 25 | 164 | 165 | 166 | 167 | 168 | | | | | |
| 26 | 165 | 166 | 167 | 168 | | | | | | |
| 27 | 166 | 167 | 168 | | | | | | | |
| 28 | 167 | 168 | | | | | | | | |
| 29 | 168 | | | | | | | | | |

| | 1^{st} amino acid residue | | | | | | | | | |
|---|---|---|---|---|---|---|---|---|---|---|
| peptide | 150 | 151 | 152 | 153 | 154 | 155 | 156 | 157 | 158 | 159 |
| length | last amino acid residue | | | | | | | | | |
| 10 | 159 | 160 | 161 | 162 | 163 | 164 | 165 | 166 | 167 | 168 |
| 11 | 160 | 161 | 162 | 163 | 164 | 165 | 166 | 167 | 168 | |
| 12 | 161 | 162 | 163 | 164 | 165 | 166 | 167 | 168 | | |
| 13 | 162 | 163 | 164 | 165 | 166 | 167 | 168 | | | |
| 14 | 163 | 164 | 165 | 166 | 167 | 168 | | | | |
| 15 | 164 | 165 | 166 | 167 | 168 | | | | | |
| 16 | 165 | 166 | 167 | 168 | | | | | | |
| 17 | 166 | 167 | 168 | | | | | | | |
| 18 | 167 | 168 | | | | | | | | |
| 19 | 168 | | | | | | | | | |
| 20 | | | | | | | | | | |
| 21 | | | | | | | | | | |
| 22 | | | | | | | | | | |
| 23 | | | | | | | | | | |
| 24 | | | | | | | | | | |
| 25 | | | | | | | | | | |
| 26 | | | | | | | | | | |
| 27 | | | | | | | | | | |
| 28 | | | | | | | | | | |
| 29 | | | | | | | | | | |

1. A bone morphogenetic protein (BMP)-binding domain of the repulsive guidance molecule (RGM) or a polypeptide fragment thereof or a fusion protein thereof.

2. The BMP-binding domain according to claim 1, derived from RGM of mammals.

3. The BMP-binding domain according to claim 1, derived from a human RGM A according to SEQ ID NO: 2, human RGM B according to SEQ ID NO: 4, or human RGM C according to SEQ ID NO: 6.

4. The BMP-binding domain according to claim 1, localized in an amino acid sequence range having a length of up to approximately 170 and N-terminal with respect to von Willebrand factor domains of RGM A.

5. The BMP-binding domain according to claim 4, having a length of approximately 30 to 150 amino acid radicals, functional derivatives thereof, and fusion proteins, containing at least one BMP-binding domain in functional linkage with at least one additional, different amino acid sequence.

6. The BMP-binding domain according to claim 1, characterized by at least one of the following partial sequences according to SEQ ID NO: 7 and 8:
(SEQ ID NO: 7)
X₁C(K/R)IX₂(K/R)CX₃(S/T/A)(E/D)(F/Y)X₄SX₅T
where X₁ through X₅ stand for any given amino acid radicals; or

| | |
|---|---|
| X₆CX₇ALRX₈YAX₉CTX₁₀RTX₁₁ | (SEQ ID NO: 8) |

where X₆ through X₁₁ stand for any given amino acid radicals;
or a partial sequence of formula
(SEQ ID NO:7)-Link1-(SEQ ID NO:8)
where Link1 stands for a SEQ ID NO: 7- and 8-bridging amino acid sequence containing 13 to 28 any given contiguous amino acid radicals.

7. The BMP-binding domain according to claim 1, containing one of the following amino acid sequences of SEQ ID NO: 2:
Amino acid position of approximately 47 to approximately 168
Amino acid position of approximately 47 to approximately 90 or
Amino acid position of approximately 75 to approximately 121;
or one of the following amino acid sequences of SEQ ID NO: 4:
Amino acid position of approximately 94 to approximately 209
Amino acid position of approximately 94 to approximately 137 or
Amino acid position of approximately 122 to approximately 168;
one of the following amino acid sequences of SEQ ID NO: 6:
Amino acid position of approximately 36 to approximately 172
Amino acid position of approximately 36 to approximately 94 or
Amino acid position of approximately 80 to approximately 125;
or a polypeptide fragment thereof.

8. The BMP-binding domain or a polypeptide fragment thereof according to claim 1, containing at least 10 contiguous amino acid radicals from the sequence range from approximately position 316 to approximately 386 according to SEQ ID NO: 2, from the sequence range from approximately position 350 to approximately 421 according to SEQ ID NO: 4, or from the sequence range from approximately position 314 to 369 according to SEQ ID NO: 6.

9. The BMP-binding domain or a polypeptide fragment according to claim 7, containing at least 10 contiguous amino acid radicals from the sequence range from approximately position 47 to approximately 168 according to SEQ ID NO: 2, from the sequence range from approximately position 94 to approximately 209 according to SEQ ID NO: 4, or from the sequence range from approximately position 36 to 172 according to SEQ ID NO: 6.

10. The BMP-binding domain according to claim 1, which binds to at least one BMP selected from BMP-2, BMP-4, BMP-5, BMP-6, and BMP-12, and in particular binds to BMP-2 and/or BMP-4.

11. The BMP-binding domain according to claim 10, which also binds to neogenin.

12. (canceled)

13. The polypeptide fragment according to claim 1, which may be used for production of immunoglobulin molecules, which modulate the binding of RGM to BMP and/or neogenin.

14. The polypeptide fragment according to claim 12, containing at least 10 contiguous amino acid radicals of a peptide having one of the sequences according to SEQ ID NO: 2, 4, or 6.

15. The fusion protein of claim 1, operatively linked to a second polypeptide selected from a mono- or polyvalent carrier polypeptide or a second biologically active polypeptide.

16. The fusion protein according to claim 15, wherein the polyvalent carrier contains at least one Fc or Fc″, wherein each of the two polypeptide chains thereof is operatively linked to the same or different BMP-binding domain.

17. An antibody against RGM produced using the BMP-binding domain or polypeptide fragment or fusion protein according to claim 1.

18-30. (canceled)

31. A pharmaceutical composition comprising a pharmaceutically acceptable carrier.
and the BMP-binding domain, or a polypeptide fragment thereof, or a fusion protein thereof according to claim 1.

32. The pharmaceutical composition according to claim 31 for intrathecal, intravenous, subcutaneous, oral or parenteral, percutaneous, subdermal, intraosseal, nasal, extracorporeal or inhalation administration.

33-40. (canceled)

41. The pharmaceutical composition according to claim 31, further comprising an antibody against RGM.

42. A method of treating a bone growth disorder, a bone injury, an autoimmune disease selected from the group consisting of: spondylitis ankylosans, antiphospholipid syndrome, Addison's disease, autoimmune hemolytic anemia, autoimmune hepatitis, autoimmune lymphoproliferative syndrome (ALPS), autoimmune thrombocytopenic purpura (ATP), Behcet's disease, bullous and pemphigoid, cardiomyopathy, celiac disease, dermatitis herpetiformis, chronic fatigue immune dysfunction syndrome (CFIDS), chronic inflammatory demyelinating polyneuropathy (CIDP), cicatricial pemphigoid, systemic sclerosis (CREST syndrome), cold agglutination disease, Crohn's disease, cutaneous vasculitis, Degos disease, dermatomyositis, juvenile dermatomyositis, lupus erythematosus discoides, essential mixed cryoglobulinemia, fibromyalgia, Goodpasture's syndrome, Graves' disease, Guillain-Barré syndrome, Hashimoto's thyroiditis, idiopathic pulmonary fibrosis, idiopathic thrombocytopenia purpura (ITP), immunoglobulin A nephropathy, insulin-dependent diabetes mellitus, juvenile arthritis, Kawasaki disease, lichen planus, membranous glomerulonephritis, Meniere's disease, mixed connective tissue disease, multifocal motor neuropathy, multiple sclerosis, myasthenia gravis, pemphigus vulgaris, pernicious anemia, polyarteritis nodosa, polychondritis, polyglandular syndrome, polymyalgia rheumatica, polymyositis, dermatomyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjogren's syndrome, stiff man syndrome, systemic lupus erythematosus, Takayasu's arteritis, temporal arteritis/giant cell arteritis, ulcerative colitis, uveitis, vasculitis, vitiligo, Wegener's granulomatosis or hair loss diseases selected from the group consisting of: alopecia areata, alopecia totalis, alopecia universalis, androgenic alopecia, telogen effluvium, anagen effluvium, and chemotherapy-induced alopecia, the method comprising the step of:
administering to a subject with the pharmaceutical agent of claim 31.
